SEQUENCE LISTING (8 sequences)

SEQ ID NO: 1 - DNA, 69 bp, Homo sapiens
gccaccatggccttaccagtgaccgccttgctcctgccgctggccttgctgctccacgccgccaggccg

SEQ ID NO: 2 - DNA, 321 bp, Homo sapiens
tccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagc

SEQ ID NO: 3 - DNA, 135 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 4 - DNA, 75 bp, Homo sapiens
cctatcttactaaccatcagcattttgagttttttctctgtcgctctgttggtcatcttggcctgtgtgttatgg

SEQ ID NO: 5 - DNA, 585 bp, Homo sapiens
aaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccag

SEQ ID NO: 6 - DNA, 591 bp, Homo sapiens
ggctccggagccacgaacttctctctgttaaagcaagcaggagacgtggaagaaaaccccggtcccatgggtcttttctgcagtcaccgtcctcgaggcaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 7 - DNA, 747 bp, Homo sapiens
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggcaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggtggtggtggttctggtggtggtggttctggcggcggcggctccggtggtggtggatcccaggtccaattgcagcagcctgggtctgagcttgtgaagcctggggcttcagtcaagctgtcctgcaaggcttctggctacaccttcaccagctacttgatgcactgggtgaggcagaggcctggacgaggccttgagtggattggaaggattgatcctaatagtggtggtactaaatacagtgagaagttgaagagcaaggccacactgactgtagacaaaccctccagcacagcctacatgcagctcaggagcctgacatctgaggactctgcggtctattattgtgcaacatattactacggtagtagctactactttgactactggggccaaggcaccactctcacagtctcctca

SEQ ID NO: 8 - DNA, 1140 bp, Homo sapiens
cccaaacccttcatcaccagcaacaattctaaccccgtggaggatgaggatgctgtagccttaacctgtgaacctgagattcagaacacaacctacctgtggtgggtaaataatcagagcctcccggtgagtccaagactgcagctgtctaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccaacagcatacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaataccacttatctctggtgggtgaacggtcagagcctcccagtcagtcccaggctgcagctgtccaatggcaatagaacactgaccctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgata

(19) United States
(12) Patent Application Publication
(10) Pub. No.: US 2020/0147137 A1
(43) Pub. Date: May 14, 2020

(54) COMPOSITIONS AND METHODS FOR TREATMENT OF CANCER

(71) Applicant: WUGEN, Inc., St. Louis, MO (US)

(72) Inventors: John MCKEARN, St. Louis, MO (US); Ayman KABAKIBI, San Diego, CA (US); You W. ZHOU, St. Louis, MO (US)

(21) Appl. No.: 16/679,127

(22) Filed: Nov. 8, 2019

Related U.S. Application Data
(60) Provisional application No. 62/757,628, filed on Nov. 8, 2018.

(51) Int. Cl. A61K 35/17; C07K 14/725; C07K 14/715; C12N 15/85

(52) U.S. Cl. CPC A61K 35/17; C07K 14/7051; C07K 14/7155; C12N 15/85; C07K 2317/54; C07K 2317/622; C07K 2317/515; C07K 2317/51; C12N 2015/8518

(57) ABSTRACT

Disclosed herein are transmembrane proteins comprising at least two chains, each chain comprising an endodomain, a transmembrane domain, and an ectodomain; wherein the ectodomains of the at least two chains of the transmembrane protein interact together to bind to a drug or soluble protein; wherein the endodomains of the at least two chains of the transmembrane protein together comprise a structure functionally similar to that of an IL-2 receptor endodomain, an IL-7 receptor endodomain, or an IL-15 receptor endodomain; and wherein binding of the drug or soluble protein to the ectodomain activates IL-2, IL-7, or IL-15 signaling in CAR-bearing immune effector cells.

This application claims the benefit of priority of U.S. Provisional Application No. 62/757,628, filed on Nov. 8, 2018, the disclosure of which is hereby incorporated by reference as if written herein in its entirety.

Cancer is a common disease worldwide, resulting in thousands of deaths per year. Progression of some tumors has been shown to involve suppression of the normal immunological surveillance function of the immune system of the organism to avoid detection. Cancer patients often have suppressed immune function with reduced T cell counts.

While chemotherapy and radiotherapy remain the most common and effective treatments for cancer, immunotherapy has received increasing interest in recent years. Immunotherapy utilizes the immune system of the patient to kill cancer cells. In particular, the field of adoptive cell therapy with immune effector cells, such as T-cells, bearing cancer-targeting chimeric antigen receptors—CAR-T cells—has experienced enormous development. Several CAR-T cell therapies have been approved for use and several more are under development. Amongst the challenges facing the field of CAR-T therapy are the generation of CAR-T cells in a sufficiently short period of time to treat a critically ill patient, and the expansion and persistence of a population of CAR-T cells in the patient to effect therapy.

Interleukin-7 (IL-7), interleukin-2 (IL-2), and interleukin-15 (IL-15) are cytokines involved in T cell development, maturation, homeostasis, and proliferation. They are also involved in a number of cellular functions and pathways, in which they function to activate numerous intracellular signaling pathways. For example, IL-7 promotes the survival and proliferation of both naïve and memory T cells. IL-7, IL-2, or IL-15, or an analogue thereof, could be administered in vivo to stimulate expansion of CAR-T cells or other CAR-bearing immune effector cells in a patient receiving adoptive cell transfer therapy.

However, IL-7 signaling is known to occur in the initiation and maintenance of some lymphocyte-derived tumors, such as T-cell acute lymphoblastic leukemia, and cutaneous T-cell lymphoma cells secrete more IL-7, which contributes to their proliferation. Elevated IL-7 and constitutive production of IL-7 has been documented in solid tumors. Accordingly, activation of the IL-7, IL-2, or I-15 receptor by administration of IL-7, Il-2, or Il-15 or analogues thereof to stimulate production of CAR-T cells, risks on-target, off-tissue effects that could exacerbate cancer or have other unpredicted effects.

The present disclosure utilizes native IL-7R, IL-2R, and IL-15R signaling pathways, along with modified activity of the IL-7, IL-2, or IL-15 receptor as an immunotherapy for cancer treatment.

### BRIEF DESCRIPTION OF THE DRAWINGS

FIG. 1—Shows, from left to right, a schematic of the structures of native IL-2 receptor (IL-2R), IL-7 receptor (IL-7R), and IL-15 receptor (IL-15R) when each is bound to its IL-2, IL-7 or IL-15 ligand, respectively (see, e.g., Nature Reviews Immunology 5:688-698, 2005). IL-7R comprises two chains, namely The three receptors depicted are not necessarily present, and do not necessarily serve the same function, in the same natural immune effector cell.

FIG. 2—Shows a schematic of a recombinant IL-7 receptor. Administration of a soluble protein comprising two distinct epitopes (domain A and domain B) bind scFv A, linked to the internal signaling domains of the IL-7Rα chain, and scFv B, linked to the internal signaling domains of the common γ chain. Simultaneous binding of the protein to scFv A and scFv B results in dimerization of the recombinant IL-7Rα and the common γ chain and induces IL-7 signaling.

FIG. 3—Shows a schematic of a recombinant IL-7 receptor. The recombinant IL-7Rα chain comprises a transmembrane domain (e.g., the transmembrane domain of il-7ra or CD28), linked to a FKBP binding domain, which in turn is coupled to the intracellular portion of the IL-7Rα chain. The recombinant common gamma chain comprises a transmembrane domain (e.g., the common gamma chain or CD28), linked to the FKBP binding domain, which in turn is coupled to the intracellular portion of the common gamma chain. Administration of a chemically induced dimerization agent, e.g., FK1012, will lead to dimerization of the IL-7Rα chain and the common γ chain and induce IL-7 signaling.

FIG. 4—Shows a schematic of polycistronic DNA constructs designed to express a CAR, a recombinant IL-7Rα chain, and a recombinant common γ chain as three independent proteins. (a) The intracellular domain of an IL-7Rα chain is fused to an scFv via a linker protein (hinge) and a transmembrane domain (TMD). An intracellular γc domain is fused to an scFv via a linker protein (hinge) and a transmembrane domain (TMD). A leader sequence is included for trafficking of individual chains to the cell membrane. 2A peptides separate the CAR, recombinant IL-7Rα chain, and recombinant γc produce independent proteins. (b) The intracellular domain of the IL-7Rα chain is fused to an FKBP domain and a transmembrane domain (TMD). The intracellular γc domain is fused to an FKBP domain and a transmembrane domain (TMD). A leader sequence is included for trafficking of individual chains to the cell membrane. 2A peptides separate the CAR, recombinant IL-7Rα chain, and recombinant γc to produce independent proteins.

FIG. 5—Shows a schematic for inducing IL-2, IL-7, and IL-15 signaling within the same cell. The drug or soluble protein has the capacity to cross-link either the recombinant IL-7Rα to the recombinant γc or the recombinant IL-15Rβ or IL-2Rβ to the recombinant γc. All three recombinant chains are expressed in the same cell. Thus, 50% of dimerization events will induce IL-7R signaling and 50% will induce IL-15R signaling.

FIG. 6—Shows expression of WU46 construct on HEK-293T cells.

FIG. 7—Shows pSTAT5 signaling of unstimulated (left peak) and IL-7-stimulated human primary T cells (right peak).

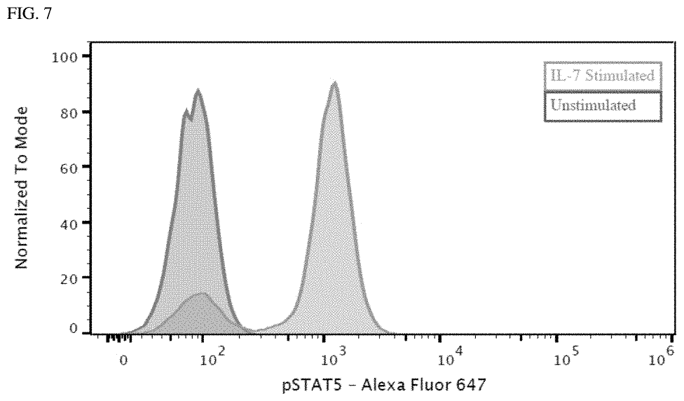

FIG. 8—Shows 293T cells transfected with WU42-44 plasmids. All plasmids used contain P2A_Thy1.1 reporter. Left to right: Negative control, WU42-CD33, WU43-CLEC12A, WU44-IgE_IL7R.

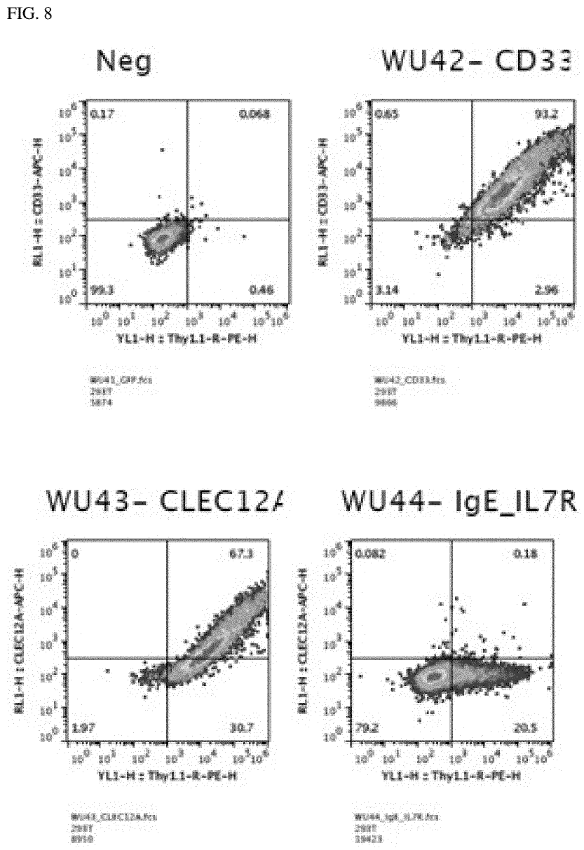

### BRIEF DESCRIPTION OF THE SEQUENCES

SEQ ID NO:1—Sequence of Domain 1 (Kozak and CD8a signal peptide) of Construct 1 (Human IgE CE3 IL-7Ra chimera “WU44”).

SEQ ID NO:2—Sequence of Domain 2 (Human IgE CE3 ECD) of Construct 1 (Human IgE CE3 IL-7Ra chimera “WU44”).

SEQ ID NO:3—Sequence of Domain 3 (CD8 Linker ECD) of Construct 1 (Human IgE CE3 IL-7Ra chimera “WU44”).

SEQ ID NO:4—Sequence of Domain 4 (IL-7Ra TMD) of Construct 1 (Human IgE CE3 IL-7Ra chimera “WU44”).

SEQ ID NO:5—Sequence of Domain 5 (IL-7Ra ICD) of Construct 1 (Human IgE CE3 IL-7Ra chimera “WU44”).

SEQ ID NO:6—Sequence of Domain 6 (P2A-Thy1.1 co-expressed surface marker) of Construct 1 (Human IgE CE3 IL-7Ra chimera “WU44”).

SEQ ID NO:7—Sequence of Domain 2 (Anti-NP 3B44 SCFV ECD) of Construct 2 (Anti-NP clone 3B44 SCFV IL-7Ra chimera “WU45”).

SEQ ID NO:8—Sequence of Domain 2 (CEA a2-b3 ECD) of Construct 3 (Human CEA IL-7Ra chimera “WU46”).

### DETAILED DESCRIPTION OF THE DISCLOSURE

Disclosed herein is Embodiment 1: a composition comprising a population of CAR-bearing immune effector cells, the cells comprising a transmembrane protein, the transmembrane protein comprising at least two recombinant chains, each recombinant chain comprising an endodomain, a transmembrane domain, and an ectodomain;

wherein the ectodomains of the at least two chains of the transmembrane protein interact together to bind to a drug or soluble protein;

wherein the endodomains of the at least two chains of the transmembrane protein together comprise a structure functionally similar to that of an IL-2 receptor endodomain, an IL-7 receptor endodomain, or an IL-15 receptor endodomain; and

wherein binding of the drug or soluble protein to the ectodomain activates IL-2, IL-7, or IL-15 signaling in CAR-bearing immune effector cells.

Also disclosed are the following embodiments.

### Embodiment 2

The composition of embodiment 1, wherein the ectodomains of the at least two chains of the transmembrane protein each comprise an antibody or binding portion thereof.

### Embodiment 3

The composition of embodiment 2, wherein the binding portion of each antibody is chosen from a Fab, scFv, F(ab′)₂, minibody, or other binding arrangement of V_{H} and V_{L} chains or CDR-containing proteins.

### Embodiment 4

The composition of embodiment 2, wherein the ectodomains of the at least two chains of the transmembrane protein each comprise a scFv.

### Embodiment 5

The composition of any of embodiments 2-4, wherein the antibodies or binding portions thereof, or the scFvs, together comprise a drug- or soluble protein-recognition domain.

### Embodiment 6

The composition of any of embodiments 1-5, wherein the soluble protein comprises two distinct epitopes.

### Embodiment 7

The composition of embodiment 6, wherein the two distinct epitopes of the soluble protein bind to the drug- or soluble protein-recognition domain.

### Embodiment 8

The composition of embodiment 7, wherein the binding of the transmembrane protein to the drug- or soluble protein-recognition domain initiates internal signaling.

### Embodiment 9

The composition of any of embodiments 1-8, wherein the at least two chains of the transmembrane protein comprise:

a recombinant IL-7Rα chain or fragment thereof and a recombinant common γ chain or fragment thereof;

two recombinant IL-7Rα chains or fragments thereof;

a recombinant IL-2Rβ chain or fragment thereof and a recombinant common γ chain or fragment thereof; or

two recombinant IL-2Rβ chains or fragments thereof;

a recombinant IL-15Rβ chain or fragment thereof and a recombinant common γ chain or fragment thereof; or

two recombinant IL-15Rβ chains or fragments thereof.

### Embodiment 10

The composition of embodiment 9, wherein the recombinant IL-7Rα chain or fragment thereof, IL-2Rβ chain or fragment thereof, or IL-15Rβ chain or fragment thereof comprises (i) a transmembrane domain, and (ii) an endodomain comprising an IL-7Rα, IL-2Rβ, or IL-15Rβ signaling domain.

### Embodiment 11

The composition of any of embodiments 1-10, wherein the endodomains of the at least two chains of the transmembrane protein comprise:

a recombinant chain or fragment thereof comprising an IL-7Rα signaling domain and a recombinant chain or fragment thereof comprising a common γ signaling domain;

two recombinant chains or fragments thereof each comprising an IL-7Rα signaling domain;

a recombinant chain or fragment thereof comprising an IL-2Rβ signaling domain and a recombinant chain or fragment thereof comprising a common γ signaling domain;

two recombinant chains or fragments thereof each comprising an IL-2Rβ signaling domain;

a recombinant chain or fragment thereof comprising an IL-15Rβ signaling domain and a recombinant chain or fragment thereof comprising a common γ signaling domain; or

two recombinant chains or fragments thereof each comprising an IL-15Rβ signaling domain.

### Embodiment 12

The composition of embodiment 11, wherein the recombinant common γ chain or fragment thereof comprises (i) a transmembrane domain, and (ii) an intracellular portion of a common γ chain.

### Embodiment 13

The composition of any of embodiments 1-12, wherein dimerization of the recombinant chains initiates internal (IL-7R, IL-2R, or IL-15R) signaling.

### Embodiment 14

The composition of embodiment 13, wherein either:

homodimerization of i) two recombinant chains or fragments thereof each comprising an IL-7Rα endodomain or signaling domain, ii) two recombinant chains or fragments thereof each comprising an IL-2Rβ endodomain or signaling domain, or iii) two recombinant chains or fragments thereof each comprising an IL-15Rβ endodomain or signaling domain; or

heterodimerization of iv) a recombinant chain or fragment thereof comprising an IL-7Rα endodomain or signaling domain and a recombinant chain or fragment thereof comprising a common γ endodomain or signaling domain, v) a recombinant chain or fragment thereof comprising an IL-2Rβ endodomain or signaling domain and a recombinant chain or fragment thereof comprising a common γ endodomain or signaling domain, and vi) a recombinant chain or fragment thereof comprising an IL-15Rβ endodomain or signaling domain and a recombinant chain or fragment thereof comprising a common γ endodomain or signaling domain

initiates internal (IL-7R, IL-2R, or IL-15R) signaling.

### Embodiment 15

The composition of any of embodiments 1-14, wherein the drug or soluble protein comprises a recombinant human protein.

### Embodiment 16

The composition of embodiment 15, wherein the recombinant human protein is modified to alter its function.

### Embodiment 17

The composition of embodiment 16, wherein the altered function comprises modified stability, modified binding efficacy, modified natural function, modified specificity, modified immunogenicity, or modified half-life.

### Embodiment 18

The composition of any of embodiments 1-17, wherein the drug or soluble protein is chosen from: an opioid antagonist, a vitamin, a cannabinoid, an antibiotic, dihydrostreptomycin, a coxib, a profen, fenclozic acid, fenclofenac, a NDRI antidepressant, a hydrazine MAOI, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs, a formin, an antihistamine, a 5HT4 agonist, Oxyphenisatine, nefazodone, levamisole (antihelminthic), Flosequinan (quinolone vasodilator), metamizole, dimethylamylamine (DMAA, Forthane), insulin (optionally inactivated), osteopontin or a form thereof, and a monoclonal antibody.

### Embodiment 19

The composition of embodiment 18, wherein the drug or soluble protein is a monoclonal antibody, or a fragment thereof.

### Embodiment 20

The composition of embodiment 19, wherein the drug or soluble protein is omalizumab.

### Embodiment 21

A composition comprising a population of CAR-bearing immune effector cells, the cells comprising a transmembrane protein, the transmembrane protein comprising at least two protein chains, each chain comprising an endodomain, a transmembrane domain, and an ectodomain;

wherein the endodomains of the at least two chains of the transmembrane protein together comprise a structure functionally similar to that of an IL-2 receptor endodomain, an IL-7 receptor endodomain, or an IL-15 receptor endodomain; and

wherein administration of a chemical compound results in dimerization of the at least two protein chains and activates IL-2, IL-7, and/or IL-15 signaling in CAR-bearing immune effector cells.

### Embodiment 22

The composition of embodiment 21 wherein the at least two protein chains comprise:

a recombinant IL-7Rα chain or fragment thereof and a recombinant common γ chain or fragment thereof;

two recombinant IL-7Rα chains or fragments thereof;

a recombinant IL-2Rβ chain or fragment thereof and a recombinant common γ chain or fragment thereof; or

two recombinant IL-2Rβ chains or fragments thereof;

a recombinant IL-15Rβ chain or fragment thereof and a recombinant common γ chain or fragment thereof; or

two recombinant IL-15Rβ chains or fragments thereof.

### Embodiment 23

The composition of embodiment 21, wherein the recombinant IL-7Rα chain or fragment thereof, IL-2Rβ chain or fragment thereof, or IL-15Rβ chain or fragment thereof comprises (i) a transmembrane domain, and (ii) an endodomain comprising an IL-7Rα, IL-2Rβ, or IL-15Rβ signaling domain.

### Embodiment 24

The composition of embodiment 21, wherein the endodomains of the at least two chains of the transmembrane protein comprise:

a recombinant chain or fragment thereof comprising an IL-7Rα signaling domain and a recombinant chain or fragment thereof comprising a common γ signaling domain;

two recombinant chains or fragments thereof each comprising an IL-7Rα signaling domain;

a recombinant chain or fragment thereof comprising an IL-2Rβ signaling domain and a recombinant chain or fragment thereof comprising a common γ signaling domain;

two recombinant chains or fragments thereof each comprising an IL-2Rβ signaling domain;

a recombinant chain or fragment thereof comprising an IL-15Rβ signaling domain and a recombinant chain or fragment thereof comprising a common γ signaling domain; or

two recombinant chains or fragments thereof each comprising an IL-15Rβ signaling domain.

### Embodiment 25

The composition of any of embodiments 21-24, wherein:

the recombinant IL-7Rα chain(s) or fragment(s) thereof comprise(s) (i) a transmembrane domain, and (ii) a binding-protein binding domain and an endodomain or signaling domain of an IL-7Rα chain;

the recombinant IL-2Rβ chain(s) or fragment(s) thereof comprise(s) (i) a transmembrane domain, and (ii) a binding-protein binding domain and an endodomain or signaling domain of an IL-2Rβ chain; or

the recombinant IL-15Rβ chain(s) or fragment(s) thereof comprise(s) (i) a transmembrane domain, and (ii) a binding-protein binding domain and an endodomain or signaling domain of an IL-15Rβ chain.

### Embodiment 26

The composition of any of embodiments 22-25, wherein the recombinant common γ chain or fragment thereof comprises (i) a transmembrane domain, and (ii) a binding-protein binding domain and an endodomain or signaling domain of a common γ chain.

### Embodiment 27

The composition of any of embodiments 22-26, wherein the binding-protein binding domain is an FKBP binding domain.

### Embodiment 28

The composition of embodiment 27, wherein the FKBP domain is located extracellularly, intracellularly between the signaling domain and the transmembrane domain, or at the terminus of the signaling domain distal to the plasma membrane.

### Embodiment 29

The composition of any of embodiments 22-28, wherein (i) the recombinant IL-7Rα chain or fragment thereof, recombinant IL-2Rβ chain or fragment thereof, or recombinant IL-15Rβ chain or fragment thereof, or (ii) the common γ chain or fragment thereof, or (iii) both the recombinant IL-7Rα or IL-2Rβ or IL-15Rβ chain or fragment thereof and the common γ chain or fragment thereof further comprises an extracellular peptide tag.

### Embodiment 30

The composition of embodiment 29, wherein the extracellular peptide tag comprises human truncated CD34.

### Embodiment 31

The composition of any of embodiments 22-30, wherein the chemical compound comprises a dimerization agent.

### Embodiment 32

The composition of embodiment 31, wherein the dimerization agent comprises FK1012.

### Embodiment 33

The composition of any of embodiments 22-32, wherein dimerization of the at least two protein chains initiates internal (IL-7R, IL-2R, or IL-15R) signaling.

### Embodiment 34

The composition of any of embodiments 242-33, wherein the ectodomain of at least one of the at least two chains of the transmembrane protein comprises an antibody or binding portion thereof.

### Embodiment 35

The composition of embodiment 34, wherein the ectodomains of the at least two chains of the transmembrane protein each comprises an antibody or binding portion thereof.

### Embodiment 36

The composition of embodiment 34, wherein the binding portion of the antibody is chosen from a Fab, scFv, F(ab′)₂, minibody, or other binding arrangement of V_{H} and V_{L} chains or CDR-containing proteins.

### Embodiment 37

The composition of embodiment 35, wherein the binding portion of each antibody is chosen from a Fab, scFv, F(ab′)₂, minibody, or other binding arrangement of V_{H} and V_{L} chains or CDR-containing proteins.

### Embodiment 38

The composition of embodiment 36, wherein the ectodomains of the at least one of the at least two chains of the transmembrane protein comprises a scFv.

### Embodiment 39

The composition of embodiment 37, wherein the ectodomains of the at least two chains of the transmembrane protein each comprise a scFv.

### Embodiment 40

The composition of any of embodiments 34-39, wherein the antibodie(s) or binding portion(s) thereof, or the scFv(s), individually or together comprise a drug- or soluble protein-recognition domain.

### Embodiment 41

The composition of embodiment 40, wherein an epitope or epitopes of the soluble protein bind to the soluble protein-recognition domain.

### Embodiment 42

The composition of embodiment 41, wherein the binding of the transmembrane protein to the drug- or soluble protein-recognition domain and dimerization of the at least two protein chains initiates internal (IL-7R, IL-2R, or IL-15R) signaling.

### Embodiment 43

A composition comprising a population of CAR-bearing immune effector cells, the cells comprising a transmembrane protein, the transmembrane protein comprising at least two chains, each chain comprising an endodomain, a transmembrane domain, and an ectodomain;

wherein the ectodomains of the at least two chains of the transmembrane protein interact together to bind to a recombinant soluble protein comprising a first and a second domain;

wherein the first domain of the recombinant soluble protein binds to the ectodomain of one of the chains of the transmembrane protein, and the second domain of the recombinant soluble protein binds to the ectodomain of the other chain of the transmembrane protein;

wherein the endodomains of the at least two chains of the transmembrane protein together comprise a structure functionally similar to that of an IL-7 receptor endodomain or an IL-15 receptor endodomain; and

wherein binding of the recombinant soluble protein to the ectodomains activates IL-7 or IL-15 signaling in CAR-bearing immune effector cells.

### Embodiment 44

The composition of embodiment 43, wherein the ectodomains of the at least two chains of the transmembrane protein each comprise an antibody or binding portion thereof.

### Embodiment 45

The composition of embodiment 44, wherein the binding portion of each antibody is chosen from a Fab, scFv, F(ab′)₂, minibody, or other binding arrangement of V_{H} and V_{L} chains or CDR-containing proteins.

### Embodiment 46

The composition of embodiment 45, wherein the ectodomains of the at least two chains of the transmembrane protein each comprise a scFv.

### Embodiment 47

The composition of any of embodiments 43-46, wherein the antibodies or binding portions thereof, or the scFvs, together comprise a drug or recombinant protein recognition domain.

### Embodiment 48

The composition of any of embodiments 43-47, wherein the recombinant soluble protein comprises two distinct epitopes.

### Embodiment 48

The composition of embodiment 48, wherein the two distinct epitopes of the recombinant soluble protein bind to the recombinant protein recognition domain.

### Embodiment 50

The composition of embodiment 49, wherein the binding of the transmembrane protein to the recombinant soluble protein recognition domain initiates internal (IL-7R or IL-15R) signaling.

### Embodiment 51

The composition of embodiment 43-50 wherein the at least two protein chains comprise:

a recombinant IL-7Rα chain or fragment thereof and a recombinant common γ chain or fragment thereof;

two recombinant IL-7Rα chains or fragments thereof;

a recombinant IL-15Rβ chain or fragment thereof and a recombinant common γ chain or fragment thereof; or

two recombinant IL-15Rβ chains or fragments thereof.

### Embodiment 52

The composition of any of embodiments 43-51, wherein:

the first domain of the recombinant soluble protein binds to the IL-7Rα chain or fragment thereof and the second domain of the recombinant protein binds to the common γ chain or fragment thereof;

the first domain of the recombinant soluble protein binds to one IL-7Rα chain or fragment thereof and the second domain of the recombinant protein binds to the other IL-7Rα chain or fragment thereof;

the first domain of the recombinant soluble protein binds to the IL-15Rβ chain or fragment thereof and the second domain of the recombinant soluble protein binds to the common γ chain or fragment thereof; or

the first domain of the recombinant soluble protein binds to one IL-15Rβ chain or fragment thereof and the second domain of the recombinant protein binds to the other IL-15Rβ chain or fragment thereof.

### Embodiment 53

The composition of embodiment 52, wherein the recombinant IL-7Rα chain or fragment thereof or IL-15Rβ chain or fragment thereof comprises (i) a transmembrane domain, and (ii) an endodomain comprising an IL-7Rα or IL-15Rβ signaling domain.

### Embodiment 54

The composition of embodiment any of embodiments 43-53, wherein the endodomains of the at least two chains of the transmembrane protein comprise:

a recombinant chain or fragment thereof comprising an IL-7Rα endodomain and a recombinant chain or fragment thereof comprising a common γ endodomain;

two recombinant chains or fragments thereof each comprising an IL-7Rα endodomain;

a recombinant chain or fragment thereof comprising an IL-15Rβ endodomain and a recombinant chain or fragment thereof comprising a common γ endodomain; or

two recombinant chains or fragments thereof each comprising an IL-15Rβ endodomain.

### Embodiment 55

The composition of embodiment 54, wherein the recombinant common γ chain or fragment thereof comprises (i) a transmembrane domain, and (ii) an intracellular portion of a common γ chain.

### Embodiment 56

The composition of any of embodiments 21-54, wherein the (recombinant) soluble protein comprises a recombinant human protein.

### Embodiment 57

The composition of embodiment 56, wherein the recombinant human protein is modified to alter its function.

### Embodiment 58

The composition of embodiment 57, wherein the altered function comprises modified stability, modified binding efficacy, modified natural function, modified specificity, modified immunogenicity, or modified half-life.

### Embodiment 59

The composition of any of embodiments 21-58, wherein the drug or soluble protein is chosen from: an opioid antagonist, a vitamin, a cannabinoid, an antibiotic, dihydrostreptomycin, a coxib, a profen, fenclozic acid, fenclofenac, a NDRI antidepressant, a hydrazine MAOI, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs, a formin, an antihistamine, a 5HT4 agonist, Oxyphenisatine, nefazodone, levamisole (antihelminthic), Flosequinan (quinolone vasodilator), metamizole, dimethylamylamine (DMAA, Forthane), insulin (optionally inactivated), osteopontin or a form thereof, and a monoclonal antibody.

### Embodiment 60

The composition of embodiment 59, wherein the drug or soluble protein is a monoclonal antibody, or a fragment thereof.

### Embodiment 61

The composition of embodiment 60, wherein the drug or soluble protein is omalizumab.

### Embodiment 62

The composition of any of embodiments 1-61, wherein the CAR-bearing immune effector cells are chosen from CAR-T cells, CAR-iNKT cells, CAR-NK cells, CAR-macrophage cells, iPSC derived CAR-T cells, and iPSC derived CAR-NK cells.

### Embodiment 63

The composition of embodiment 62, wherein the CAR-bearing immune effector cells are CAR-T cells.

### Embodiment 64

The composition of embodiment 62, wherein the CAR binds to (targets) an antigen chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

### Embodiment 65

A method for treatment of cancer comprising:

a) administering a population of CAR-bearing immune effector cells to a patient in need thereof, wherein the CAR-bearing immune effector cells comprise a transmembrane protein of any of embodiments 1-40;

b) administering a drug or soluble protein capable of binding to the transmembrane protein;

wherein administration of the population of CAR-bearing immune effector cells and the drug or soluble enhances IL-2, IL-7, or IL-15 signaling in the patient.

### Embodiment 66

The method as recited in embodiment 65, wherein the cancer is a hematologic malignancy.

### Embodiment 67

The method as recited in embodiment 66, wherein the hematologic malignancy is a T-cell malignancy.

### Embodiment 68

The method as recited in embodiment 67, wherein the T cell malignancy is T-cell acute lymphoblastic leukemia (T-ALL).

### Embodiment 69

The method as recited in embodiment 67, wherein the T cell malignancy is non-Hodgkin's lymphoma.

### Embodiment 70

The method as recited in embodiment 67, wherein the T cell malignancy is T-cell chronic lymphocytic leukemia (T-CLL).

### Embodiment 71

The method as recited in embodiment 66, wherein the hematologic malignancy is a B-cell malignancy.

### Embodiment 72

The method as recited in embodiment 71, wherein the B-cell malignancy is diffuse large B-cell lymphoma (DLBCL).

### Embodiment 73

The method as recited in embodiment 66, wherein the hematologic malignancy is multiple myeloma.

### Embodiment 74

The method as recited in embodiment 73, wherein the hematologic malignancy is acute myeloid leukemia (AML).

### Embodiment 75

The method as recited in embodiment 65, wherein the cancer is a solid tumor.

### Embodiment 76

A recombinant transmembrane protein, the transmembrane protein comprising at least two chains, each chain comprising an endodomain, a transmembrane domain, and an ectodomain;

wherein the ectodomains of the at least two chains of the transmembrane protein interact together to bind to a drug or soluble protein;

wherein the endodomains of the at least two chains of the transmembrane protein together comprise a structure functionally similar to that of an IL-2 receptor endodomain, an IL-7 receptor endodomain, or an IL-15 receptor endodomain; and

wherein binding of the drug or soluble protein to the ectodomain activates IL-2, IL-7, or IL-15 signaling in a cell.

### Embodiment 77

The recombinant transmembrane protein of embodiment 76, wherein the ectodomains of the at least two chains of the transmembrane protein each comprise an antibody or binding portion thereof.

### Embodiment 78

The recombinant transmembrane protein of embodiment 77, wherein the binding portion of each antibody is chosen from a Fab, scFv, F(ab′)₂, minibody, or other binding arrangement of V_{H} and V_{L} chains or CDR-containing proteins.

### Embodiment 79

The recombinant transmembrane protein of embodiment 78, wherein the ectodomains of the at least two chains of the transmembrane protein each comprise a scFv.

### Embodiment 80

The recombinant transmembrane protein of any of embodiments 77-79, wherein the antibodies or binding portions thereof, or the scFvs, together comprise a drug- or soluble protein-recognition domain.

### Embodiment 81

A recombinant transmembrane protein, the protein comprising an endodomain, a transmembrane domain, and an ectodomain;

wherein the ectodomain of the receptor binds to a drug or soluble protein;

wherein the endodomain of the receptor comprises a structure functionally similar to that of an IL-7R, IL-2R, or IL-15R endodomain; and

wherein binding of the drug or soluble protein to the ectodomain is capable of activating IL-7, IL-2, or IL-15 signaling in a cell.

### Embodiment 82

The transmembrane protein of any of embodiments 76-81, wherein the soluble protein comprises two distinct epitopes.

### Embodiment 83

The transmembrane protein of embodiment 82, wherein the two distinct epitopes of the soluble protein bind to the drug or soluble protein recognition domain.

### Embodiment 84

A recombinant nucleic acid encoding the transmembrane protein of any of embodiments 76-83.

### Embodiment 85

A vector comprising the nucleic acid of embodiment 84.

### Embodiment 86

The vector of embodiment 85 wherein the vector is a lentiviral plasmid vector.

### Embodiment 87

A recombinant chimeric IL-7Rα, IL-15Rβ, IL-2Rβ, or IL-γc polypeptide chain, each comprising an endodomain, a transmembrane domain, and an ectodomain, wherein the extracellular domain is modified to comprise an scFv that recognizes and binds to an epitope of a drug or soluble protein that is not IL-7, IL-15, or IL-2.

### Embodiment 88

The recombinant chimeric IL-7Rα, IL-15Rβ, IL-2Rβ, or IL-γc polypeptide of embodiment 87, wherein the transmembrane domain of the modified IL-7Rα polypeptide chain is hybridized to the extracellular domain using a CD8 linker domain.

### Embodiment 89

A recombinant nucleic acid construct encoding the chimeric IL-7Rα, IL-15Rβ, IL-2Rβ, or IL-γc polypeptide chain of any of embodiments 87-88.

### Embodiment 90

The recombinant nucleic acid construct of embodiment 89, wherein the coding sequence of the chimeric IL-7Rα, IL-15Rβ, IL-2Rβ, or IL-γc polypeptide chain is expressed in a lentiviral plasmid.

### Embodiment 91

The recombinant nucleic acid construct of embodiment 89, wherein the construct comprises at least one element or domain selected from a Kozak sequence and a CD8a signal peptide; a human IgE CE3 extracellular domain; a CD8 linker extracellular domain; an IL-7Rα transmembrane domain; an IL-7Rα intracellular domain; a P2A-Thy1.1 co-expressed surface marker; an anti-4-Hydroxy-3-nitrophenyl (NP) 3B44 SCFV extracellular domain; and/or a CEA a2-b3 extracellular domain.

### Embodiment 92

The recombinant nucleic acid construct of embodiment 89, wherein the construct comprises at least one element or domain selected from SEQ ID NOs:1-8.

### Embodiment 93

The recombinant nucleic acid construct of embodiment 92, wherein the construct comprises:

SEQ ID NOs:1-6;

SEQ ID NOs:1 and 3-7; and/or

SEQ ID NOs:1, 3-6, and 8.

### Embodiment 94

The recombinant nucleic acid construct of any of embodiments 90-93, wherein the lentiviral plasmid vector is pLVM-EF1a.

### Embodiment 95

A host cell comprising the recombinant chimeric IL-7Rα, IL-15Rβ, IL-2Rβ, or IL-γc polypeptide of any of embodiments 87-88.

### Embodiment 96

A host cell comprising the recombinant nucleic acid construct of any of embodiments 89-94.

Embodiments of the present disclosure also provide a composition comprising a population of CAR-bearing immune effector cells, the cells comprising a transmembrane protein, the transmembrane protein comprising at least two chains, each chain comprising an endodomain, a transmembrane domain, and an ectodomain;

wherein the ectodomains of the at least two chains of the transmembrane protein interact together to bind to a drug or soluble protein;

wherein the endodomains of the at least two chains of the transmembrane protein together comprise a structure functionally similar to that of an IL-2 receptor endodomain, an IL-7 receptor endodomain, or an IL-15 receptor endodomain; and wherein binding of the drug or soluble protein to the ectodomain activates IL-2, IL-7, or IL-15 signaling in CAR-bearing immune effector cells.

In certain embodiments, the ectodomains of the at least two chains of the transmembrane protein each comprise a scFv.

In certain embodiments, the scFvs together comprise a drug or soluble protein recognition domain.

In certain embodiments, the soluble protein comprises two distinct epitopes.

In certain embodiments, the two distinct epitopes of the soluble protein bind to the drug or soluble protein recognition domain.

In certain embodiments, the binding of the transmembrane protein to the drug or soluble protein recognition domain initiates internal signaling.

In certain embodiments, the at least two chains comprise: a recombinant IL-7Rα chain or a fragment thereof and a recombinant common γ chain or a fragment thereof; a recombinant IL-2Rβ chain or a fragment thereof and a recombinant common γ chain or a fragment thereof; or a recombinant IL-15Rβ chain or a fragment thereof and a recombinant common γ chain or a fragment thereof.

In certain embodiments, the recombinant IL-7Rα chain or fragment thereof, IL-2Rβ chain or fragment thereof, or IL-15Rβ chain or fragment thereof comprises (i) a transmembrane domain, and (ii) an endodomain comprising an intracellular portion of an IL-7Rα chain, IL-2Rβ chain, or IL-15Rβ chain.

In certain embodiments, the recombinant common γ chain or fragment thereof comprises (i) a transmembrane domain, and (ii) an intracellular portion of a common γ chain.

In certain embodiments, the drug or soluble protein comprises a recombinant human protein.

In certain embodiments, the recombinant human protein is modified to alter its function.

In certain embodiments, the altered function comprises modified stability, modified binding efficacy, modified natural function, modified specificity, modified immunogenicity, or modified half-life.

In certain embodiments, the CAR-bearing immune effector cells are chosen from CAR-T cells, CAR-iNKT cells, or CAR-NK cells.

In certain embodiments, the ectodomains of the at least two chains of the transmembrane protein interact together to bind to a drug.

In certain embodiments, the drug is chosen from: an opioid antagonist, a vitamin, a cannabinoid, an antibiotic, dihydrostreptomycin, a coxib, a profen, fenclozic acid, fenclofenac, a NDRI antidepressant, a hydrazine MAOI, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs, a formin, an antihistamine, a 5HT4 agonist, Oxyphenisatine, nefazodone, levamisole (antihelminthic), Flosequinan (quinolone vasodilator), metamizole, dimethylamylamine (DMAA, Forthane), insulin (optionally inactivated), osteopontin or a form thereof, or a monoclonal antibody.

In certain embodiments, the opioid agonist is naltrexone or naloxone. In certain embodiments, the vitamin is vitamin D, vitamin C, vitamin B6. In certain embodiments, the cannabinoid is dronabinol. In certain embodiments, the antibiotic is a fluoroquinolone antibiotic. In certain embodiments, the floxacin is trovafloxacin, temafloxacin, sparfloxacin, grepafloxacin, gatifloxacin, or alatrofloxacin. In certain embodiments, the coxib is valdecoxib, rofecoxib, or lumiracoxib. In certain embodiments, the profen is suprofen or benoxaprofen. In certain embodiments, the NDRI antidepressant is nomifenisine. In certain embodiments, the hydrazine MAOI is tranylcypromine. In certain embodiments, the PPAR is troglitazone. In certain embodiments, the formin is phenformin or buformin. In certain embodiments, the antihistamine is terfenadine or thenalidine. In certain embodiments, the 5HT4 agonist is tegaserod maleate.

In certain embodiments, the ectodomains of the at least two chains of the transmembrane protein interact together to bind to a soluble protein.

In certain embodiments, the soluble protein is chosen from insulin or osteopontin, or an analogue of either of the foregoing.

In certain embodiments, the soluble protein is insulin.

In certain embodiments, the insulin is inactivated such that it does not bind to and/or activate the insulin receptor or otherwise modulate metabolic effects or act as a hormone. In certain embodiments, the insulin is non-immunogenic. In certain embodiments, the insulin is substantially human.

In certain embodiments, the soluble protein is osteopontin.

In certain embodiments, the osteopontin is chosen from OPN-a, OPN-b, OPN-c, osteopontin-L, osteopontin-R, and osteopontin-166.

In certain embodiments, the soluble protein is a monoclonal antibody.

In certain embodiments, the monoclonal antibody is chosen from Adalimumab (Amjevita®), Bezlotoxumab (Zinplava™), Avelumab (Bavencio®), Dupilumab (Dupixent®), Durvalumab (Imfinzi®), Ocrelizumab (Ocrevus™), Brodalumab (Siliq), Reslizumab (Cinqair™), Olaratumab (Lartruvo), Daratumumab (Darzalex®), Elotuzumab (Empliciti), Necitumumab (Portrazza), Infliximab (Inflectra), Obiltoxaximab (Anthim®), Atezolizumab (Tecentriq®), Secukinumab (Cosentyx™), Mepolizumab (Nucala), Nivolumab (Opdivo), Alirocumab (Praluent), Idarucizumab (Praxbind®), Evolocumab (Repatha®), Dinutuximab (Unituxin), Bevacizumab (Blincyto®), Pembrolizumab (Keytruda®), Ramucirumab (Cyramza), Vedolizumab (Entyvio®), Siltuximab (Sylvant®), Alemtuzumab (Lemtrada®), Trastuzumab emtansine (Kadcyla®), Pertuzumab (Perjeta®), Infliximab (Remsima®), Obinutuzumab (Gazyvaro®), Brentuximab (Adcetris®), Raxibacumab (ABthrax®), Belimumab (Benlysta®), Ipilimumab (Vervoy®), Denosumab (Xgeva®), Denosumab (Prolia®), Ofatumumab (Arzerra®), Besilesomab (Scintimun®), Tocilizumab (RoActemra®), Canakinumab (Ilaris®), Golimumab (Simponi®), Ustekinumab (Stelara®), Certolizumab pegol (Cimzia®), Catumaxomab (Removab®), Eculizumab (Soliris®), Ranibizumab (Lucentis®), Panitumumab (Vectibix®), Natalizumab (Tysabri®), Catumaxomab (Proxinium®), Bevacizumab (Avastin®), Omalizumab (Xolair®), Cetuximab (Erbitux®), Efalizumab (Raptiva®), Ibritumomab tiuxetan (Zevalin®), Fanolesomab (NeutroSpec®), Adalimumab (Humira®), Tositumomab and iodine 131 tositumomab (Bexxar®), Alemtuzumab (Campath®), Trastuzumab (Herceptin®), Gemtuzumab ozogamicin (Mylotarg®), Infliximab (Remicade®), Palivizumab (Synagis®), Necitumumab (Daclizumab), Basiliximab (Simulect®), Rituximab (Rituxan® MabThera®), Votumumab (Humaspect®), Sulesomab (LeukoScan®), Arcitumomab (CEA-scan®), Imiciromab (MyoScint®), Capromab (ProstaScint®), Nofetumomab (Verluma®), Abciximab (ReoPro®), Satumomab (OncoScint®), and/or Muromonab-CD3 (Orthoclone OKT3®).

In certain embodiments, the monoclonal antibody is omalizumab.

Also provided herein is a composition comprising a population of CAR-bearing immune effector cells, the cells comprising a transmembrane protein, the transmembrane protein comprising at least two protein chains, each chain comprising an endodomain, a transmembrane domain, and an ectodomain;

wherein the endodomains of the at least two chains of the transmembrane protein together comprise a structure functionally similar to that of an IL-2 receptor endodomain, an IL-7 receptor endodomain, or an IL-15 receptor endodomain; and

wherein administration of a chemical compound results in dimerization of the at least two protein chains and activates IL-2, IL-7, and/or IL-15 signaling in CAR-bearing immune effector cells.

Also provided herein are embodiments comprising the combination of a modified common gamma chain or fragment thereof and modified IL-7Rα chain or fragment thereof and/or modified IL-2Rβ chain or fragment thereof and/or modified IL-15Rβ chain or fragment thereof, such that signaling may induce both IL-7 and/or IL-2 and/or IL-15 signaling (the common gamma chain would be able to dimerize with both modified IL-7Rα chain or fragment thereof and/or modified IL-2Rβ chain or fragment thereof and/or modified IL-15Rβ chain or fragment thereof to induce both IL-7 and IL-2, and/or IL-7 and IL-15, and/or IL-2 and IL-15 signaling and combinations thereof.

In certain embodiments, the at least two protein chains comprise: a recombinant IL-7Rα chain or fragment thereof and a recombinant common γ chain or fragment thereof; a recombinant IL-2Rβ chain or fragment thereof and a recombinant common γ chain or fragment thereof; or a recombinant IL-15Rβ chain or fragment thereof and a recombinant common γ chain or fragment thereof.

In certain embodiments: the recombinant IL-7Rα chain comprises (i) a transmembrane domain, and (ii) an endodomain comprising a binding-protein binding domain (such as an FKBP binding domain) and an intracellular portion of an IL-7Rα chain; the recombinant IL-2Rβ chain comprises (i) a transmembrane domain, and (ii) an endodomain comprising an FKBP binding domain and an intracellular portion of an IL-2Rβ chain; or the recombinant IL-15Rβ chain comprises (i) a transmembrane domain, and (ii) an endodomain comprising an FKBP binding domain and an intracellular portion of an IL-15Rβ chain.

In certain embodiments, the recombinant common γ chain comprises (i) a transmembrane domain, and (ii) an endodomain comprising an FKBP binding domain and an intracellular portion of a common γ chain.

In certain embodiments, the FKBP domain is located extracellularly or at the terminal of the intracellular signaling domain distal to the plasma membrane.

In certain embodiments, (i) the recombinant IL-7Rα chain or fragment thereof, recombinant IL-2Rβ chain or fragment thereof, or recombinant IL-15Rβ chain or fragment thereof, or (ii) the common γ chain or fragment thereof, or (iii) both the recombinant IL-7Rα chain or fragment thereof and the common γ chain or fragment thereof further comprises an extracellular peptide tag.

In certain embodiments, the extracellular peptide tag comprises human truncated CD34.

In certain embodiments, the chemical compound comprises a dimerization agent.

In certain embodiments, the dimerization agent comprises FK1012.

In certain embodiments, dimerization of the at least two protein chains initiates internal signaling.

In certain embodiments, the CAR-bearing immune effector cells are chosen from CAR-T cells, CAR-iNKT cells, or CAR-NK cells.

Also provided is a method for treatment of cancer comprising: a) administering a population of CAR-bearing immune effector cells to a patient in need thereof, wherein the CAR-bearing immune effector cells comprise a transmembrane protein of any of the embodiments disclosed herein; b) administering a drug or soluble protein capable of binding to the transmembrane protein; wherein administration of the population of CAR-bearing immune effector cells and the drug or soluble enhances IL-2, IL-7, or IL-15 signaling in the patient.

Also provided herein is a recombinant transmembrane protein, the protein comprising an endodomain, a transmembrane domain, and an ectodomain; wherein the ectodomain of the receptor binds to a drug or soluble protein; wherein the endodomain of the receptor comprises a structure functionally similar to that of an IL-7R, IL-2R, or IL-15R endodomain; and wherein binding of the drug to the ectodomain is capable of activating IL-7, IL-2, or IL-15 signaling in a cell.

Also provided herein is a recombinant nucleic acid encoding the transmembrane protein disclosed herein.

Also provided herein is a composition comprising a population of CAR-bearing immune effector cells, the cells comprising a transmembrane protein, the transmembrane protein comprising at least two chains, each chain comprising an endodomain, a transmembrane domain, and an ectodomain;

wherein the ectodomains of the at least two chains of the transmembrane protein interact together to bind to a recombinant protein comprising a first and a second domain;

wherein the first domain of the recombinant protein binds to the ectodomain of one of the chains of the transmembrane protein, and the second domain of the recombinant protein binds to the ectodomain of the other chain of the transmembrane protein;

wherein the endodomains of the at least two chains of the transmembrane protein together comprise a structure functionally similar to that of an IL-7 receptor endodomain or an IL-15 receptor endodomain; and

wherein binding of the recombinant protein to the ectodomains activates IL-7 or IL-15 signaling in CAR-bearing immune effector cells.

In certain embodiments, the ectodomains of the at least two chains of the transmembrane protein each comprise a scFv.

In certain embodiments, the scFvs together comprise a recombinant protein recognition domain.

In certain embodiments, the soluble protein comprises two distinct epitopes.

In certain embodiments, the two distinct epitopes of the recombinant protein bind to the recombinant protein recognition domain.

In certain embodiments, the binding of the transmembrane protein to the recombinant protein recognition domain initiates internal signaling.

In certain embodiments, the at least two chains comprise: a recombinant IL-7Rα chain or fragment thereof and a recombinant common γ chain or fragment thereof; or a recombinant IL-15Rβ chain or fragment thereof and a recombinant common γ chain or fragment thereof.

In certain embodiments, the first domain of the recombinant protein binds to the IL-7Rα chain or fragment thereof and the second domain of the recombinant protein binds to the common γ chain or fragment thereof; or the first domain of the recombinant protein binds to the IL-15Rβ chain or fragment thereof and the second domain of the recombinant protein binds to the common γ chain or fragment thereof.

In certain embodiments, the recombinant IL-7Rα chain or fragment thereof, or IL-15Rβ chain or fragment thereof comprises (i) a transmembrane domain, and (ii) an endodomain comprising an intracellular portion of an IL-7Rα chain, or IL-15Rβ chain.

In certain embodiments, the recombinant common γ chain comprises (i) a transmembrane domain, and (ii) an intracellular portion of a common γ chain.

In certain embodiments, the recombinant protein comprises a recombinant human protein.

In certain embodiments, the recombinant human protein is modified to alter its function.

In certain embodiments, the altered function comprises modified stability, modified binding efficacy, modified natural function, modified specificity, modified immunogenicity, or modified half-life.

In certain embodiments, the CAR-bearing immune effector cells are chosen from CAR-T cells, CAR-iNKT cells, or CAR-NK cells.

Interleukin-7 (IL-7) and its Receptor IL-7R

Interleukin-7 (IL-7) is a cytokine with a central role in the adaptive immune system. It promotes lymphocyte development in the thymus and maintains survival of naive and memory T cell homeostasis in the periphery. IL-7 is also important for the organogenesis of lymph nodes and for the maintenance of activated T cells.

Preclinical models have shown that administration of IL-7 to patients following administration of CAR-T cell therapy promotes expansion and survival of administered T cells. However, while cytokines have been applied in cancer immunotherapy for many years, the high doses required to achieve a significant clinical response in patients results in serious complications, such as fever, severe biochemical abnormalities in the liver and kidney, and capillary leak, collectively known as cytokine release syndrome (CRS). In addition, constitutively active IL-7R, or CAR-T cells that secrete IL-7, are not able to be controlled or regulated once administered to a patient. The present disclosure provides the ability to modulate IL-7R signaling as deemed appropriate and to mitigate CRS, improve safety, and enhance efficacy.

A patient or subject requiring protection from a disease such as cancer requires an adequate supply of T lymphocytes, which are maintained in the body in a dynamic balance. Antigen-specific effector T cells die after performing their cellular function, followed by expansion of new T cells to replace them. IL-7 functions to maintain this balance of T cells. As IL-7 is involved in a myriad of cellular functions, excess IL-7 in circulation can result in a number of effects in a subject. Therefore, while IL-7 signaling in the body provides many functions, excess IL-7 in the circulation may be less beneficial. Thus, the present disclosure provides a modified IL-7 receptor that does not recognize or bind to IL-7, eliminating the need for administration of high doses of IL-7. Instead, a modified IL-7 receptor as described herein binds to a drug, such as a drug for treatment of cancer. Such a modified IL-7 receptor activates IL-7 signaling upon binding of a drug without requiring IL-7 binding, effectively avoiding off-target effects occurring with excess IL-7 in circulation.

The human IL-7 gene is found on chromosome 8q12-13, and is 72 kb in length, encoding a protein of 177 amino acids with a molecular weight of 20 kDa. IL-7 exerts its effects by, first, binding to its natural receptor, i.e., IL-7R. The IL-7R gene is composed of 8 exons, producing a transcript of 4619 bp. Alternative splicing creates a soluble isoform of the receptor that lacks exon 6 by introducing a premature stop codon. The receptor of IL-7, i.e., IL-7R, is a heterodimer that consists of two chains: IL-7Rα (CD127), which is shared with thymic stromal lymphopoietin (TSLP), and the common γ chain (CD132), which is shared among IL-2, IL-4, IL-9, IL-15, and IL-21. The γ chain is expressed on all hematopoietic cell types, while IL-7Rα is mainly expressed by lymphocytes, including common T/B lymphoid precursors, developing T and B cells, naïve T cells, and memory T cells. IL-7Rα is also found in innate lymphoid cells, such as natural killer (NK) cells and gut-associated lymphoid tissue (GALT)-derived LTi cells, which have critical functions in lymphoid organ development and innate immune responses to pathogens. Mutations in IL-7Rα result in severe combined immune deficiency (SCID) in humans caused by a lack of T cells.

IL-7Rα is regulated by transcription factors GABPα and Foxo1, which stimulate the activity of the receptor, as well as the inhibitory transcription factor Gfi-1, the expression of which is inhibited by TGF-β, thereby promoting IL-7Rα expression. In addition, soluble IL-7R, which competes with cell membrane-bound IL-7R to reduce excessive IL-7 consumption by IL-7R-expressing target cells, enhances the bioactivity of IL-7 when the cytokine is limited.

IL-7 Signaling

IL-7 signaling occurs when IL-7 in circulation binds to its cellular receptor (IL-7R). There are two main intracellular signaling pathways responsible for the function of IL-7: Jak-Stat and PI3K-Akt. The cytosolic tail of the IL-7Rα chain is associated with the protein tyrosine kinase Janus kinase 1 (Jak1), and the cytosolic tail of the γ chain is associated with Jak3. Binding of IL-7 to its receptor causes activation of Jak in the cytosol, phosphorylating signal transducer and activator of transcription (STAT) proteins. The dimeric phosphorylated STAT (pSTAT) proteins subsequently translocate into the nucleus to activate gene expression. IL-7 activates the anti-apoptotic genes Bcl-2 and Mcl-1 via the Jak3-Stat5 pathway, leading to suppression of pro-apoptotic proteins, such as Bax and Bak. The inhibition of these apoptotic pathways results in the survival of naïve and memory T cells. This function is IL-7 dose-dependent, i.e., a higher concentration of IL-7 induces proliferation of T cells, while lower concentrations of IL-7 sustain cell survival. Stimulation of CAR-T cells with IL-7 during their manufacture induces their expansion and proliferation; so does co-expression of IL-7 (Huang et al., Gene Therapy 25:192-197, 2018).

Binding of IL-7 to IL-7R activates multiple pathways that regulate lymphocyte survival, glucose uptake, proliferation, and differentiation.

There are very limited amounts of IL-7 under physiological conditions in vivo. Stromal cells produce IL-7 at relatively constant amounts, independent of external stimuli. Regulation of the function of IL-7 relies on the levels of the IL-7Rα chain. In addition, binding of IL-7 downregulates IL-7Rα expression by decreasing its gene transcription. IL-7Rα is highly expressed on naïve and central memory T cells. Once primed by recognition of an antigen, naïve T cells differentiate into effector T cells and lose IL-7Rα expression such that more naïve T cells obtain limited IL-7 to survive and proliferate. IL-7Rα is then re-expressed when effector T cells differentiate to the memory stage. It is therefore apparent that IL-7 levels in the cell affect many different cellular pathways.

While cytokines have been applied in cancer immunotherapy for many years, the high doses required to achieve a significant clinical response in patients can result in serious complications, such as fever, severe biochemical abnormalities in the liver and kidney, and capillary leak (e.g., cytokine release syndrome). A patient or subject requiring protection from a disease such as cancer requires an adequate supply of T lymphocytes, which are maintained in the body in a dynamic balance. Antigen-specific effector T cells die after performing their cellular function, followed by expansion of new T cells to replace them. IL-7 functions to maintain this balance of T cells. As IL-7 is involved in a myriad of cellular functions, excess IL-7 in circulation can result in a number of effects in a subject. Therefore, while IL-7 signaling in the body provides many functions, excess IL-7 in the circulation may be less beneficial.

Thus, the present disclosure provides a modified IL-7 receptor that does not recognize or bind to IL-7, eliminating the need for administration of high doses of IL-7. Instead, a modified IL-7 receptor as described herein binds to a drug, such as a drug for treatment of cancer. Such a modified IL-7 receptor activates IL-7 signaling upon binding of a drug without requiring IL-7 binding, effectively avoiding off-target effects occurring with excess IL-7 in circulation.

Patients having had high dose chemotherapy and auto-immune diseases often have increased circulating levels of IL-7. Higher levels of IL-7 induce T cell homeostasis proliferation outside of the thymus. After the recovery of CD4⁺ T cell populations, IL-7 returns to homeostatic levels due to naive CD4⁺ T cells expression of IL-7R, which consumes IL-7 to maintain their survival. However, IL-7 has more potent effects on CD8⁺ T cells compared with CD4⁺ T cells. In addition, CD8⁺ T cells survive better and proliferate faster than CD4⁺ T cells. Furthermore, IL-7 signaling on IL-7Rα⁺ DCs downregulates the expression of MHC class II on DCs, which is critical for the expansion of CD4⁺ T cells. During CD8⁺ T cell homeostasis, IL-7 signaling must be intermittent due to continuous IL-7 signaling results in increased pSTAT5, which can activate IFN-γ gene expression and cell apoptosis triggered by IFN-γ. The intermittent interruption of IL-7R signaling is induced by TCR stimulation, which is dependent on the calcium-sensitive protease calpain to cleave the cytosolic tail of the γ chain and dissociate Jak3 from IL-7R. The TCR signal also acutely reduces Jak1 protein levels and prevents its synthesis to impair IL-7 signaling via microRNA-17.

Interleukin-2 (IL-2) and its Receptor IL-2R

The interleukin-2 receptor (IL-2R) is a heterotrimeric protein expressed on the surface of immune cells, such as lymphocytes, including lymphoid linages T, B, and NK cells, including CD8+ T cells, regulatory T (Treg) cells, among others, as well as myeloid ones like macrophages, monocytes, and neutrophils. IL-2R binds to the cytokine IL-2. IL-2R can have three forms, based on different combinations of three different protein chains, IL-2Rα (also known as CD25), IL-2Rβ (also known as CD122), and IL-2Rγ (also known as the common gamma chain, or CD132), which is also common to the IL-7R. IL-2Rβ and IL-2Rγ chains of the IL-2R are members of the type I cytokine receptor family. The three IL-2R receptor chains are expressed separately and differently on various cell types and can assemble in different combinations and orders to generate low, intermediate, and high affinity IL-2 receptors.

The IL-2Rα chain binds IL-2 with low affinity, while the combination of IL-2Rβ and IL-2Rγ together form a complex that binds IL-2 with intermediate affinity. This arrangement is primarily found on memory T cells and NK cells. All three receptor chains form a complex that binds IL-2 with high affinity (Kd˜10-11 M) on activated T cells and regulatory T cells. The intermediate and high affinity receptor forms are functional and cause changes in the cell upon IL-2 binding.

Structural studies using X-ray crystallography indicate that IL-2 initially binds to the high affinity receptor at the IL-2Rα chain, which results in sequential recruitment of IL-2Rβ and then IL-2Rγ.

Interleukin-15 (IL-15) and its Receptor IL-15R

The interleukin-15 receptor (IL-15R) is a type I cytokine receptor that binds IL-15. IL-15R is made up of the IL-15Rα chain, which is homologous to the IL-2Rα chain, the β chain (CD122), which is shared with IL-2R, and the common γ chain, which is shared with both IL-7R and IL-2R. IL-15R is expressed in T cells, NK cells, NKT cells, dendritic cells, and monocytes.

The IL-15Rα has a particularly high affinity for IL-15 in comparison with all other cytokine receptors, due to an unusual structure of the IL-15RA chain having an additional region called the sushi domain. The action on the sushi-domain receptor (IL-15Rα) is transduced into the cell via the Jak1 and Jak3 kinases that phosphorylate STAT-3, STAT-5, and STAT-6 nuclear factors and, with the help of some mitogen-activated kinases (MAPK), result in enhanced transcription of IL-15-dependent genes in the cell nucleus.

Given the shared receptor usage between IL-2R and IL-15R, these two receptors exhibit similar effects on lymphoid cells. Like IL-2, IL-15 induces proliferation and cytokine production in T and NK cells. However, IL-15 is less efficient than IL-2 in inducing effector memory T-cell differentiation or sensitivity to apoptosis. IL-15Rα is more widely expressed than IL-2Rα, IL-2Rβ, and the common γ chain. IL-15Rα is expressed by lymphoid cells, DCs, fibroblasts, and epithelial, liver, intestine, and other cells and is thought to present IL-15 in trans to cells expressing IL-15β and γ chains.

Modification of IL-7, IL-2, and IL-15 Receptors (IL-7R, IL-2R, IL-15R)

As described herein, the disclosure provides a modified IL-7R, IL-2R, or IL-15R that is capable of binding to a drug or soluble protein and activating IL-7, IL-2, or IL-15 signaling. Exemplary modified or recombinant IL-7 receptors are provided herein. Similar modified or recombinant IL-2 and IL-15 receptors can be similarly made.

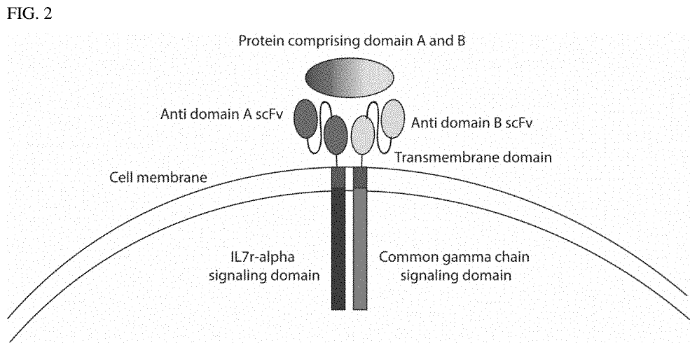

As shown in FIG. 2, a recombinant IL-7Rα chain may comprise a domain A scFv, linked to a transmembrane domain (including, but not limited to, a transmembrane domain of IL-7Rα or CD28), which in turn may be coupled to an intracellular portion of an IL-7Rα chain. A recombinant common γ chain may comprise a domain B scFv linked to a transmembrane domain (including, but not limited to, a transmembrane domain of the common γ chain or CD28), which in turn may be coupled to an intracellular portion of a common γ chain.

A recombinant soluble protein comprising both domain A and domain B, which bind to domain A scFv and domain B scFv, may be a recombinant human soluble protein, which may be deactivated such that it does not exhibit natural function, and which may additionally be modified to increase stability, binding efficacy, inhibition of natural functions, specificity, immunogenicity, and/or half-life. Examples of such human proteins may include, but are not limited to insulin and osteopontin. The recombinant soluble protein may also be an antibody, or fragment thereof, such as omalizumab.

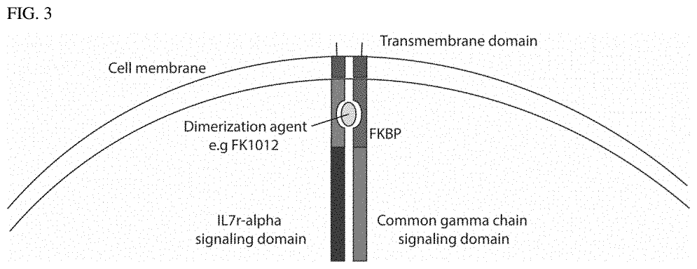

As shown in FIG. 3, administration of a chemically induced dimerization agent may lead to dimerization of the IL-7Rα chain and the common γ chain, thus inducing IL-7R signaling. Such dimerization agents may include, but are not limited to, FK1012, FK506, FKCsA, rapamycin, coumermycin, gibberellin, HaXS, TMP-HTag, ABT-737, or the like.

A recombinant IL-7Rα chain may comprise a transmembrane domain (including, but not limited to, a transmembrane domain of IL-7Rα or CD28), linked to, for example, an FKBP binding domain, or other appropriate binding domain known and/or available in the art, which in turn may be coupled to an intracellular portion of an IL-7Rα chain. The recombinant common gamma chain may comprise a transmembrane domain (including, but not limited to, a transmembrane domain of a common gamma chain or CD28), linked to an FKBP binding domain, which in turn may be coupled to an intracellular portion of a common γ chain. In some embodiments, the recombinant IL-7Rα chain and/or the common γ chain may also include an extracellular peptide tag, for example, human truncated CD34. In some embodiments, the position of an FKBP domain may alternatively be located extracellularly, or at the terminal of the intracellular signaling domain distal to the plasma membrane.

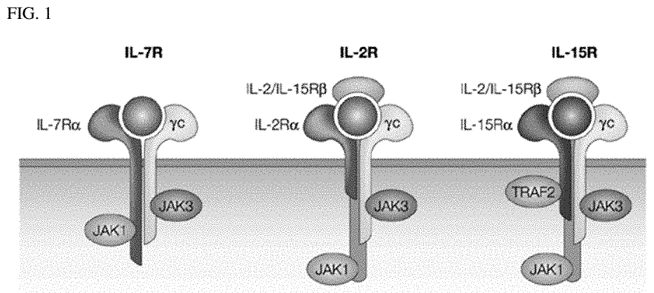

Any modifications to an IL-7R as described herein or encompassed within the scope of the disclosure may be accomplished by modifying one or more polypeptide chains of the IL-7R. The structure of the IL-7R is well known in the art and shown in FIG. 1. For example, as described herein, IL-7R is composed of two different protein chains (heterodimer), an alpha chain (IL-7Rα or CD127), which has a molecular weight (MW) of 75 kDa, and a common gamma chain (γ_{c} or CD132), which is shared with various other cytokines (i.e., IL-2, 4, 9, 15, and 21). In some embodiments, effective modification of the IL-7R may be at the IL-7Rα chain, which is specific to IL-7R, and therefore modifications to this chain would specifically affect IL-7 and have no effect on other cytokines. In some embodiments, modification of the IL-7Rα chain may be at the extracellular domain, which is where the IL-7 ligand binds to initiate downstream IL-7 signaling.

In some embodiments, modifications to an IL-7R may be made in the alpha chain (IL-7Rα), or the common gamma (γc) chain. In some embodiments, preferred modifications may be made in the alpha chain for specificity to IL-7 signaling pathways in a cell. Modifications may be made to the nucleic acid sequence of the IL-7R. In some embodiments, modifications may be made to the DNA sequence such that a drug recognition domain is introduced into the ectodomain of the IL-7R, resulting in the ectodomain recognizing and/or binding to a drug as described herein. An ectodomain of the present disclosure may recognize a drug as described herein below, or may recognize multiple drugs. In some embodiments, a single ectodomain may be introduced into a modified IL-7R of the disclosure such that the ectodomain may recognize a class of drugs as described herein, such as a drug for treatment of cancer. Such modifications may require changes in the nucleic acid sequence of an IL-7R in order to produce a modified IL-7R as described herein. One of skill in the art will understand that due to the degeneracy of the genetic code, a number of changes in the nucleic acid sequence of an IL-7R may result in the same IL-7R protein sequence, and these alternative nucleic acid sequences are intended to be encompassed within the scope of the disclosure. Any specific modifications to the sequence of an IL-7R may be made, as long as the produced protein sequence is capable of binding to a drug of interest as described herein, and as long as the produced protein, when expressed in a cell as described herein, exhibits the desired function, i.e., the ability to activate IL-7 signaling pathways in the cell in which the protein is expressed. In this way, the produced modified IL-7R may be referred to herein as functionally similar to a native IL-7R. In some embodiments, when modifying the nucleic acid sequence of an IL-7R, single nucleotide changes may be made, or multiple nucleotide changes may be made, all of which are encompassed within the scope of the present disclosure. As described herein, modification may be made using any methods known and/or available in the art.

In some embodiments, the disclosure provides modified IL-2 or IL-15 receptors. These receptors are described herein above and may be modified according to methods as described herein. As described for IL-7R, modifications of IL-2R and/or IL-15R may be made in any of the subunit receptor chains, including, but not limited to IL-2Rβ, IL-15Rβ, and/or γc chains.

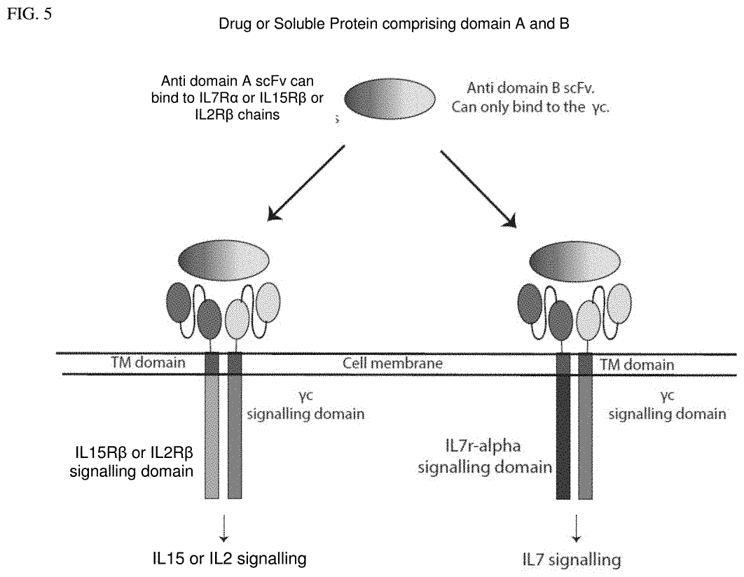

In some embodiments, a modified IL-7 or IL-15 receptor may be bound by a single recombinant protein such that this recombinant protein is able to bind to either the IL-7 or IL-15 receptors. Production of a recombinant protein may involve any methods known or available in the art. In some embodiments, a recombinant protein may be engineered to have separate domains, each of which recognizes a different, distinct ectodomain on a modified receptor of the present disclosure. An exemplary receptor demonstrating this is provided in FIG. 5, in which a recombinant protein having domains A and B is able to bind to the ectodomains of either IL-7R or IL-15R as described herein. As shown in the figure, an antidomain A scFv of such a recombinant protein may bind to either the IL-15Rβ or the IL-7Rα chains as described herein, but is not able to bind to the common γ chain. Likewise, antidomain B of such a recombinant protein may bind to the common γ chain, but is not able to bind to the IL-15Rβ or the IL-7Rα. Thus, a recombinant protein having domains A and B is capable of binding to either the IL-7R or the IL-15R at domain A, as well as the common γ chain at domain B, thereby activating signaling from either the IL-7R or the IL-15R. A protein having only domain A or only domain B would not be able to bind both ectodomains of each chain of a modified receptor as described herein and would therefore not activate IL-7 or IL-15 signaling in the cell.

In some embodiments, homodimerization of an IL-7Rα chain as described herein may induce IL-7 signaling in a cell without the requirement of the common γ chain. This can be achieved in two ways. First, homodimerization of two identical IL-7Rα chains may be induced using chemical means as described herein. Any chemical useful for inducing this can be used as described herein. Second, an scFv-IL-7Rα fusion may be created as described herein such that both chains of the modified receptor are identical. In such a case, the modified homodimeric receptor will bind to a protein that contains two identical epitopes.

Isolation of Coding Sequences

As described herein, a modified IL-7 receptor is provided that recognizes and binds to a drug or soluble protein, rather than IL-7, in order to activate or initiate IL-7 signaling in a cell. Such a modified IL-7 receptor may be produced from a native mammalian IL-7 receptor protein sequence, for example, a human IL-7 receptor protein sequence. In order to obtain the coding sequence of an IL-7R, a cDNA sequence encoding IL-7R may be isolated from a recombinant DNA library generated using either genomic DNA or cDNA. In some embodiments, a cDNA library may be constructed from polyadenylated mRNA obtained from a particular desired cell line that expresses a mammalian IL-7R. For example, the cDNA library may be constructed from cell lines that express IL-7R such as including, but not limited to human cell lines, murine cell lines, or any appropriate cell lines known and/or available in the art.

IL-7R sequences contained in a cDNA library may readily be identified by screening the library with an appropriate nucleic acid probe capable of hybridizing with IL-7R cDNA. Alternatively, DNAs encoding IL-7R proteins may be assembled by ligation of synthetic oligonucleotide subunits to obtain a complete coding sequence.

As described herein, cDNAs encoding IL-7R may be isolated by direct expression. A cDNA library may be constructed by first isolating cytoplasmic mRNA from a desired cell line. Polyadenylated RNA may be isolated and used to prepare double-stranded cDNA. Purified cDNA fragments may then be ligated into a vector as described herein in the Examples. Vectors containing the IL-7R cDNA fragments may then be transformed into an appropriate host, such as E. coli strain DH5α. Transformants may be plated to provide approximately 1,000 colonies per plate. The resulting colonies may be harvested and each pool used to prepare plasmid DNA for transfection into a desired cell line, such as described by Cosman et al. (Nature 312:768, 1984) and Luthman et al. (Nucl Acid Res 11:1295, 1983). Transfectants expressing biologically active cell surface IL-7 receptors may be identified by appropriate screening methods to identify positive foci for IL-7 binding. Bacteria from any positive pools may be grown in culture and plated to provide individual colonies, which may be screened to identify a single clone. Positive clones may be isolated, and its insert sequenced to determine the nucleotide sequence of the human IL-7R cDNA. The sequence of human IL-7R cDNA clone isolated as described may then be used as a hybridization probe to isolate cDNA clones from appropriate libraries. Using analogous methods, cDNA clones may also be isolated from cDNA libraries of other mammalian species by cross-species hybridization. For use in hybridization, DNA encoding IL-7R may be covalently labeled with a detectable substance such as a fluorescent group, a radioactive atom or a chemiluminescent group by methods well known to those skilled in the art. Such probes could also be used for in vitro diagnosis of particular conditions.

Like most mammalian genes, mammalian IL-7 receptors may be encoded by multi-exon genes. Alternative mRNA constructs that may be attributed to different mRNA splicing events following transcription, and that share large regions of identity or similarity with the cDNAs claimed herein, are considered to be within the scope of the present disclosure.

IL-7, IL-2, and IL-15 Receptor Proteins and Analogs Thereof

The present disclosure provides substantially homogeneous recombinant mammalian IL-7R, IL-2R, and IL-15R polypeptides and, optionally, without associated native-pattern glycosylation. Mammalian IL-7R, IL-2R, and IL-15R of the present disclosure may include, for example, primate, human, murine, canine, feline, bovine, ovine, equine, and/or porcine IL-7R, IL-2R, and IL-15R receptor, along with biologically active fragments thereof. Derivatives of IL-7R, IL-2R, and IL-15R within the scope of the disclosure may also include various structural forms of the primary protein that retain biological activity. Due to the presence of ionizable amino and carboxyl groups, for example, an IL-7R, IL-2R, and IL-15R protein may be in the form of acidic or basic salts, or may be in neutral form. Individual amino acid residues may also be modified by oxidation or reduction.

The primary amino acid structure of a modified IL-7R, IL-2R, and IL-15R protein may be modified by forming covalent or aggregative conjugates with other chemical moieties, such as glycosyl groups, lipids, phosphate, acetyl groups and the like, or by creating amino acid sequence mutants. Covalent derivatives may be prepared by linking particular functional groups to IL-7R, IL-2R, and IL-15R protein amino acid side chains and/or at the N- or C-termini. Other derivatives of IL-7R, IL-2R, and IL-15R encompassed within the scope of the present disclosure may also include covalent or aggregative conjugates of IL-7R, IL-2R, and IL-15R or fragments thereof with other proteins or polypeptides, such as by synthesis in recombinant culture as N-terminal or C-terminal fusions. For example, the conjugated peptide may be a signal (or leader) polypeptide sequence at the N-terminal region of the protein that co-translationally or post-translationally directs transfer of the protein from its site of synthesis to its site of function inside or outside of the cell membrane or wall (e.g., the yeast α-factor leader). IL-7R, IL-2R, and IL-15R protein fusions may comprise peptides added to facilitate purification or identification of IL-7 receptor (e.g., poly-His). The amino acid sequence of IL-7R, IL-2R, and IL-15R may also be linked to a peptide, for example a peptide having the sequence Asp-Tyr-Lys-Asp-Asp-Asp-Lys (DYKDDDDK) (Hopp et al., Bio/Technology 6:1204, 1988) or having the sequence Gly-Gly-Gly-Gly-Ser (GGGGS), among others. Many such sequences are known and may be used in accordance with the disclosure. See, for example, Li et al., Appl Microbiol and Biotechnol 100(1):215-225, 2016).

The latter sequence is highly antigenic and provides an epitope reversibly bound by a specific monoclonal antibody, enabling rapid assay and facile purification of expressed recombinant protein. This sequence is also specifically cleaved by bovine mucosal enterokinase at the residue immediately following the Asp-Lys pairing. Fusion proteins capped with this peptide may also be resistant to intracellular degradation in E. coli.

The present disclosure also provides a modified IL-7R, IL-2R, and IL-15R with or without associated native-pattern glycosylation. IL-7R, IL-2R, and IL-15R expressed in yeast or mammalian expression systems, e.g., COS-7 cells, may be similar or slightly different in molecular weight and glycosylation pattern than the native molecules, depending upon the expression system. Expression of IL-7R, IL-2R, and IL-15R DNA in bacteria such as E. coli provides non-glycosylated molecules. Functional mutant analogs of mammalian IL-7R, IL-2R, and IL-15R having inactivated N-glycosylation sites may be produced by oligonucleotide synthesis and ligation or by site-specific mutagenesis techniques, which are known and available in the art. These analog proteins can be produced in a homogeneous, reduced-carbohydrate form in good yield using yeast expression systems. N-glycosylation sites in eukaryotic proteins are characterized by the amino acid triplet Asn-A1-Z, where A1 is any amino acid except Pro, and Z is Ser or Thr. In this sequence, asparagine provides a side chain amino group for covalent attachment of carbohydrate. Such a site can be eliminated by substituting another amino acid for Asn or for residue Z, deleting Asn or Z, or inserting a non-Z amino acid between A1 and Z, or an amino acid other than Asn between Asn and A1.

IL-7 receptor derivatives may also be obtained by mutations of IL-7 receptor or subunits thereof. An IL-7 receptor mutant, as referred to herein, is a polypeptide homologous to IL-7 receptor, but which has an amino acid sequence different from native IL-7 receptor because of a deletion, insertion, or substitution.

Bioequivalent analogs of IL-7 receptor proteins may be constructed by, for example, making various substitutions of residues or sequences or deleting terminal or internal residues or sequences not needed for biological activity. For example, cysteine residues can be deleted or replaced with other amino acids to prevent formation of incorrect intramolecular disulfide bridges upon renaturation. In addition, differences in the several cDNA clones isolated from various cell lines indicate that amino acid 46 (relative to cDNA clone H20) may be Ile or Thr, amino acid 118 may be Val or Ile, amino acid 224 may be Thr or Ile and amino acid 336 may be Ile or Val. Other approaches to mutagenesis involve modification of adjacent dibasic amino acid residues to enhance expression in yeast systems in which KEX2 protease activity is present. Generally, substitutions should be made conservatively; i.e., the most preferred substitute amino acids are those having physicochemical characteristics resembling those of the residue to be replaced. Similarly, when a deletion or insertion strategy is adopted, the potential effect of the deletion or insertion on biological activity should be considered.

Subunits of IL-7R, IL-2R, and IL-15R may be constructed by deleting terminal or internal residues or sequences. Subunits include those in which the transmembrane region and intracellular domain of IL-7R, IL-2R, or IL-15R or subunits thereof (α, β, or γc) are maintained and the extracellular domain(s) deleted or replaced.

Mutations in nucleotide sequences constructed for expression of analog IL-7R, IL-2R, and IL-15R must, of course, preserve the reading frame phase of the coding sequences and preferably will not create complementary regions that could hybridize to produce secondary mRNA structures such as loops or hairpins which would adversely affect translation of the receptor mRNA. Although a mutation site may be predetermined, it is not necessary that the nature of the mutation per se be predetermined. For example, in order to select for optimum characteristics of mutants at a given site, random mutagenesis may be conducted at the target codon and the expressed IL-7R mutants screened for the desired activity.

Not all mutations in the nucleotide sequence which encodes IL-7R will be expressed in the final product, for example, nucleotide substitutions may be made to enhance expression, primarily to avoid secondary structure loops in the transcribed mRNA (see EPA 75,444A, incorporated herein by reference), or to provide codons that are more readily translated by the selected host, e.g., the well-known E. coli preference codons for expression in E. coli.

Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene having particular codons altered according to the substitution, deletion, or insertion required. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462 disclose suitable techniques, and are incorporated by reference herein.

Expression of Nucleic Acids Encoding Modified IL-7R

In some embodiments, the present invention provides recombinant expression constructs or vectors, which include synthetic or cDNA-derived DNA fragments, encoding an IL-7R or bioequivalent analog operably linked to suitable transcriptional or translational regulatory elements derived from mammalian, microbial, viral, or insect genes and functional in an intended host or recipient cell in which the expression construct is to be expressed. Thus, a person of ordinary skill in the art can select regulatory elements for use in, for example, bacterial host cells, yeast host cells, mammalian host cells, and/or human host cells. Such regulatory elements may include a transcriptional promoter, an operator sequence to control transcription, a sequence encoding suitable mRNA ribosomal binding sites, and sequences which control transcription (e.g., transcription termination sequences) and translation (e.g., translation termination sequences), as described in detail below. Additional elements may be incorporated as appropriate for optimizing expression of the IL-7R, such as sequences encoding signal peptides, enhancers, and/or sequences that direct polyadenylation of mRNA. The ability to replicate in a host, usually conferred by an origin of replication, and a selection gene to facilitate recognition of transformants may additionally be incorporated.

Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the disclosure. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent.

In some embodiments, elements of an expression construct or vector may be operably linked, such as a promoter sequence operably linked to a polynucleotide sequence encoding a modified IL-7R as described herein. For example, DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Generally, operably linked means contiguous and, in the case of secretory leaders, contiguous and in reading frame.

DNA sequences encoding mammalian IL-7 receptors which are to be expressed in a microorganism will preferably contain no introns that could prematurely terminate transcription of DNA into mRNA; however, premature termination of transcription may be desirable, for example, where it would result in mutants having advantageous C-terminal truncations, for example, deletion of a transmembrane region to yield a soluble receptor not bound to the cell membrane. Due to the degeneracy of the genetic code, there can be considerable variation in nucleotide sequences encoding the same amino acid sequence, i.e., a variety of different polynucleotide sequences can encode polypeptides of the present disclosure. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides of the subject disclosure. These variant or alternative polynucleotide sequences are within the scope of the subject disclosure. As used herein, references to “essentially the same” sequence refers to sequences which encode amino acid substitutions, deletions, additions, or insertions which do not materially alter the functional activity of the polypeptide encoded by the polynucleotides of the present disclosure.

Polynucleotides encoding a polypeptide having one or more amino acid substitutions in the sequence are contemplated within the scope of the present disclosure. Amino acids can be generally categorized in the following classes: non-polar, uncharged polar, basic, and acidic. Conservative substitutions whereby a polypeptide of the present disclosure having an amino acid of one class is replaced with another amino acid of the same class fall within the scope of the subject disclosure so long as the polypeptide having the substitution still retains substantially the same functional activity as the polypeptide that does not have the substitution.

Exemplary DNA embodiments may include sequences capable of hybridizing to a sequence as described herein under moderately stringent conditions (50° C., 2×SSC) and other sequences hybridizing or degenerate to those described above, which encode biologically active IL-7R polypeptides.

Polynucleotides of the present disclosure can be composed of either RNA or DNA. Preferably, the polynucleotides are composed of DNA. The disclosure also encompasses those polynucleotides that are complementary in sequence to the polynucleotides disclosed herein. Polynucleotides and polypeptides of the disclosure can be provided in purified or isolated form.

Transformed host cells are cells which have been transformed or transfected with IL-7R, IL-2R, or IL-15R vectors constructed using recombinant DNA techniques. Expressed IL-7R will be deposited in the cell membrane or secreted into the culture supernatant, depending on the IL-7R, IL-2R, or IL-15R DNA selected. Suitable host cells for expression of mammalian IL-7R, IL-2R, or IL-15R include prokaryotes, yeast or higher eukaryotic cells under the control of appropriate promoters. Prokaryotes include gram negative or Gram-positive organisms, for example E. coli or bacilli. Higher eukaryotic cells include established cell lines of mammalian origin, including T cells, iNKT cells, and NK cells and cells of any of these types co-expressing chimeric antigen receptors. Cell-free translation systems could also be employed to produce mammalian IL-7R, IL-2R, and IL-15R using RNAs derived from the DNA constructs of the present invention. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference.

Prokaryotic expression hosts may be used for expression of IL-7Rs, IL-2Rs, and IL-15Rs that do not require extensive proteolytic and disulfide processing. Prokaryotic expression vectors generally comprise one or more phenotypic selectable markers, for example a gene encoding proteins conferring antibiotic resistance or supplying an autotrophic requirement, and an origin of replication recognized by the host to ensure amplification within the host. Suitable prokaryotic hosts for transformation include E. coli, Bacillus subtilis, Salmonella typhimurium, and various species within the genera Pseudomonas, Streptomyces, and Staphyolococcus, although any others appropriate in accordance with the disclosure may also be employed as a matter of choice.

Useful expression vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and pGEM1 (Promega Biotec, Madison, Wis., USA). These pBR322 “backbone” sections are combined with an appropriate promoter and the structural sequence to be expressed. E. coli is typically transformed using derivatives of pBR322, a plasmid derived from an E. coli species (Bolivar et al., Gene 2:95, 1977). pBR322 contains genes for ampicillin and tetracycline resistance and thus provides simple means for identifying transformed cells.

Promoters can be identified, characterized, and incorporated into a polynucleotide using standard techniques known in the art. Methods for identifying and characterizing promoter regions in genomic DNA are known in the art. Multiple copies of promoters or multiple promoters may be used in an expression construct or vector of the disclosure. In a preferred embodiment, a promoter may be positioned about the same distance from the transcription start site in the expression construct as it is from the transcription start site in its natural genetic environment. Some variation in this distance is permitted without substantial decrease in promoter activity. A transcription start site is typically included in the expression construct.

Promoters for expression of a desired gene in a host cell are known and available in the art. Constitutive promoters (e.g., ubiquitin or actin promoter), developmentally-regulated promoters, and inducible promoters (such as those promoters than can be induced by heat, light, hormones, or chemicals) are also contemplated for use with polynucleotide expression constructs of the disclosure. For example, promoters commonly used in recombinant microbial expression vectors include the β-lactamase (penicillinase) and lactose promoter system (Chang et al., Nature 275:615, 1978; and Goeddel et al., Nature 281:544, 1979), the tryptophan (trp) promoter system (Goeddel et al. Nucl. Acids Res. 8:4057, 1980; and EPA 36,776) and tac promoter (Maniatis, Molecule Cloning: A Laboratory Manual, Cold Spring Harbor Laboratory, p. 412, 1982). A particularly useful bacterial expression system employs the phage λPL promoter and cI857ts thermolabile repressor. Plasmid vectors available from the American Type Culture Collection which incorporate derivatives of the λPL promoter include plasmid pHUB2, resident in E. coli strain JMB9 (ATCC 37092) and pPLc28, resident in E. coli RR1 (ATCC 53082).

In some specific embodiments, a chimeric IL-7 receptor may be used as described herein. For example, in some embodiments, modifications may be made to individual components or regions or moieties of an IL-7, IL-15, or IL-2 receptor. Such components may include, for example, the intracellular domain (ICD), transmembrane domain (TMD), or the intracellular domain of an IL-7, IL-15, or IL-2 receptor. In some embodiments, either chain of a receptor as described herein may be modified as described, such as the IL-7 alpha chain (IL-7Rα), which may contain the components required for signaling related to CAR-T and T cell homeostasis and activation. Regions of these receptors may be hybridized or interchanged with, for example, an extracellular domain (ECD) of an unrelated protein linked by a CD8 linker domain. Expression of a resulting chimeric protein may allow for precise control of IL-7 signaling in T cells, such as therapeutic CAR-T cells, thus allowing for increased CAR-T activation and persistence independent of endogenous IL-7 cytokine production. Unlike previous attempts to bolster CAR-T activity using mutant IL-7 receptors, which are constitutively active (“always on”) (Zenatti et al., Nature Genetics 2011), the present approach allows for a calibrated approach, and therefore decreases the expected toxicity inherent to a constitutively active IL-7 receptor. The current approach allows titration in of an independent ligand in the form of a drug, such as an FDA-approved biologic drug, soluble protein, or other macromolecule, to activate IL-7 signaling in a target cell. This has the advantage of decreased toxicity, clinically controllable activation schemes, and the ability to vary the input ligand corresponding to an interchangeable ECD.

A DNA construct for a chimeric IL-7 receptor useful for the present disclosure may be expressed in a plasmid or other expression vector. For example, in some embodiments, a lentiviral plasmid vector such as pLVM-EF1a (Addgene) may allow for the generation of lentiviruses capable of transducing therapeutic CAR-T cells with the construct. A number of useful constructs are described in detail in the Examples. For example, a Human IgE CE3 IL-7Ra chimera referred to herein as “Construct 1” or “WU44” may comprise one or more components such as:

Domain 1: Kozak and CD8a signal peptide, set forth herein as SEQ ID NO:1;

Domain 2: Human IgE CE3 ECD, set forth herein as SEQ ID NO:2;

Domain 3: CD8 Linker ECD, set forth herein as SEQ ID NO:3;

Domain 4: IL-7Ra TMD, set forth herein as SEQ ID NO:4;

Domain 5: IL-7Ra ICD, set forth herein as SEQ ID NO:5; and/or

Domain 6: P2A-Thy1.1 co-expressed surface marker, set forth herein as SEQ ID NO:6;

In some embodiments, an Anti-NP clone 3B44 SCFV IL-7Ra chimera referred to herein as “Construct 2” or “WU45” may comprise one or more components such as:

Domain 1: Kozak and CD8a signal peptide (see construct 1);

Domain 2: Anti-NP 3B44 SCFV ECD, set forth herein as SEQ ID NO:7;

Domain 3: CD8 Linker ECD (see construct 1);

Domain 4: IL-7Ra TMD (see construct 1);

Domain 5: IL-7Ra ICD (see construct 1); and/or

Domain 6: P2A-Thy1.1 co-expressed surface marker (see construct 1).

In some embodiments, a Human Carcinoembryonioc Antigen (CEA) IL-7Ra chimera referred to herein as “Construct 3” or “WU46” may comprise one or more components such as:

Domain 1: Kozak and CD8a signal peptide (see construct 1);

Domain 2: CEA a2-b3 ECD, set forth herein as SEQ ID NO:8;

Domain 3: CD8 Linker ECD (see construct 1);

Domain 4: IL-7Ra TMD (see construct 1);

Domain 5: IL-7Ra ICD (see construct 1);

Domain 6: P2A-Thy1.1 co-expressed surface marker (see construct 1);

In some embodiments, a construct useful in accordance with the present disclosure, for example WU44, WU45, WU46, or other constructs encompassed within the scope of the present disclosure, may contain alternate domains as deemed appropriate. In a non-limiting example, WU44 may contain a different Domain 2 to include both the CE3 and CE4 domains, or CE2 through CE4 domains, or other domains as appropriate, without deviating from the present disclosure, etc. In other embodiments, WU44 may contain a different Domain 3, e.g., to have an Ig hinge-based linker, rather than a CD8 linker. One of skill in the art will understand and be able to identify alternate domains to a construct as described herein in order to achieve optimum results.

Ligands to Activate Chimeric IL-7 Receptor

In some embodiments, a ligand (e.g., a drug or soluble protein as described herein) may bind to and thereby activate a chimeric IL-7 receptor as described herein. For example, for Construct 1 (IgE_CE3 IL-7Ra chimera, “WU44”), Xolair® (omalizumab), an FDA approved biologic for the treatment of severe allergies or asthma, may bind to the C-epsilon 3 (CE3) domain of the human IgE antibody. Xolair® is an IgG1 monoclonal antibody with bivalent F_{ab} regions, allowing for the homodimerization of WU44 and downstream IL-7 receptor signal propagation.

In other embodiments, for Construct 2 (anti-NP 3B44 IL-7Ra chimera, “WU45”), 4-Hydroxy-3-nitrophenyl (NP) hapten may be conjugated to a large number of proteins at a high NP-to-protein ratio. In this case, NP may be conjugated to bovine serum albumin (BSA). The NP-decorated BSA binds to the anti-NP 3B44 SCFV ECD of WU45, causing the homodimerization of WU45 and downstream IL-7 receptor signal propagation.

In other embodiments, for Construct 3 (CEA IL-7Ra chimera, “WU46”), CEA-Scan (Arcitumomab) or CEA antigen, which is an FDA-approved murine IgG1 antibody for use in diagnostic imaging of colorectal cancers, and which is mainly expressed by human embryos, with limited expression in adult tissues, except in the case of certain colorectal cancers, binds to the CEA ECD of WU46 and allows for the homodimerization of WU46 and downstream IL-7 receptor signal propagation, similar to proposed binding of omalizumab to WU44.

Methods used for cloning and expression of a vector or construct as described herein are known in the art and described in the Examples.

Modification of Nucleic Acids Encoding a Modified IL-7R, IL-2R, or IL-15R

Any number of methods well known to those skilled in the art can be used to isolate and manipulate a nucleic acid molecule. In accordance with the disclosure, a nucleic acid may be DNA, RNA, or a DNA/RNA hybrid. Such nucleic acids may be modified using any appropriate methods known and/or available in the art. For example, PCR technology may be used to amplify a particular starting nucleic acid molecule and/or to produce variants of such a starting nucleic acid molecule. For example, as described herein, a nucleic acid encoding an IL-7R, IL-2R, or IL-15R or a fragment thereof, may be modified to bind or recognize a drug as described herein. Any modifications may be employed as deemed appropriate in accordance with the disclosure. Nucleic acid molecules, or fragments thereof, can also be obtained by any techniques known in the art, including directly synthesizing a fragment by chemical means. Thus, all or a portion of a nucleic acid as described herein may be synthesized. Likewise, all or any portion of a nucleic acid as described herein may be modified, e.g., modification of an IL-7R, IL-2R, or IL-15R to bind a drug.

As used herein, the term “complementary nucleic acids” refers to two nucleic acid molecules, e.g., DNA, RNA, cDNA, or hybrids thereof, that are capable of specifically hybridizing or binding to one another, wherein the two molecules are capable of forming an anti-parallel, double-stranded nucleic acid structure. Such hybridization or binding may be complete, such as two nucleic acid molecules that are 100% complementary, or may be incomplete, i.e., having one or more mismatches in sequence homology or complementarity. In some embodiments, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Stringency conditions are known in the art and would be understood by one of skill reading the present disclosure. One of skill in the art will also understand that stringency may be altered as appropriate to ensure optimum results. Complementarity as described herein also refers to the binding of a DNA editing enzyme to its target in vivo or in vitro. One of skill in the art would recognize that variations in complementarity will depend on the particular nucleic acid sequence and will be able to modify conditions as appropriate to account for this.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

Polynucleotides and polypeptides contemplated within the scope of the subject disclosure can also be defined in terms of more particular identity and/or similarity ranges with those sequences of the disclosure specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

Drugs and Soluble Proteins Useful for Binding to a Modified IL-7R, IL-2R, or IL-15R

In some embodiments, a modified IL-7R, IL-2R, or IL-15R as described herein binds a drug or soluble protein administered to a subject or patient, while not recognizing or binding to IL-7. Such a drug may be any drug for treatment of a disease, such as, for example, cancer. In some embodiments, a drug that may bind to a modified IL-7R, IL-2R, or IL-15R as described herein may include, but is not limited to, opioid antagonists (e.g., naltrexone, naloxone), vitamins (e.g., vitamin D, vitamin C, vitamin B6), cannabinoids (e.g., dronabinol), antibiotics, (e.g., fluoroquinolone antibiotics, e.g., floxacins such as trovafloxacin, temafloxacin, sparfloxacin, grepafloxacin, gatifloxacin, and alatrofloxacin; among others), Dihydrostreptomycin, coxibs (e.g., valdecoxib, rofecoxib, lumiracoxib), profens (e.g., suprofen, benoxaprofen), fenclozic acid, fenclofenac, NDRI antidepressants (e.g., nomifenisine), hydrazine MAOIs, such as tranylcypromine, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs (e.g., troglitazone), formins (e.g., phenformin, buformin), antihistamines (e.g., terfenadine), antihistamines (e.g., thenalidine), 5HT4 is [e.g., tegaserod maleate (Zelnorm)], Oxyphenisatine, nefazodone, levamisole (antihelminthic), Flosequinan (quinolone vasodilator), metamizole, dimethylamylamine (DMAA, Forthane).

In some embodiments, a soluble protein as used herein may be a recombinant protein or a fusion protein or an engineered protein. Such a recombinant protein may comprise a portion or fragment of a first protein and a portion or fragment of a second protein. In some embodiments, a recombinant protein may refer to a protein that is naturally occurring in an organism, or such a protein may be produced ex vivo and introduced into the organism. In some embodiments, such a recombinant protein may be introduced into an organism as a nucleic acid (i.e., DNA or RNA, or hybrids thereof) encoding the recombinant protein and produced as a result of transcription and/or translation. As described herein, a recombinant protein as described herein may be capable of binding to one or both of the IL-7 and IL-15 receptors. For example, in some embodiments, a recombinant protein may comprise all or a portion of an IL-7 protein, fused to all or a portion of an IL-15 protein and therefore be able to bind to both the IL-7 receptor and the IL-15 receptor.

In some embodiments, a drug or soluble protein that may bind to a modified IL-7R, IL-2R, or IL-15R receptor as described herein may include, but is not limited to:
an insulin or modified form or subunit thereof, including a human insulin or modified form thereof, preferably inactivated such that it does not bind to and/or activate the insulin receptor or otherwise modulate metabolic effects or act as a hormone, and preferably non-immunogenic (e.g., substantially human);
osteopontin (OPN) or a modified form or subunit thereof, including human osteopontin modified to resist degradation by extracellular enzymes. OPN may exist as different splice variants and proteolytic forms, e.g. OPN-a, OPN-b, OPN-c, osteopontin-L, osteopontin-R, and osteopontin-166. See, e.g., WO2018111852, published Jun. 21, 2018.

In other embodiments, a drug or soluble protein useful for use with the present disclosure may include a monoclonal antibody, such as including, but not limited to, Adalimumab (Amjevita®), Bezlotoxumab (Zinplava™), Avelumab (Bavencio®), Dupilumab (Dupixent®), Durvalumab (Imfinzi®), Ocrelizumab (Ocrevus™), Brodalumab (Siliq), Reslizumab (Cinqair™), Olaratumab (Lartruvo), Daratumumab (Darzalex®), Elotuzumab (Empliciti), Necitumumab (Portrazza), Infliximab (Inflectra), Obiltoxaximab (Anthim®), Atezolizumab (Tecentriq®), Secukinumab (Cosentyx™), Mepolizumab (Nucala), Nivolumab (Opdivo), Alirocumab (Praluent), Idarucizumab (Praxbind®), Evolocumab (Repatha®), Dinutuximab (Unituxin), Bevacizumab (Blincyto®), Pembrolizumab (Keytruda®), Ramucirumab (Cyramza), Vedolizumab (Entyvio®), Siltuximab (Sylvant®), Alemtuzumab (Lemtrada®), Trastuzumab emtansine (Kadcyla®), Pertuzumab (Perjeta®), Infliximab (Remsima®), Obinutuzumab (Gazyvaro®), Brentuximab (Adcetris®), Raxibacumab (ABthrax®), Belimumab (Benlysta®), Ipilimumab (Vervoy®), Denosumab (Xgeva®), Denosumab (Prolia®), Ofatumumab (Arzerra®), Besilesomab (Scintimun®), Tocilizumab (RoActemra®), Canakinumab (Ilaris®), Golimumab (Simponi®), Ustekinumab (Stelara®), Certolizumab pegol (Cimzia®), Catumaxomab (Removab®), Eculizumab (Soliris®), Ranibizumab (Lucentis®), Panitumumab (Vectibix®), Natalizumab (Tysabri®), Catumaxomab (Proxinium®), Bevacizumab (Avastin®), Omalizumab (Xolair®), Cetuximab (Erbitux®), Efalizumab (Raptiva®), Ibritumomab tiuxetan (Zevalin®), Fanolesomab (NeutroSpec®), Adalimumab (Humira®), Tositumomab and iodine 131 tositumomab (Bexxar®), Alemtuzumab (Campath®), Trastuzumab (Herceptin®), Gemtuzumab ozogamicin (Mylotarg®), Infliximab (Remicade®), Palivizumab (Synagis®), Necitumumab (Daclizumab), Basiliximab (Simulect®), Rituximab (Rituxan® MabThera®), Votumumab (Humaspect®), Sulesomab (LeukoScan®), Arcitumomab (CEA-scan®), Imiciromab (MyoScint®), Capromab (ProstaScint®), Nofetumomab (Verluma®), Abciximab (ReoPro®), Satumomab (OncoScint®), and/or Muromonab-CD3 (Orthoclone OKT3®). See, e.g., ACTIP's list of monoclonal antibodies approved by the EMA and FDA for therapeutic use.

In some embodiments, a drug useful for binding to a chimeric receptor of the present disclosure may be omalizumab, i.e., Xolair®. Omalizumab is a recombinant DNA-derived humanized IgG1κ monoclonal antibody that selectively binds to human immunoglobulin E (IgE), which helps to block its action and thus decrease allergic responses in the body. Unlike an ordinary anti-IgE antibody, omalizumab does not bind to IgE that is already bound by the high affinity IgE receptor (FcεRI) on the surface of mast cells, basophils, and antigen-presenting dendritic cells. The antibody has a molecular weight of approximately 149 kD. In some embodiments, omalizumab is produced by a Chinese hamster ovary (CHO) cell suspension culture in a nutrient medium containing an antibiotic, for example including, but not limited to, gentamicin.

Discussion of anti-IgE therapeutic antibodies and their pharmacological mechanisms can be found at least in Chang et al., Adv Immunol Advances in Immunology 93:63-119, 2007; Chang, Nat Biotechnol 18(2):157-62, 2000; and Chang et al., J Allergy and Clinical Immunology 117(6):1203-12, 2006). Omalizumab inhibits the binding of IgE to FcεRI on mast cells and basophils by binding to an antigenic epitope on IgE that overlaps with the site to which FcεRI binds. This feature is critical to its pharmacological effects because a typical anti-IgE antibody can cross-link cell surface FcεRI-bound IgE, thereby aggregate FcεRI, and activate mast cells and basophils to discharge the horde of chemical mediators stored in the densely packed sacs inside the cells. However, when IgE is bound to the receptor, the antigenic epitope on IgE to which omalizumab binds is sterically hindered by the receptor and is not accessible to omalizumab binding, thus averting the anaphylactic effects presumably unavoidable with an ordinary anti-IgE antibody. Furthermore, although the peptide elements on IgE involved in binding to low affinity IgE receptor (FcεRII) on many cell types are different from the peptide elements involved in binding to FcεRI, omalizumab, by steric hindrance, also prevents binding of IgE to FcεRII. The reduced binding of IgE to both FcεRI and FcεRII has profound effects on the attenuation of IgE-mediated allergic responses.

Free IgE in patients is depleted by omalizumab, the FcεRI receptors on basophils, mast cells, and dendritic cells are gradually down-regulated with somewhat different kinetics, rendering those cells much less sensitive to the stimulation by allergens. Thus, in this regard, therapeutic anti-IgE antibodies represent a class of potent mast cell stabilizers, providing the fundamental mechanism for the effects of omalizumab on various allergic and non-allergic diseases involving mast cell degranulation. Many investigators have identified or elucidated a host of pharmacological effects, which help bring down the inflammatory status in the omalizumab-treated patients.

In some embodiments, omalizumab may be administered to a patient by any mode, including, but not limited to, subcutaneous injection, intravenous injection, and intravenous infusion.

Each of the above drugs/soluble proteins has multiple epitopes which may be targeted by scFvs which replace the IL-7, IL-2- or IL-15 binding domains on one or more of the subunits of those proteins (or may provide epitopes by way of combination with or conjugation to a hapten).

The drug or soluble protein may have one or more of the following features:
Has a high therapeutic index at doses intended for stimulation of proliferation of CAR-bearing immune effector cells which have the modified IL-7R, Il-15R, or IL-2R disclosed herein in a patient;
Does not cause off-target effects when administered to a patient (e.g., does not bind, or does not activate, other effector proteins, at least to a degree that causes dose-limiting side effects);
Does not have analogues which, if these analogues were ingested or administered to the patient, could inadvertently activate the receptor to a degree that would cause undesired effects;
Is an approved drug in the jurisdiction where approval of the CAR-bearing immune effector cells which have the modified IL-7R, Il-15R, or IL-2R disclosed herein is sought;
Is off-patent in the jurisdiction where approval of the CAR-bearing immune effector cells which have the modified IL-7R, Il-15R, or IL-2R disclosed herein is sought; and
Can be administered IV and/or orally.

Genome-Edited CAR-Bearing Immune Effector Cells

CAR-bearing immune effector cells, such as CAR-T cells, CAR-iNKT cells, and CAR-NK cells encompassed by the present disclosure are deficient in one or more antigens to which the chimeric antigen receptor specifically binds and are therefore fratricide-resistant. In some embodiments, the one or more antigens of the T cell is modified such the chimeric antigen receptor no longer specifically binds the one or more modified antigens. For example, the epitope of the one or more antigens recognized by the chimeric antigen receptor may be modified by one or more amino acid changes (e.g., substitutions or deletions) or the epitope may be deleted from the antigen. In other embodiments, expression of the one or more antigens is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T cell is modified such that the one or more antigens is not expressed, e.g., by deletion or disruption of the gene encoding the one or more antigens. In each of the above embodiments, the CAR-bearing immune effector cell may be deficient in one or preferably all the antigens to which the chimeric antigen receptor specifically binds. Methods for genetically modifying a T cells, iNKT cells, and NK cells to be deficient in one or more antigens are well known in art. In an exemplary embodiment, CRISPR/cas9 gene editing can be used to modify a T cell, iNKT cell, or NK cell to be deficient in one or more antigens.

CAR-bearing immune effector cells, such as CAR-T cells, encompassed by the present disclosure may further be deficient in endogenous T cell receptor (TCR) signaling as a result of deleting a part of the T Cell Receptor (TCR)-CD3 complex. In various embodiments it may be desirable to eliminate or suppress endogenous TCR signaling in CAR-T cells disclosed herein. For example, decreasing or eliminating endogenous TCR signaling in CAR-T cells may prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3.epsilon, CD3.gamma, CD3.delta, and/or CD3.gamma. Deleting a part of the TCR receptor complex may block TCR mediated signaling and may thus permit the safe use of allogeneic T cells as the source of CAR-T cells without inducing life-threatening GvHD.

Alternatively, or in addition, CAR-bearing immune effector cells encompassed by the present disclosure may further comprise one or more suicide genes. As used herein, “suicide gene” refers to a nucleic acid sequence introduced to a such a cell by standard methods known in the art that, when activated, results in the death of the CAR-bearing immune effector cell e.g. CAR-T cell. Suicide genes may facilitate effective tracking and elimination of the CAR-bearing immune effector cells, e.g., CAR-T cells, in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

Dual CAR-T Cells

A genome-edited, tandem CAR-T cell, i.e., CD2*CD3e-dCARTΔCD2ΔCD3ε, may be generated by cloning a commercially synthesized anti-CD2 single chain variable fragment into a lentiviral vector containing a 3rd generation CAR backbone with CD28 and 4-1BB internal signaling domains and cloning a commercially synthesized anti-CD3e single chain variable into the same lentiviral vector containing an additional 3rd generation CAR backbone with CD28 and 4-1BB internal signaling domains resulting in a plasmid from which the two CAR constructs are expressed from the same vector. See Example 6, Table 1.

Tandem CAR-T Cells

A tandem CAR-T cell (equivalently, tCAR-T) is a T cell with a single chimeric antigen polypeptide containing two distinct antigen recognition domains with affinity to different targets wherein the antigen recognition domains are linked through a peptide linker and share common costimulatory domain(s), wherein binding of either antigen recognition domain will signal though a common co-stimulatory domains(s) and signaling domain. See Example 6, Table 1.

Mono iNKT-CAR Cells

In certain embodiments, the disclosure provides an engineered iNKT cell comprising a single CAR, that specifically binds CD7, wherein the iNKT cell is deficient in CD7 (e.g., CD7-iNKT-CARΔCD7 cell). In non-limiting examples, the deficiency in CD7 resulted from (a) modification of CD7 expressed by the iNKT cell such that the chimeric antigen receptors no longer specifically binds the modified CD7, (b) modification of the iNKT cell such that expression of CD7 is reduced in the iNKT cell by at least 50%, at least 60%, at least 70%, at least 80%, at least 90% or more, or (c) modification of the iNKT cell such that CD7 is not expressed (e.g., by deletion or disruption of the gene encoding CD7. In further embodiments, the iNKT cell comprises a suicide gene. In non-limiting examples the suicide gene expressed in the CD7-iNKT-CARΔCD7 cells encodes a modified Human-Herpes Simplex Virus-1-thymidine kinase (TK) gene fused in-frame to the extracellular and transmembrane domains of the human CD34 cDNA. The CAR for a CD7 specific iNKT-CAR cell may be generated by cloning a commercially synthesized anti-CD7 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making CARs specific for other malignant T cell antigens.

In a similar manner, other mono iNKT-CARs may be constructed, and are given below in Table 2 of Example 7.

Tandem iNKT-CAR Cells

In certain embodiments, the disclosure provides an engineered iNKT cell comprising a tandem CAR (tCAR), i.e., two scFv sharing a single intracellular domain, that specifically binds CD7 and CD2, wherein the iNKT cell is deficient in CD7 and CD2 (e.g., CD7×CD2-iNKT-tCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-tCARΔCD7ΔCD2 cells encodes a modified Human-Herpes Simplex Virus-1-thymidine kinase (TK) gene fused in-frame to the extracellular and transmembrane domains of the human CD34 cDNA.

A tCAR for a genome-edited, tandem iNKT-CAR cell, i.e., CD7*CD2-iNKT-tCARΔCD7ΔCD2, may be generated by cloning a commercially synthesized anti-CD7 single chain variable fragment (scFv) and an anti-CD2 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making tCARs specific for other malignant T cell antigens.

In a similar manner, other tandem iNKT-CARs may be constructed, and are given below in Tables 3-4 of Example 7.

Dual iNKT-CAR Cells

In certain embodiments, the disclosure provides an engineered iNKT cell comprising a dual CAR (dCAR), i.e., two CARs expressed from a single lentivirus construct, that specifically binds CD7 and CD2, wherein the iNKT cell is deficient in CD7 and CD2 (e.g., CD7×CD2-iNKT-dCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-dCARΔCD7ΔCD2 cells encodes a modified Human-Herpes Simplex Virus-1-thymidine kinase (TK) gene fused in-frame to the extracellular and transmembrane domains of the human CD34 cDNA. In a similar manner, other dual iNKT-CARs may be constructed, and are given below in Tables 3-4 of Example 7.

Mono, Dual, and Tandem NK-CAR Cells

As disclosed above, CAR-bearing natural killer cells (CAR-NKs or NK-CARs) can be made.

### Antigen Targets of CARs

Antigens which the CARs, to be used in the CAR-bearing immune effector cells comprising the chimeric IL-7, IL-15, or IL-2 receptors disclosed herein, may bind, include but are not limited to antigens present on the surface of lymphomas and leukemias including B-cell cancers, T-cell cancers, and multiple myeloma.

In certain embodiments, the antigen(s) is/are chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

In certain embodiments, the chimeric antigen receptor(s) specifically binds at least one antigen expressed on a malignant T cell. In certain embodiments, the antigen expressed on a malignant T cell is chosen from CD2, CD3, CD4, CD5, CD7, TCRA, and TCRβ.

In certain embodiments, the chimeric antigen receptor specifically binds at least one antigen expressed on a malignant plasma cell In certain embodiments, the antigen expressed on a malignant plasma cell is chosen from BCMA, CS1, CD38, CD79A, CD79B, CD138, and CD19.

In certain embodiments, the chimeric antigen receptor is constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma.

In certain embodiments, the chimeric antigen receptor(s) specifically binds at least one antigen expressed on a malignant B cell. In certain embodiments, the antigen expressed on a malignant B cell is chosen from CD19, CD20, CD21, CD22, CD23, CD24, CD25, CD27, CD38, and CD45.

In certain embodiments, the antigen expressed on a malignant B cell is chosen from CD19 and CD20.

Generation of scFv Antibodies

scFv (single chain fragment variable) antibodies have been constructed mainly from hybridoma (Galeffi et al., 2006). scFv (single chain fragment variable) is a noncovalent heterodimer comprised of the VH and VL domains which can then be used in the construction of recombinant scFv (single chain fragment variable) antibody. In order to attain them, mRNA is first isolated from hybridoma followed by reverse transcribed into cDNA to serve as a template for antibody genes amplification (PCR). With this method, large libraries with a diverse range of antibodies and genes can be created.

In the scFv (single-chain fragment variable) construction, the order of the domains can be either -linker- or -linker- and both orientations have been applied One of the most popular methods used in construction is through PCR assembly. This method allows the domains of antibody to be cloned without any prior information about the nucleic acid as well as amino acid sequence of the particular antibody. Moreover, the domains of antibody can be combined by in vitro recombination directly after the PCR of and genes into plasmid or phagemid. Alternatively, scFv (single-chain fragment variable) can also be constructed with sequential cloning or combinatorial infection.

To date, antibody fragments (scFv) have been successfully isolated and displayed as fragments in various expression systems such as mammalian cell and yeast, plant, and also insect cells. scFv (single-chain fragment variable) antibody can be expressed as correctly folded and directly active proteins or as aggregates requiring in vitro refolding to become active. Depending on the expression system, it varies in their ability to fold and secrete the scFv (single-chain fragment variable) proteins. There are some general regulations to consider on the design of vectors and expression system used with the different hosts and each of this host has advantages and disadvantages for the production of active scFv (single-chain fragment variable) antibody. Nevertheless, the bacterial expression system is most often applied for the production of scFv (single-chain fragment variable) antibody fragments compared to the various expression strategies available.

Recent progress in the perceptive of both genetics and biochemistry of E. coli makes this organism a precious tool as an expression host. Moreover, an scFv (single-chain fragment variable) antibody with good folding properties can be expressed economically by using this organism compared to whole antibodies with complicated folding and glycosylation which require slow and expensive cell culture techniques. Furthermore, this production system is well-studied physiology, genetics, and availability of advanced genetic tools, rapid growth, very high yields up to 10-30% of total cellular protein, a simple way to conduct within a standard molecular biology laboratory, low cost, and the capability to multiplex both expression screening together with the protein production.

ScFvs for use in the modified IL-7Rs, IL-2Rs, and IL-15Rs disclosed herein may be generated by the methods above and by methods known in the art.

The Expression of Antibody Fragments (scFv)

There are a number of different strategies used to express the recombinant antibody fragments in E. coli. One way is to express scFv (single-chain fragment variable) antibody directly in the cytoplasm of E. coli without using a signal peptide. As a result, the polypeptides are greatly expressed in the reducing environment of bacterial cytoplasm followed by the formation of insoluble aggregates called inclusion bodies. For that reason, the inclusion bodies must be renatured in vitro to improve the correct folding of functional protein by means of appropriate rearrangement of the disulfide bonds. To overcome this problem, a signal peptide is used to direct secretion of the scFv (single-chain fragment variable) antibody into the periplasmic space that lies between inner and outer membranes of gram-negative bacteria. This periplasmic space is identified to contain protein such as chaperones and disulfide isomerases, which assist the proper folding of recombinant proteins. Generally, the Fab fragment produced in E. coli is based on the expression of a di-cistronic operon unit where both of the genes are controlled by the same promoter and thus allowing synthesis of both chains in an equal amount. During translocation via the inner membrane into the oxidizing environment of the periplasm, the signal peptides attached at the N-terminus are sliced off permitting the chains to fold and assemble and thus lead to the formation of both of the intra- and interdomain sulfide. Until now, periplasmic expression has been extensively employed in the production of various specific scFv (single-chain fragment variable) antibodies. But, in some cases, antibodies are observed as insoluble material since their efficiency and folding are depending on the individual protein.

Cell Transformation or Transfection

Methods of transformation of cells are well-known in the art. Techniques for transforming cells with a gene include, for example, biolistic methods, electroporation, DNA coated particles, calcium chloride treatment, PEG-mediated transformation, etc. (see, e.g., Nagel et al., 1990; Song et al., 2006; de la Pena et al., 1987; and Klein et al., 1993). Suitable methods for transformation of host cells for use with the current disclosure are believed to include virtually any method by which DNA can be introduced into a cell (see, e.g., Miki et al., 1993), for example by acceleration of DNA coated particles (U.S. Pat. Nos. 5,015,580; 5,550,318; 5,538,880; 6,160,208; 6,399,861; and 6,403,865), use of viral vectors, etc. For example, in some embodiments, an IL-7R as described herein may be included in the same vector as a CAR, expressed from a different promotor or as a polycistron separated by a 2A element or an IRES element. Through the application of techniques such as these, the cells of virtually any species may be stably transformed.

Following transformation, cells (e.g., CAR-T cells) may be selected and grown in culture. Cultured cells can be evaluated to identify cells that express a polynucleotide of the disclosure using standard methods known in the art. The transformed and cultured cells containing and/or expressing a modified IL-7R as described herein are also included within the scope of the present disclosure.

Various methods for selecting transformed cells have been described. For example, one might utilize a drug resistance marker to confer resistance to, e.g., an antibiotic. These exemplary approaches can each be used effectively to isolate a cell or multicellular organism or tissue thereof that has been transformed and/or modified as described herein.

In some embodiments, a non-naturally occurring sequence-specific or sequence-directed exogenous nucleic acid may be introduced into a cell (e.g., a T cell or CAR-T cell) in order to introduce a mutation in a specified gene in the cell, or in a plurality of cells obtained from a subject or patient as described herein. In some embodiments, a cell into which such a non-naturally nucleic acid may be cultured to obtain a population of cells. The ability to generate such a cell, or a plurality of cells derived therefrom depends on introducing an exogenous nucleic acid into the cell using, for example, transformation vectors and cassettes described herein.

A polypeptide useful in accordance with the disclosure may be isolated, non-naturally occurring, recombinant, or engineered nucleic acid-binding proteins that have nucleic acid or DNA binding regions containing polypeptide monomer repeats designed to target specific nucleic acid sequences.

The IL-7R, Il-2R, or IL-15R could be included in the same vector as the CAR for insertion into a CAR-bearing immune effector cell, expressed from a different promotor or as a polycitron separated by 2a or IRES element.

Detection of Exogenous Nucleic Acids in Recipient Cells

The disclosure also provides molecular assays for detecting and characterizing cells that have been modified as described herein. These assays include but are not limited to genotyping reactions, a PCR assay, a sequencing reaction or other molecular assay. Design and synthesis of nucleic acid primers useful for such assays, for instance to assay for the occurrence of a recombination event, are also contemplated. Genotyping of cells may be performed on any cells or tissue as appropriate with the disclosure, e.g., T cells or CAR-T cells. The genotype of such cells can be determined by, for example, PCR analysis, using PCR amplification of the IL-7R gene. In some embodiments, for a peptide having a protein tag, modified cells, i.e., produced through transfection, may also be detected using flow cytometry.

If the peptides have a protein tag, transfection can also be detected using flow cytometry.

### Kits

The disclosure further provides a kit comprising a composition comprising a population of CAR-bearing immune effector cells, e.g., CAR-T cells, as described herein. In some embodiments, such a kit may provide a composition in one or more single-use containers, for administration to a subject or patient. In other embodiments, sterile reagents and/or supplies for administration of a composition as described herein, may be provided as appropriate. In some embodiments, a kit of the disclosure may comprise a unit dosage of a composition as described herein for use in treatment of cancer. Such a unit dosage may be administered to a patient in a treatment setting as deemed appropriate by a clinician or practitioner.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, cell transformation/transfection, viral/cell culture, blood assays, e.g., complete blood count (CBC), viral titer/viral load assays, antibody assays, genetic complementation assays, or any assay useful in accordance with the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, substrates, antibodies, ligands, detection reagents, media components, such as salts including MgCl2, a polymerase enzyme, deoxyribonucleotides, ribonucleotides, and the like, reagents for DNA isolation, or the like, as described herein. Such reagents or components are well known in the art. Where appropriate, reagents included with such a kit may be provided either in the same container or media as a primer pair or multiple primer pairs, or may alternatively be placed in a second or additional distinct container into which an additional composition or reagents may be placed and suitably aliquoted. Alternatively, reagents may be provided in a single container means. A kit of the disclosure may also include packaging components, instructions for use, including storage requirements for individual components as appropriate. Such a kit as described herein may be formulated for use in a clinical setting, such as a hospital, treatment center, or clinical setting, or may be formulated for personal use as appropriate.

Indications and Standards of Care in ACT (e.g. CAR-T) Therapy

In some embodiments, the immune effector cells comprising the chimeric IL-7, IL-15, and IL-2 receptors disclosed herein express one or more chimeric antigen receptors (CARs) and can be used as a medicament, i.e., for the treatment of disease. In many embodiments, the cells are CAR-T cells. In other embodiments, the cells may be CAR-iNKT or CAT-NK cells.

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin's lymphoma, and T-cell chronic lymphocytic leukemia (T-CLL).

In some embodiments, the cells may be used in the treatment of autoimmune diseases such as lupus, autoimmune (rheumatoid) arthritis, multiple sclerosis, transplant rejection, Crohn's disease, ulcerative colitis, dermatitis, and the like. In some embodiments, the cells are chimeric autoantibody receptor T-cells, or CAR-Ts displaying antigens or fragments thereof, instead of antibody fragments; in this version of adoptive cell transfer, the B cells that cause autoimmune diseases will attempt to attack the engineered T cells, which will respond by killing them.

In some embodiments, the cells may be used in the treatment of infectious diseases such as HIV and tuberculosis.

In some embodiments, the treatment of a patient with CAR-T cells of the present disclosure can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic, is meant that the cells or population of cells used for treating patients are not originating from the patient but from a donor.

The treatment of cancer with CAR-T cells of the present disclosure may be in combination with one or more therapies selected from antibody therapy, chemotherapy, cytokine therapy, dendritic cell therapy, gene therapy, hormone therapy, radiotherapy, laser light therapy, and radiation therapy.

The administration of CAR-T cells or a population of CAR-T cells of the present disclosure of the present disclosure be carried out by aerosol inhalation, injection, ingestion, transfusion, implantation or transplantation. The CAR-T cells compositions described herein, i.e., mono CAR, dual CAR, tandem CARs, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

The administration of CAR-T cells or a population of CAR-T cells can consist of the administration of 10⁴-10⁹ cells per kg body weight, preferably 10⁵ to 10⁶ cells/kg body weight including all integer values of cell numbers within those ranges. The CAR-T cells or a population of CAR-T cells can be administrated in one or more doses. In another embodiment, the effective amount of CAR-T cells or a population of CAR-T cells are administrated as a single dose. In another embodiment, the effective amount of cells are administered as more than one dose over a period time. Timing of administration is within the judgment of a health care provider and depends on the clinical condition of the patient. The CAR-T cells or a population of CAR-T cells may be obtained from any source, such as a blood bank or a donor. While the needs of a patient vary, determination of optimal ranges of effective amounts of a given CAR-T cell population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of CAR-T cells or a population of CAR-T cells or composition comprising those CAR-T cells are administered parenterally. The administration can be an intravenous administration. The administration of CAR-T cells or a population of CAR-T cells or composition comprising those CAR-T cells can be directly done by injection within a tumor.

In one embodiment of the present disclosure, the CAR-T cells or a population of the CAR-T cells are administered to a patient in conjunction with, e.g., before, simultaneously or following, any number of relevant treatment modalities, including but not limited to, treatment with cytokines, or expression of cytokines from within the CAR-T, that enhance T-cell proliferation and persistence and, include but not limited to, IL-2, IL-7, and IL-15 or analogues thereof.

In some embodiments, the CAR-T cells or a population of CAR-T cells of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the CAR-T cells or a population of CAR-T cells of the present disclosure may be used in combination with T-cell checkpoint inhibitors, including but not limited to, anti-CTLA4 (Ipilimumab) anti-PD1 (Pembrolizumab, Nivolumab, Cemiplimab), anti-PDL1 (Atezolizumab, Avelumab, Durvalumab), anti-PDL2, anti-BTLA, anti-LAG3, anti-TIM3, anti-VISTA, anti-TIGIT, and anti-KIR.

In another embodiment, the CAR-T cells or a population of CAR-T cells of the present disclosure may be used in combination with T cell agonists, including but not limited to, antibodies that stimulate CD28, ICOS, OX-40, CD27, 4-1BB, CD137, GITR, and HVEM.

In another embodiment, the CAR-T cells or a population of CAR-T cells of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, the CAR-T cells or a population of CAR-T cells of the present disclosure may be used in combination with immunostimulatory therapies, such as toll-like receptors agonists, including but not limited to, TLR3, TLR4, TLR7 and TLR9 agonists.

In another embodiment, the CAR-T cells or a population of CAR-T cells of the present disclosure may be used in combination with stimulator of interferon gene (STING) agonists, such as cyclic GMP-AMP synthase (cGAS).

Immune effector cell aplasia, particularly T cell aplasia, is also a concern after adoptive cell transfer therapy. When the malignancy treated is a T-cell malignancy, and CAR-T cells target a T cell antigen, normal T cells and their precursors expressing the antigen will become depleted, and the immune system will be compromised. Accordingly, methods for managing these side effects are attendant to therapy. Such methods include selecting and retaining non-malignant T cells or precursors, either autologous or allogeneic (optionally engineered not to cause rejection or be rejected), for later expansion and re-infusion into the patient, after CAR-T cells are exhausted or deactivated. Alternatively, CAR-T cells which recognize and kill subsets of TCR-bearing cells, such as normal and malignant TRBC1⁺, but not TRBC2⁺ cells, or alternatively, TRBC2⁺, but not TRBC1⁺ cells, may be used to eradicate a T cell malignancy while preserving sufficient normal T cells to maintain normal immune system function.

### Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR § 1.822 is used.

Activate: As used herein, refers to making operative or functional, initiating, or initiating. For example, a binding of a drug to an ectodomain of a modified IL-7R as described herein activates, initiates, enables, sets in motion, etc., IL-7 signaling in a cell in which the modified IL-7R is present.

Allogeneic CAR-T cells are engineered using T cells from a single donor that are utilized in multiple patients.

Autologous CAR-T cells are harvested from a patient and culture expanded ex vivo to large quantities over many weeks and then returned to the patient.

Biologically active: When used to described an IL-7 receptor, means that a particular molecule shares sufficient amino acid sequence similarity with a modified IL-7 receptor of the present disclosure to be capable of binding detectable quantities of IL-7 or a drug, transmitting an IL-7 stimulus to a cell, for example, as a component of a hybrid receptor hybrid receptor construct, or cross-reacting with anti-IL-7R antibodies raised against IL-7R from natural (i.e., nonrecombinant) sources. Preferably, biologically active IL-7 receptors within the scope of the present invention are capable of binding greater than 0.1 nmoles IL-7 or drug per nmole receptor, and most preferably, greater than 0.5 nmole IL-7 or drug per nmole receptor in standard binding assays.

CAR-bearing immune effector cell(s): Refers to a T cell, an NK cell, an iNKT cell, a population of T cells, a population of NK cells, or a population of iNKT cells which comprise at least one or more chimeric antigen receptors (CARs), and includes chimeric antigen receptor (CAR)-bearing T cells (CAR-Ts), (CAR)-bearing invariant NKT (iNKT) cells (CAR-iNKTs), and CAR-bearing Natural Killer (NK) cells (CAR-NKs). A CAR is a receptor protein or fragment thereof that has been engineered to have the functions of antigen binding and T cell activation. CAR-T cells thus are able to target a specific protein. As described herein, CAR-T cells of the present disclosure express a modified IL-7R that binds a drug instead of the native IL-7 ligand.

Cluster of Differentiation (CD): A cell surface molecule recognized by antibodies. Expression of some CDs (e.g., CD4, CD8, CD25, CD127) is specific for cells of a particular lineage or maturational pathway, and the expression of others varies according to the state of activation, position, or differentiation of the same cells. Preferably, in some embodiments, the CD determinants are human when the isolated cells are to be administered to a human or a human immune response is being studied.

Disease, Condition, Disorder: As used herein, a disease, condition, disorder, and/or reaction or response to be treated according to the methods and compositions of the present disclosure refers to a disease or condition in which the immune system contributes to pathogenesis, for example T cells. These reactions may include, but are not limited to, cancer and cancerous conditions, autoimmune conditions, disorders, or diseases and persistent and progressive immune reactions to infectious non-self-antigens from bacterial, viral (e.g., HCV), fungal, or parasitic organisms which invade and persist within mammals and humans.

DNA sequence: A DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the sequence and its component nucleotide sequences by standard biochemical methods, for example, using a cloning vector. Such sequences are preferably provided in the form of an open reading frame uninterrupted by internal non-translated sequences, or introns, which are typically present in eukaryotic genes. In some embodiments, genomic DNA containing the relevant sequences could also be used. Sequences of non-translated DNA may be present 5′ or 3′ from the open reading frame, where the same do not interfere with manipulation or expression of the coding regions.

Drug: Refers to a non-IL-7 ligand capable of binding to a modified IL-7R as described herein. A drug may be any non-IL-7 ligand that can bind to such a modified IL-7R.

Drug recognition domain: As used herein, a drug recognition domain refers to a region on a modified IL-7 receptor that binds to a drug as a ligand.

Ectodomain: Equivalently, extracellular domain (ECD), refers to a portion, region, or domain of a protein or polypeptide on a cell membrane that extends beyond the plasma membrane into the extracellular space. An ectodomain may refer to such a portion or domain on a cell surface receptor protein. For example, as described herein, a modified IL-7R may have an ectodomain that may be a drug recognition domain and bind to a drug for activation of IL-7 signaling in a cell.

Endodomain: Equivalently, intracellular domain (ICD), refers to a portion, region, or domain of a protein or polypeptide that is internal to the cell, rather than extending into the extracellular space. As used herein, an endodomain may refer to a region on a transmembrane receptor protein that is in the cell interior and is involved in cellular signaling. An endodomain may also be referred to herein as an intracellular domain or portion of a protein. For example, some endodomains may provide, a protein kinase function to a cell for specific cellular signaling pathways, such as IL-7 signaling in a cell.

Enhanced: As used herein, refers to an elevation, stimulation, increase, intensification, magnification, improvement, etc., of, for example, binding of a ligand, transmission of a cellular signal, or the like. Enhanced may also be used to refer to an enhancement of immune function, such as an increased immune response or increased T cell function, T cell expansion, or the like. An enhanced immune response may refer to a favorable response in a subject or patient to treatment with, for example, CAR-T cells expressing a modified IL-7 receptor as described herein. In such a case, an enhanced immune response may mean increased activity of the immune system in elimination or control of cancer cells. Control may refer to killing of cancer cells, or inhibiting their growth as a result of treatment of a patient with CAR-T cells expressing a modified IL-7R as described herein.

Expression Construct: A combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation. An expression construct or vector may also be defined as a DNA construct that includes an encoded exogenous nucleic acid protein that can be transcribed. As used herein, a “recombinant expression vector” refers to a replicable DNA construct used either to amplify or to express DNA that encodes a modified IL-7 receptor as described herein, and which includes a transcriptional unit comprising an assembly of (1) a genetic element or elements having a regulatory role in gene expression, for example, promoters or enhancers, (2) a structural or coding sequence that is transcribed into mRNA and translated into protein, and (3) appropriate transcription and translation initiation and termination sequences. Structural elements intended for use in yeast expression systems preferably include a leader sequence enabling extracellular secretion of translated protein by a host cell. Alternatively, where recombinant protein is expressed without a leader or transport sequence, it may include an N-terminal methionine residue. This residue may optionally be subsequently cleaved from the expressed recombinant protein to provide a final product.

Exogenous DNA sequence: A DNA sequence that originates outside the host cell. Such a DNA sequence can be obtained from a different species, or the same species, as that of the cell into which it is being delivered.

Functionally similar: Refers to the activity of a modified IL-7R, IL-15R, or IL-2R of the disclosure that is equivalent or as effective in activity as a native IL-7/IL-15/IL-2 receptor. For example, a modified IL-7R, IL-15R, or IL-2R as disclosed herein may bind to a ligand such as a drug or soluble protein as described herein with a specificity and/or sensitivity equivalent to a native IL-7/IL-15/IL-2 receptor binding to a native IL-7/IL-15/IL-2 ligand. In some embodiments, functionally similar may refer to identical function or activity, for example a modified IL-7/IL-15/IL-2 receptor of the disclosure having identical activity, binding capacity, specificity, or the like, when compared to a native IL-7/IL-15/IL-2 receptor and its native IL-7/IL-15/IL-2 ligand. In some embodiments, a modified IL-7R as disclosed herein will also initiate downstream signaling. The modified IL-7Rs as disclosed herein typically retain an endodomain that is equivalent or sufficiently similar in sequence and/or structure to the native IL-7R endodomain.

Immune cell: Any cells of the immune system that may be assayed, including, but not limited to, B lymphocytes (B cells), T lymphocytes (T cells), natural killer (NK) cells, natural killer T (NK) cells, monocytes, macrophages, neutrophils, granulocytes, mast cells, platelets, Langerhans cells, stem cells, dendritic cells, peripheral blood mononuclear (PBMN) cells, tumor-infiltrating (TIL) cells, genetically modified immune cells, including hybridomas, drug modified immune cells, and derivatives, precursors or progenitors of the above cell types. In some specific embodiments, an immune cell of the disclosure refers to a T cell expressing a chimeric antigen receptor, also referred to herein as a CAR-T cell.

Genome-edited CAR-T cells: refers to a T cell or population of T cells which comprise at least one or more chimeric antigen receptors (CARs) targeting one or more antigens, wherein CAR-T cell is deficient in a subunit of the T cell receptor complex and at least one or more antigens to which the at least one or more CARs specifically binds. In some embodiments, the genome-edited CAR-T cell is deficient in the T cell receptor complex subunit selected from TCRA, TCRβ, CD3ε, CD3γ, CD3δ, and CD3ζ. In some additional embodiments, the genome-edited CAR-T cell, comprise at least one or more chimeric antigen receptors (CARs) targeting one or more antigens, wherein the CAR-T cell is deficient in at least one or more antigens to which the at least one or more CARs specifically binds, wherein the one or more antigens is selected from, but not limited to CD5, CD7, CD2, and CD4.

Immune effector cell: A cell capable of binding an antigen and mediating an immune response selective for the antigen. These cells include, but are not limited to, T cells (T lymphocytes), B cells (B lymphocytes), monocytes, macrophages, natural killer (NK) cells, cytotoxic T lymphocytes (CTLs).

Immune related molecule: A molecule identified in any immune cell, whether in a resting (“non-stimulated”) or activated state, and includes any receptor, ligand, cell surface molecules, nucleic acid molecules, polypeptides, variants and fragments thereof.

Inhibition: The activity value of a polypeptide or polynucleotide of the invention relative to the control sample is about 80%, or about 50%, or about 25% to about 1%, or less. Activation is achieved when the activity value of a polypeptide or polynucleotide of the invention relative to the control sample is about 110%, or 150%, or about 200-500%, or about 1000-3000%, or higher.

Inhibitor, Activator, and/or Modulator: Refers to a compound capable of inhibiting, activating, or modulating IL-7R expression, function, or activity. Such compounds may be identified using in vitro and/or in vivo assays. The term “modulator” includes both inhibitors and activators. A modulator may be an antibody or a soluble ligand that binds a protein of interest, small molecule, or the like. Inhibitors may be an agent that, e.g., inhibits expression of a polypeptide or polynucleotide of the invention, binds to such a polypeptide or polynucleotide, partially or totally blocks stimulation or enzymatic activity, decreases, prevents, delays activation, inactivates, desensitizes, or down regulates the activity of a polypeptide or polynucleotide of the invention, e.g., antagonists.

Interleukin-7 receptor (IL-7R): Refers to a receptor present on a cell surface having an amino acid sequence substantially similar to the sequence of the native human IL-7 receptor amino acid sequence, and which are biologically active, in that they are capable of binding IL-7 molecules or transducing a biological signal initiated by an IL-7 molecule binding to a cell. In some embodiments, an IL-7 receptor as described herein may cross-react with anti-IL-7R antibodies raised against IL-7R from natural (i.e., nonrecombinant) sources. The terms “IL-7 receptor” or “IL-7R” include, but are not limited to, analogs or subunits of native proteins that exhibit at least some biological activity in common with IL-7R. As used throughout the specification, the term “mature” means a protein expressed in a form lacking a leader sequence as may be present in full-length transcripts of a native gene. Various bioequivalent protein and amino acid analogs are described in detail herein. In some embodiments, CD127 or IL-7R may be soluble CD127. The native IL-7 receptor alpha chain is known and described in the literature (see, e.g., Goodwin et al. Cell 60:941-951, 1990; GenBank Accession Nos. NP 032398 and NP002176). As used herein, “CD127 ligand” refers to a compound that binds to the IL-7 receptor, such as native IL-7, or may refer to a drug capable of binding to a modified IL-7 receptor as described herein.

Interleukin-2 receptor (IL-2R): Refers to a receptor present on a cell surface having an amino acid sequence substantially similar to the sequence of the native human IL-2 receptor amino acid sequence, and which are biologically active, in that they are capable of binding IL-2 molecules or transducing a biological signal initiated by an IL-2 molecule binding to a cell.

Interleukin-15 receptor (IL-15R): Refers to a receptor present on a cell surface having an amino acid sequence substantially similar to the sequence of the native human IL-15 receptor amino acid sequence, and which are biologically active, in that they are capable of binding IL-15 molecules or transducing a biological signal initiated by an IL-15 molecule binding to a cell.

Modified IL-7R/IL-15R/IL-2R: Refers to a receptor having substantial sequence similarity to a native IL-7R/IL-15R/IL-2R, but that binds to a drug as described herein, rather than its native IL-7/IL-15/IL-2 ligand. A modified IL-7R/IL-15R/IL-2R may be substantially similar in nucleic acid or protein sequence to a native IL-7R/IL-15R/IL-2R, or may differ significantly in sequence, as long as the modified IL-7R/IL-15R/IL-2R retains function as a receptor and is capable of activating IL-7/IL-15/IL-2 signaling in a cell.

Modulation: Refers to an increase (stimulation) or a decrease (inhibition) in the expression of a gene. This includes any amounts, functions, or the like, as compared to normal controls. The term includes, for example, increased, enhanced, agonized, promoted, decreased, reduced, suppressed, blocked, or antagonized. Modulation can increase activity or amounts more than 1-fold, 2-fold, 3-fold, 5-fold, 10-fold, 100-fold, or the like, over baseline values. Modulation can also decrease its activity or amounts below baseline values.

Nucleotide sequence: A heteropolymer of deoxyribonucleotides. DNA sequences encoding the proteins of the present disclosure may be assembled from cDNA fragments and short oligonucleotide linkers, or from a series of oligonucleotides, to provide a synthetic gene capable of being expressed in a recombinant transcriptional unit.

Recombinant: A protein that is derived from recombinant (e.g., microbial or mammalian) expression systems. “Microbial” refers to recombinant proteins made in bacterial or fungal (e.g., yeast) expression systems. As a product, “recombinant microbial” defines a protein produced in a microbial expression system which is essentially free of native endogenous substances. Protein expressed in most bacterial cultures, e.g., E. coli, will be free of glycan. Protein expressed in yeast may have a glycosylation pattern different from that expressed in mammalian cells.

Recombinant Fusion Protein: A recombinant fusion protein (alternately referred to herein as a chimeric protein, fusion protein, or modified protein) is a protein created through the joining of two or more genes, e.g., through genetic engineering of a fusion gene, that originally coded for separate proteins.

Recombinant Microbial Expression System: A substantially homogenous monoculture of suitable host microorganisms, for example, bacteria such as E. coli or yeast such as S. cerevisiae, which have stably integrated a recombinant transcriptional unit into chromosomal DNA or carry the recombinant transcriptional unit as a component of a resident plasmid. Generally, cells constituting the system are the progeny of a single ancestral transformant. Recombinant expression systems as defined herein will express heterologous protein upon induction of the regulatory elements linked to the DNA sequence or synthetic gene to be expressed.

Safe and Effective Amount or Therapeutic Amount: The quantity of a component sufficient to yield a desired therapeutic response without undue adverse side effects (e.g., toxicity, irritation, or allergic response) commensurate with a reasonable benefit/risk ratio when used in the manner of the present disclosure.

Specific (or Selective) Binding: A binding reaction relating to a protein that is determinative of the presence of the protein, often in a heterogeneous population of proteins and/or other compounds or molecules. For example, specific binding to an antibody under such conditions requires an antibody that is selected for its specificity for a particular protein.

scFv: Refers to a single-chain variable fragment, which is a fusion protein of the variable regions of the heavy and light chains of an immunoglobulin. In some embodiments, a short linker peptide of about 10 to about 25 amino acids may also be included. A linker as described herein may be rich in glycine residues to aid in flexibility of the linker, and may also be rich in serine and/or threonine residues to aid in solubility. A linker may connect the N-terminus of the heavy chain with the C-terminus of the light chain, or may connect the C-terminus of the heavy chain with the N-terminus of the light chain. In some embodiments, a scFv retains the specificity of the original immunoglobulin.

Specifically Modulates: Modulating the activity or function of an IL-7 receptor or its signaling. IL-7Rα chain, or functional domains or peptide sequences, as opposed to any other molecule.

Soluble Protein: a soluble protein, as used herein, is typically one that is administered to a patient. Certain soluble proteins are drugs.

Splice variants: Products of alternative splicing of a gene. After transcription, an initial nucleic acid transcript may be spliced such that different (alternate) nucleic acid splice products encode different polypeptides. Mechanisms for the production of splice variants vary, but may include alternate splicing of exons. Alternate polypeptides derived from the same nucleic acid by read-through transcription are also encompassed by this definition. Any products of a splicing reaction, including recombinant forms of the splice products, are included in this definition. Thus, when referring to, for example, IL-7, or IL-7 receptor, the terms include all variants encompassed by this definition.

Substantially Similar: When used to define either amino acid or nucleic acid sequences, means that a particular subject sequence, for example, a mutant sequence, varies from a reference sequence by one or more substitutions, deletions, or additions, the net effect of which is to retain biological activity of the IL-7R protein, i.e., the ability of the IL-7R to bind to a ligand such as IL-7 or a drug as described herein. Alternatively, nucleic acid subunits and analogs are substantially similar to the specific DNA sequences disclosed herein if: (a) the DNA sequence is derived from the coding region of a native mammalian IL-7R gene; (b) the DNA sequence is capable of hybridization to DNA sequences of (a) under moderately stringent conditions and which encode biologically active IL-7R molecules; or DNA sequences which are degenerate as a result of the genetic code to the DNA sequences defined in (a) or (b) and which encode biologically active IL-7R molecules. Substantially similar analog proteins will be greater than about 30 percent similar to the corresponding sequence of the native IL-7R. Sequences having lesser degrees of similarity but comparable biological activity are c0008-101-USonsidered to be equivalents. In some embodiments, the analog proteins may be greater than about 80 percent similar to the corresponding sequence of the native IL-7R, in which case they are defined as being “substantially identical.” In defining nucleic acid sequences, all subject nucleic acid sequences capable of encoding substantially similar amino acid sequences are considered substantially similar to a reference nucleic acid sequence. Percent similarity may be determined, for example, by comparing sequence information using the GAP computer program, version 6.0, available from the University of Wisconsin Genetics Computer Group (UWGCG). The GAP program utilizes the alignment method of Needleman and Wunsch (J Mol Biol 48:443, 1970), as revised by Smith and Waterman (Adv Appl Math 2:482, 1981). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess, Nucl Acids Res 14:6745, 1986, as described by Schwartz and Dayhoff, ed., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

T cells or T lymphocyte: A subset of lymphocytes originating in the thymus and having receptors (i.e., heterodimeric or homodimeric) associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, activation of other immune cells, such as B-cells) or for the cytokines they produce.

T cell response: An immunological response involving T cells. The T cells that are “activated” divide to produce antigen specific memory T cells or antigen specific cytotoxic T cells. The cytotoxic T cells bind to and destroy cells recognized as containing the antigen. The memory T cells are activated by the antigen and thus provide a response to an antigen already encountered. This overall response to the antigen is the antigen specific T cell response, e.g. tumor specific.

Targeting or Targets: Refers to the specificity of a molecule or other molecule, such as a protein. In some embodiments, specificity or targeting may also refer to the specific functions e.g., signaling of IL-7R alpha chain containing molecules, activity, specific binding of a molecule to the IL-7R, etc.

Therapeutically effective amount: An amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

Transmembrane domain: (TD), refers to a domain on a protein that spans a membrane, such as a plasma membrane. As used herein, a transmembrane domain may refer to a domain on an IL-7 receptor, including a modified IL-7R, found on a specific cell type, such as a T cell or a CAR-T cell. Transmembrane domains are found on transmembrane receptors, which refer to a protein or polypeptide that spans the plasma membrane of a cell, with an extracellular domain of the protein having the ability to bind to a ligand and the intracellular domain having a specific activity that may be altered upon ligand binding. As used herein, a modified IL-7 receptor is a transmembrane receptor that binds to a drug instead of the native IL-7 ligand to activate IL-7 signaling. In some embodiments, a transmembrane receptor may be referred to as a protein or protein complex having a transmembrane domain, and which is capable of binding a ligand (e.g., a drug) to activate IL-7 signaling in a cell.

Variant: A peptide or polypeptide that differs in amino acid sequence by the insertion, deletion, or conservative substitution of amino acids, but retains at least some or a portion of its biological activity; and, when used in the context of an oligonucleotide, means an oligonucleotide that differs in nucleotide sequence by the insertion, deletion, or substitution of nucleotides. A particular nucleic acid sequence also implicitly encompasses splice variants and allelic variants. Similarly, a particular protein encoded by a nucleic acid implicitly encompasses any protein encoded by a splice variant or allelic variant of that nucleic acid.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a,” “an,” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive. Furthermore, to the extent that the terms “including,” “includes,” “having,” “has,” “with,” or variants thereof are used in either the detailed description and/or the claims, such terms are intended to be inclusive in a manner similar to the term “comprising.”

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1. Construction of Recombinant IL-7R, IL-15R, or IL-2R

As shown in FIG. 2, a recombinant IL-7Rα chain may comprise a domain A scFv, linked to a transmembrane domain (including, but not limited to, a transmembrane domain of IL-7Rα or CD28), which in turn may be coupled to an intracellular portion of an IL-7Rα chain. A recombinant common γ chain may comprise a domain B scFv linked to a transmembrane domain (including, but not limited to, a transmembrane domain of the common γ chain or CD28), which in turn may be coupled to an intracellular portion of a common γ chain.

A recombinant protein comprising both domain A scFv and domain B scFv may be a recombinant human protein deactivated such that it does not exhibit natural function, which may or not be modified to increase stability, binding efficacy, inhibition of natural functions, specificity, immunogenicity, and/or half-life. Examples of such a human protein may include, but are not limited to, insulin or osteopontin.

As shown in FIG. 3, administration of a chemically induced dimerization agent, such as FK1012, may lead to dimerization of the IL-7Rα chain and the common γ chain, thus inducing IL-7R signaling. A recombinant IL-7Rα chain may comprise a transmembrane domain (including, but not limited to, a transmembrane domain of IL-7Rα or CD28), linked to a binding domain, such as an FKBP binding domain, which in turn may be coupled to an intracellular portion of an IL-7Rα chain. The recombinant common gamma chain may comprise a transmembrane domain (including, but not limited to, a transmembrane domain of a common gamma chain or CD28), linked to an FKBP binding domain, which in turn may be coupled to an intracellular portion of a common γ chain. In some embodiments, the recombinant IL-7Rα chain and/or the common γ chain may also include an extracellular peptide tag, for example, human truncated CD34. In some embodiments, the position of an FKBP domain may alternatively be located extracellularly, or at the terminal of the intracellular signaling domain distal to the plasma membrane.

In some embodiments, recombinant IL-15R and IL-2R may be constructed in a similar manner, by modifying the IL-15Rα chain, the IL-15Rβ chain, or the common γ chain, which make up the IL-15R, or by modifying the IL-2Rα chain, the IL-2Rβ chain, or the common γ chain, which make up the IL-2R.

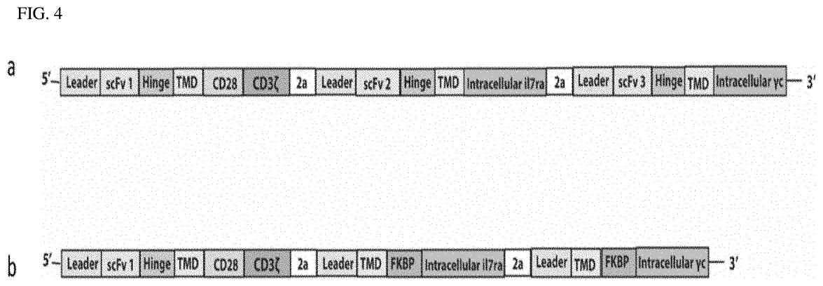

A modified or recombinant receptor of the present disclosure may be expressed as individual nucleic acid molecules encoding a single receptor chain or subunit each. Alternatively, the chains of a modified receptor could be expressed as single nucleic acid molecule separated by, e.g., a 2A peptide, an IRES element, or other expression systems that result in polycistronic expression. Examples of polycistronic DNA constructs designed to express a CAR, a recombinant IL-7Rα chain, and a recombinant common γ chain as three independent proteins are shown in FIG. 4. In part (a), an intracellular domain of an IL-7Rα chain is fused to an scFv via a linker protein (hinge) and a transmembrane domain (TMD). The intracellular γc domain is fused to an scFv via a linker protein (hinge) and a TMD. A leader sequence is included for trafficking of individual chains to the cell membrane. 2A peptides separate a CAR, recombinant IL-7Rα chain, and recombinant γc produce independent proteins. In part (b), an intracellular domain of an IL-7Rα chain is fused to an FKBP domain and a TMD. An intracellular γc domain is fused to a FKBP domain and a TMD. A leader sequence is included for trafficking of individual chains to the cell membrane. 2A peptides separate the CAR, recombinant IL-7Rα chain, and recombinant γc to produce independent proteins.

### Example 2. Binding Assays

Radiolabeling of IL-7, IL-15, or IL-2—Recombinant murine or other IL-7, IL-15, or IL-2 is expressed in host cells and purified. The purified protein is labeled using commercially available means. The final pool of labeled IL-7, IL-15, or IL-2 is diluted to a working stock solution in a suitable medium.

Binding of Signaling Peptide, Soluble Protein, or Drug to Intact Cells—Binding assays are performed to determine binding of a peptide capable of inducing signaling in cells having a modified IL-7R, IL-15R, or IL-2R as described herein. Such assays are performed in cells grown in suspension culture or removed from culture flasks. Additional binding assays may also be done to show that endogenous IL-7, IL-15, or IL-2 does not bind to a modified receptor as described herein. Monolayers may be used for such assays, or solid phase binding assays may also be done.

Binding Assay for Soluble IL-7R, IL-15R, or IL-2R-Soluble IL-7, IL-15, or IL-2 receptor present in cell supernatants is measured by inhibition of IL-7, IL-15, or IL-2 binding to an IL-7-, IL-15-, or IL-2-dependent cell line or any cell line expressing IL-7, IL-15, or IL-2 receptors. Supernatants are harvested from cells, concentrated, and preincubated with IL-7, IL-15, or IL-2, and binding is assayed.

Administration of an scFv, a small molecule agonist, or an antibody targeting the stimulating protein could be used to saturate the protein and block recombinant IL-7R, IL-15R, or IL-2R signaling. The two chains of the recombinant IL-7R, IL-15R, or IL-2R could be expressed from the same vector as the CAR. The distinct chains could be expressed independently (from each other as well as the CAR), from distinct plasmids, mRNA, viral vectors, (or other non-viral systems previously described, i.e., targeted insertion into the genome). Alternatively, the distinct chains could be expressed as single nucleic acid molecule separated by, e.g., a 2A peptide, an IRES element, or other expression systems that result in polycistronic expression.

### Example 3. Isolation of IL-7, IL-15, or IL-2 Receptor cDNA

Various cell lines are screened for expression of IL-7, IL-15, or IL-2 receptor based on their ability to bind labeled IL-7, IL-15, or IL-2, respectively. A sized cDNA library is constructed by reverse transcription of polyadenylated mRNA isolated from total RNA extracted from cells using standard techniques. The cells are harvested by lysing the cells and total RNA is isolated.

Poly A+ RNA is isolated and double-stranded cDNA is prepared. For example, the poly A+ RNA may be converted to an RNA-cDNA hybrid by reverse transcriptase using oligo dT as a primer. The RNA-cDNA hybrid may then be converted into double-stranded cDNA using RNAase H in combination with DNA polymerase I. The resulting double stranded cDNA may then be blunt-ended with T4 DNA polymerase. EcoRI linker-adapters may be added to the blunt-ended cDNA. The linker-adapted cDNA may then be treated with T4 polynucleotide kinase to phosphorylate the 5′ overhanging region of the linker-adapter and unligated linkers may be removed. The linker-adapted cDNA may then be ligated to a preferred vector, for example bacteriophage λgt10 and packaged into phage particles to generate a library of recombinants. Recombinants may then be further amplified by plating the phage on a bacterial lawn of E. coli. Phage DNA may be purified from the resulting λgt10 cDNA library and the cDNA inserts excised by digestion. Following electrophoresis of the digest through an agarose gel, cDNAs greater than 500 bp may be isolated. The resulting cDNAs may be ligated into a eukaryotic expression vector, for example one that is designed to express cDNA sequences inserted at its multiple cloning site when transfected into mammalian cells. In addition, a sequence obtained from a public database and then modified may be commercially synthesized into, for example, a lentiviral vector.

The resulting cDNA library is used to transform E. coli, and recombinants are plated. Colonies are scraped from each plate, pooled, and plasmid DNA prepared from each pool. The pooled DNA is then used to transfect cells. The cells are then grown in culture to permit transient expression of the inserted sequences. Cell culture supernatants are discarded after culture and the cell monolayers in each plate assayed for IL-7, IL-15, or IL-2 binding.

Frozen bacterial stock from positive pools are then used to obtain plates of colonies. Replicas of plates may also be made. Plates are scraped and plasmid DNA prepared and transfected to identify a positive plate. Bacteria from individual colonies are grown in cultures, which are used to obtain plasmid DNA, which is then transfected into cells. Clones capable of inducing expression of IL-7, IL-15, or IL-2 receptor in cells is then identified. The cDNA insert is subcloned into a plasmid.

### Example 4. Binding Characteristics of Human IL-7, IL-15, or IL-2 Receptors

The various clones isolated or synthesized above are analyzed using binding assays as described in Example 1.

Human IL-7R, IL-15R, or IL-2R is analyzed by transfecting cells with a human IL-7, IL-15, or IL-2 receptor clone, respectively, as described in Example 1. Numbers of high and low affinity sites per cell are determined in cells expressing the human IL-7, IL-15, or IL-2 receptor clone. The results demonstrate that the binding characteristics of recombinant human IL-7R, IL-15R, or IL-2R expressed in cells are very similar to those of the naturally occurring receptors found on cells.

The binding characteristics of the cDNA-encoded receptor molecule are also analyzed. For example, it can be determined whether a particular clone lacks the putative transmembrane domain and is secreted from cells. Cell supernatants of the cells is tested as described in Example 1 to determine if binding of labeled IL-7, IL-15, or IL-2 to the IL-7, IL-15, or IL-2 receptors present on cells is inhibited. Preincubation of the 125 I-labeled IL-7, IL-15, or IL-2 with conditioned media from cells transfected with cDNA is done to determine whether it results in the subsequent inhibition of binding of labeled IL-7, IL-15, or IL-2 to cells.

### Example 5. Preparation of Monoclonal Antibodies to IL-7, IL-15, or IL-2 Receptor

Preparations of purified recombinant IL-7, IL-15, or IL-2 receptor, for example, human IL-7R, IL-15R, or IL-2R, or transfected cells expressing high levels of IL-7, IL-15, or IL-2 receptor are employed to generate monoclonal antibodies against IL-7R, IL-15R, or IL-2R using conventional techniques, for example, those disclosed in U.S. Pat. No. 4,411,993. Such antibodies are likely to be useful in interfering with IL-7, IL-15, or IL-2 binding to IL-7, IL-15, or IL-2 receptors, respectively, for example, in ameliorating toxic or other undesired effects of IL-7, IL-15, or IL-2, or as components of diagnostic or research assays for IL-7, IL-15, or IL-2, or soluble IL-7, IL-15, or IL-2 receptor.

To immunize mice, IL-7R, IL-15R, or IL-2R immunogen is emulsified in complete Freund's adjuvant and injected in amounts ranging from 10-100 μg subcutaneously into Balb/c mice. Ten to twelve days later, the immunized animals are boosted with additional immunogen emulsified in incomplete Freund's adjuvant and periodically boosted thereafter on a weekly to biweekly immunization schedule. Serum samples are periodically taken by retro-orbital bleeding or tail-tip excision for testing by dot-blot assay (antibody sandwich) or ELISA (enzyme-linked immunosorbent assay). Other assay procedures are also suitable. Following detection of an appropriate antibody titer, positive animals are given an intravenous injection of antigen in saline. Three to four days later, the animals are sacrificed, splenocytes harvested, and fused to the murine myeloma cell line NS1. Hybridoma cell lines generated by this procedure are plated in multiple microtiter plates in a HAT selective medium (hypoxanthine, aminopterin, and thymidine) to inhibit proliferation of non-fused cells, myeloma hybrids, and spleen cell hybrids.

Hybridoma clones thus generated can be screened by ELISA for reactivity with IL-7, IL-15, or IL-2 receptor. Positive clones are then injected into the peritoneal cavities of syngeneic Balb/c mice to produce ascites containing high concentrations (>1 mg/ml) of anti-IL-7, IL-15, or IL-2 receptor monoclonal antibody. The resulting monoclonal antibody can be purified by ammonium sulfate precipitation followed by gel exclusion chromatography, and/or affinity chromatography based on binding of antibody to Protein A of Staphylococcus aureus.

### Example 6. CAR-Bearing Immune Effector Cells

Several types of genome-edited CAR-bearing immune effector cells comprising a chimeric IL-7, IL-15, or IL-2 receptor may be made and are disclosed herein. For example, a CAR-T cell may have a single CAR that recognizes a single antigen, referred to herein as a mono-CAR-T cell. Examples of such mono-CAR-T cells are provided below in Table 1, with and without deletion or suppression of one or more surface proteins that is/are the antigen targets of the CARs. In general, examples with deletion or suppression of more antigens will be more likely to have the benefit of greater fratricide resistance.

Mono-CAR-T cells are generated from a suitable source of T cells, such as blood from a subject. Blood cells are collected, isolated, enriched, and expanded ex vivo before transduction with a CAR to generate a CAR-T cell.

To generate a mono-CAR-T cell, a single chain variable fragment (scFv) of interest, such as one listed in Table 1, is commercially synthesized and cloned into a CAR backbone. CD28 and 4-1BB internal signaling domains can be included as co-stimulatory domains. To prevent fratricide, the same target of interest is deleted in CAR-T using, for example, CRISPR/Cas9 or TALEN gene-editing (Table 1). Alternatively, modification of the TCR may be performed such that the CAR-T cell is deficient in endogenous T cell receptor (TCR) signaling. To accomplish this, the antigen of the T cell may be modified such that the CAR no longer specifically binds the modified antigen. For example, the epitope of the antigen recognized by the CAR may be modified by one or more amino acid changes (e.g., substitutions or deletions), or the epitope may be deleted from the antigen. In other embodiments, expression of the antigen is reduced in the T cell. Alternatively, the T cell is modified such that the antigen is not expressed, e.g., by deletion or disruption of the gene encoding the antigen. For example, CRISPR/cas9 gene editing can be used to modify a T cell to be deficient in an antigen.

CAR-T cells encompassed by the present disclosure may further be deficient in endogenous T cell receptor (TCR) signaling. For example, decreasing or eliminating endogenous TCR signaling in CAR-T cells may prevent or reduce graft versus host disease (GvHD) when allogenic T cells are used to produce the CAR-T cells. Methods for decreasing or eliminating endogenous TCR signaling are known in the art and include, but are not limited, to modifying a part of the TCR receptor (e.g., the TCR receptor alpha chain (TRAC), etc.). TRAC modification may block TCR mediated signaling. TRAC modification may thus permit the safe use of allogeneic T cells as the source of CAR-T cells without inducing life-threatening GvHD.

Alternatively, or in addition, CAR-T cells encompassed by the present disclosure may further comprise one or more suicide genes. As used herein, “suicide gene” refers to a nucleic acid sequence introduced to a CAR-T cell by standard methods known in the art that, when activated, results in the death of the CAR-T cell. Suicide genes may facilitate effective tracking and elimination of the CAR-T cells in vivo if required. Facilitated killing by activating the suicide gene may occur by methods known in the art. Suitable suicide gene therapy systems known in the art include, but are not limited to, various the herpes simplex virus thymidine kinase (HSV-tk)/ganciclovir (GCV) suicide gene therapy systems or inducible caspase 9 protein. In an exemplary embodiment, a suicide gene is a CD34/thymidine kinase chimeric suicide gene.

Following gene editing, transduction of the T cells with the CAR-T construct is performed to generate the CAR-T cells.

### Example 7. Tandem or Dual CAR-T Cells

Additionally, several types of dual or tandem CAR-T cells may be made and are disclosed herein. Examples of tandem and dual CAR-T cells are provided below in Table 2, with and without deletion or suppression of one or more surface proteins that is/are the antigen targets of the CARs. In general, examples with deletion or suppression of more antigens will be more likely to have the benefit of greater fratricide resistance. It should be further noted that the order in which the antigens (scFv) are oriented in the tandem CARs set forth below in Table 1 is not meant to be limiting and includes tandem CAR-T cells in either orientation. For example, the CD2*CD3ε encompasses a tCAR with the orientation CD2*CD3ε or one with the orientation CD3ε*CD2.

### Example 8. iNKT-CAR Cells

Several types of iNKT cells may be made as described herein. In general, examples with deletion or suppression of antigens will have the benefit of fratricide resistance. In certain embodiments, the iNKT-CAR has deletion or suppression of the surface protein that is the antigen target of the chimeric antigen receptor.

Additional examples of tandem and dual iNKT-CARs are provided below, with and without deletion or suppression of one or more surface proteins that is/are the antigen targets of the CARs. In general, examples with deletion or suppression of more antigens will be more likely to have the benefit of greater fratricide resistance. It should be further noted that the order in which the antigens (scFv) are oriented in the tandem CARs set forth below in Table 4 is not meant to be limiting, and includes tandem iNKT-CARs in either orientation. For example, the CD2×CD3ε iNKT-tCAR is encompasses a tCAR with the orientation CD2-CD3ε or one with the orientation CD3ε-CD2. Finally, it should also be noted that because NK cells do not express a TCR, and iNKT cells express an invariant TCR that recognizes glycolipids instead of peptides and proteins, inactivation of the TCR or its subparts (e.g., through deletion or suppression of TRAC/TCRα, TCRβ, or CD3ε) is typically not necessary to prevent graft-vs-host disease when using allogeneic cells.

Antigens which the CARs, to be used in the CAR-bearing immune effector cells comprising the chimeric IL-7, IL-15, or IL-2 receptors disclosed herein, may bind, include but are not limited to antigens present on the surface of lymphomas and leukemias including B-cell cancers, T-cell cancers, and multiple myeloma. Table 1 below lists examples of CAR-T, CAR-iNKT, or CAR-NK cells which

TABLE 1

| | | CAR-T, CAR-iNKT, or | Mono, |
|---|---|---|---|
| CAR | Antigen Target | CAR-NK Cell Antigen | Tandem or |
| Example | of CAR | Deletion/Suppression | Dual CAR |
| 1 | CD2 | — | Mono |
| 2 | CD2 | CD2 | Mono |
| 3 | CD3ε | — | Mono |
| 4 | CD3ε | CD3ε | Mono |
| 5 | CD4 | — | Mono |
| 6 | CD4 | CD4 | Mono |
| 7 | CD5 | — | Mono |
| 8 | CD5 | CD5 | Mono |
| 9 | CD7 | — | Mono |
| 10 | CD7 | CD7 | Mono |
| 11 | TCRβ | — | Mono |
| 12 | TCRβ | TCRβ | Mono |
| 13 | APRIL | — | Mono |
| 14 | BCMA | — | Mono |
| 15 | CD19 | — | Mono |
| 16 | CD38 | — | Mono |
| 17 | CD38 | CD38 | Mono |
| 18 | CS1 | — | Mono |
| 19 | CS1 | CS1 | Mono |
| 20a/b | CD2xCD3ε | — | Dual/Tandem |
| 21a/b | CD2xCD3ε | CD2 | Dual/Tandem |
| 22a/b | CD2xCD3ε | CD2 and CD3ε | Dual/Tandem |
| 23a/b | CD2xCD3ε | CD3ε | Dual/Tandem |
| 24a/b | CD2xCD4 | — | Dual/Tandem |
| 25a/b | CD2xCD4 | CD2 | Dual/Tandem |
| 26a/b | CD2xCD4 | CD2 and CD4 | Dual/Tandem |
| 27a/b | CD2xCD4 | CD4 | Dual/Tandem |
| 28a/b | CD2xCD5 | — | Dual/Tandem |
| 29a/b | CD2xCD5 | CD2 | Dual/Tandem |
| 30a/b | CD2xCD5 | CD2 and CD5 | Dual/Tandem |
| 31a/b | CD2xCD5 | CD5 | Dual/Tandem |
| 32a/b | CD2xCD7 | — | Dual/Tandem |
| 33a/b | CD2xCD7 | CD2 | Dual/Tandem |
| 34a/b | CD2xCD7 | CD2 and CD7 | Dual/Tandem |
| 35a/b | CD2xCD7 | CD7 | Dual/Tandem |
| 36a/b | CD3εxCD4 | — | Dual/Tandem |
| 37a/b | CD3εxCD4 | CD3ε | Dual/Tandem |
| 38a/b | CD3εxCD4 | CD3ε and CD4 | Dual/Tandem |
| 39a/b | CD3εxCD4 | CD4 | Dual/Tandem |
| 40a/b | CD3εxCD5 | — | Dual/Tandem |
| 41a/b | CD3εxCD5 | CD3ε | Dual/Tandem |
| 42a/b | CD3εxCD5 | CD3ε and CD5 | Dual/Tandem |
| 43a/b | CD3εxCD5 | CD5 | Dual/Tandem |
| 44a/b | CD3εxCD7 | — | Dual/Tandem |
| 45a/b | CD3εxCD7 | CD3ε | Dual/Tandem |
| 46a/b | CD3εxCD7 | CD3ε and CD7 | Dual/Tandem |
| 47a/b | CD3εxCD7 | CD7 | Dual/Tandem |
| 48a/b | CD4xCD5 | — | Dual/Tandem |
| 49a/b | CD4xCD5 | CD4 | Dual/Tandem |
| 50a/b | CD4xCD5 | CD4 and CD5 | Dual/Tandem |
| 51a/b | CD4xCD5 | CD5 | Dual/Tandem |
| 52a/b | CD4xCD7 | — | Dual/Tandem |
| 53a/b | CD4xCD7 | CD4 | Dual/Tandem |
| 54a/b | CD4xCD7 | CD7 | Dual/Tandem |
| 55a/b | CD5xCD7 | — | Dual/Tandem |
| 56a/b | CD5xCD7 | CD5 | Dual/Tandem |
| 57a/b | CD5xCD7 | CD5 and CD7 | Dual/Tandem |
| 58a/b | CD5xCD7 | CD7 | Dual/Tandem |
| 59a/b | TCRβxCD2 | — | Dual/Tandem |
| 60a/b | TCRβxCD2 | CD2 | Dual/Tandem |
| 61a/b | TCRβxCD2 | TCRβ | Dual/Tandem |
| 62a/b | TCRβxCD2 | TCRβ and CD2 | Dual/Tandem |
| 63a/b | TCRβxCD3 | TCRβ and CD3 | Dual/Tandem |
| 64a/b | TCRβxCD3ε | — | Dual/Tandem |
| 65a/b | TCRβxCD3ε | CD3ε | Dual/Tandem |
| 66a/b | TCRβxCD3ε | TCRβ | Dual/Tandem |
| 67a/b | TCRβxCD3ε | TCRβ and CD3ε | Dual/Tandem |
| 68a/b | TCRβxCD4 | — | Dual/Tandem |
| 69a/b | TCRβxCD4 | CD4 | Dual/Tandem |
| 70a/b | TCRβxCD4 | TCRβ | Dual/Tandem |
| 71a/b | TCRβxCD4 | TCRβ and CD4 | Dual/Tandem |
| 72a/b | TCRβxCD5 | — | Dual/Tandem |
| 73a/b | TCRβxCD5 | CD5 | Dual/Tandem |
| 74a/b | TCRβxCD5 | TCRβ | Dual/Tandem |
| 75a/b | TCRβxCD5 | TCRβ and CD5 | Dual/Tandem |
| 76a/b | TCRβxCD7 | — | Dual/Tandem |
| 77a/b | TCRβxCD7 | CD7 | Dual/Tandem |
| 78a/b | TCRβxCD7 | TCRβ | Dual/Tandem |
| 79a/b | TCRβxCD7 | TCRβ and CD7 | Dual/Tandem |
| 80a/b | TRAC | — | Dual/Tandem |
| 81a/b | TRACxCD2 | — | Dual/Tandem |
| 82a/b | TRACxCD2 | CD2 | Dual/Tandem |
| 83a/b | TRACxCD2 | TRAC | Dual/Tandem |
| 84a/b | TRACxCD2 | TRAC and CD2 | Dual/Tandem |
| 85a/b | TRACxCD3 | TRAC and CD3 | Dual/Tandem |
| 86a/b | TRACxCD3ε | — | Dual/Tandem |
| 87a/b | TRACxCD3ε | CD3ε | Dual/Tandem |
| 88a/b | TRACxCD3ε | TRAC | Dual/Tandem |
| 89a/b | TRACxCD3ε | TRAC and CD3ε | Dual/Tandem |
| 90a/b | TRACxCD4 | — | Dual/Tandem |
| 91a/b | TRACxCD4 | CD4 | Dual/Tandem |
| 92a/b | TRACxCD4 | TRAC | Dual/Tandem |
| 93a/b | TRACxCD4 | TRAC and CD4 | Dual/Tandem |
| 94a/b | TRACxCD5 | — | Dual/Tandem |
| 95a/b | TRACxCD5 | CD5 | Dual/Tandem |
| 96a/b | TRACxCD5 | TRAC | Dual/Tandem |
| 97a/b | TRACxCD5 | TRAC and CD5 | Dual/Tandem |
| 98a/b | TRACxCD7 | — | Dual/Tandem |
| 99a/b | TRACxCD7 | CD7 | Dual/Tandem |
| 100a/b | TRACxCD7 | TRAC | Dual/Tandem |
| 101a/b | TRACxCD7 | TRAC and CD7 | Dual/Tandem |
| 102a/b | APRILxBCMA | — | Dual/Tandem |
| 103a/b | APRILxCD19 | — | Dual/Tandem |
| 104a/b | APRILxCD38 | — | Dual/Tandem |
| 105a/b | APRILxCD38 | CD38 | Dual/Tandem |
| 106a/b | APRILxCS1 | — | Dual/Tandem |
| 107a/b | APRILxCS1 | CS1 | Dual/Tandem |
| 108a/b | BCMA | — | Dual/Tandem |
| 109a/b | BCMA | — | Dual/Tandem |
| 110a/b | BCMAxCD19 | — | Dual/Tandem |
| 111a/b | BCMAxCD38 | — | Dual/Tandem |
| 112a/b | BCMAxCD38 | CD38 | Dual/Tandem |
| 113a/b | BCMAxCS1 | — | Dual/Tandem |
| 114a/b | BCMAxCS1 | CS1 | Dual/Tandem |
| 115a/b | CD19 | — | Dual/Tandem |
| 116a/b | CD19xCD38 | — | Dual/Tandem |
| 117a/b | CD19xCD38 | CD38 | Dual/Tandem |
| 118a/b | CD38 | — | Dual/Tandem |
| 119a/b | CD38 | CD38 | Dual/Tandem |
| 120a/b | CS1 | — | Dual/Tandem |
| 121a/b | CS1 | CS1 | Dual/Tandem |
| 122a/b | CS1xCD19 | — | Dual/Tandem |
| 123a/b | CS1xCD19 | CS1 | Dual/Tandem |
| 124a/b | CS1xCD38 | — | Dual/Tandem |
| 125a/b | CS1xCD38 | CD38 | Dual/Tandem |
| 126a/b | CS1xCD38 | CS1 | Dual/Tandem |
| 127a/b | CS1xCD38 | CS1 and CD38 | Dual/Tandem |

### Example 9. Chimeric IL-7 Receptors

The intracellular domain (ICD; endodomain) and transmembrane domain (TMD) of the IL-7 receptor alpha chain (IL-7Ra) containing the components required for signaling related to CAR-T and T cell homeostasis and activation (Carrette and Surh, Seminars in Immunology 2012) is hybridized to an interchangeable extracellular domain (ECD; ectodomain) of an unrelated protein linked by a CD8 linker domain. This unrelated protein could be, e.g., an antibody or binding portion thereof, such as an Fab, scFv, F(ab′)₂, minibody, or other binding arrangement of V_{H} and V_{L} chains or CDR-containing proteins.

Expression of this chimeric protein allows for the precise control of IL-7 signaling in therapeutic CAR-T cells allowing for increased CAR-T activation and persistence independent of endogenous IL-7 cytokine production. Unlike previous attempts to bolster CAR-T activity using mutant IL-7 receptors which are constitutively active (“always on”) (Zenatti et al., Nature Genetics 2011), this approach allows for a calibrated approach, and therefore decreases the expected toxicity inherent to a constitutively active IL-7 receptor.

The previous attempts at modulating IL-7 signaling via the addition of a cysteine residue within the IL-7Ra TMD results in the formation of a disulfide bridge resulting in the homodimerization of IL-7Ra and signal transduction independent of any ligand binding; this results in the constitutively active IL-7 receptor.

The current approach allows titration in of an independent ligand in the form of a biologic, or other macromolecule (e.g., FDA approved), to activate IL-7 signaling in the target cell. This has the advantage of decreased toxicity, clinically controllable activation schemes, and the ability to vary the input ligand corresponding to our interchangeable ECD.

DNA Construct design: Below are DNA designs for chimeric IL-7 receptors. The complete gene may be expressed in a lentiviral plasmid vector (pLVM-EF1a, e.g., Addgene) allowing for the generation of lentiviruses capable of transducing therapeutic CAR-T cells with the construct(s).

Construct 1: Human IgE CE3 IL-7Ra chimera “WU44”.

Domain 1: Kozak and CD8a signal peptide
(SEQ ID NO: 1)
GCCACCatggccttaccagtgaccgccttgctcctgccgctggccttgc
tgctccacgccgccaggccg
Domain 2: Human IgE CE3 ECD
(SEQ ID NO: 2)
Tccaacccgagaggggtgagcgcctacctaagccggcccagcccgttcg
acctgttcatccgcaagtcgcccacgatcacctgtctggtggtggacct
ggcacccagcaaggggaccgtgaacctgacctggtcccgggccagtggg
aagcctgtgaaccactccaccagaaaggaggagaagcagcgcaatggca
cgttaaccgtcacgtccaccctgccggtgggcacccgagactggatcga
gggggagacctaccagtgcagggtgacccacccccacctgcccagggcc
ctcatgcggtccacgaccaagaccagc
Domain 3: CD8 Linker ECD
(SEQ ID NO: 3)
Accacgacgccagcgccgcgaccaccaacaccggcgcccaccatcgcgt
cgcagcccctgtccctgcgcccagaggcgtgccggccagcggcgggggg
cgcagtgcacacgagggggctggacttcgcctgtgat
Domain 4: IL-7Ra TMD
(SEQ ID NO: 4)
Cctatcttactaaccatcagcattttgagttttttctctgtcgctctgt
tggtcatcttggcctgtgtgttatgg
Domain 5: IL-7Ra ICD
(SEQ ID NO: 5)
Aaaaaaaggattaagcctatcgtatggcccagtctccccgatcataaga
agactctggaacatctttgtaagaaaccaagaaaaaatttaaatgtgag
tttcaatcctgaaagtttcctggactgccagattcatagggtggatgac
attcaagctagagatgaagtggaaggttttctgcaagatacgtttcctc
agcaactagaagaatctgagaagcagaggcttggaggggatgtgcagag
ccccaactgcccatctgaggatgtagtcatcactccagaaagctttgga
agagattcatccctcacatgcctggctgggaatgtcagtgcatgtgacg
cccctattctctcctcttccaggtccctagactgcagggagagtggcaa
gaatgggcctcatgtgtaccaggacctcctgcttagccttgggactaca
aacagcacgctgccccctccattttctctccaatctggaatcctgacat
tgaacccagttgctcagggtcagcccattcttacttccctgggatcaaa
tcaagaagaagcatatgtcaccatgtccagcttctaccaaaaccag
Domain 6: P2A-Thy1.1 co-expressed surface marker
(SEQ ID NO: 6)
Ggctccggagccacgaacttctctctgttaaagcaagcaggagacgtgg
aagaaaaccccggtcccatgggtcttttctgcagtcaccgtcctcgagg
caccatgaacccagccatcagcgtcgctctcctgctctcagtcttgcag
gtgtcccgagggcagaaggtgaccagcctgacagcctgcctggtgaacc
aaaaccttcgcctggactgccgccatgagaataacaccaaggataactc
catccagcatgagttcagcctgacccgagagaagaggaagcacgtgctc
tcaggcacccttgggatacccgagcacacgtaccgctcccgcgtcaccc
tctccaaccagccctatatcaaggtccttaccctagccaacttcaccac
caaggatgagggcgactacttttgtgagcttcgcgtctcgggcgcgaat
cccatgagctccaataaaagtatcagtgtgtatagagacaagctggtca
agtgtggcggcataagcctgctggttcagaacacatcctggatgctgct
gctgctgctttccctctccctcctccaagccctggacttcatttctctg
tga

Construct 2: Anti-NP Clone 3B44 SCFV IL-7Ra Chimera “WU45”

Domain 1: Kozak and CD8a signal peptide
See construct 1
Domain 2: Anti-NP 3B44 SCFV ECD
(SEQ ID NO: 7)
CAGGCTGTTGTGACTCAGGAATCTGCACTCACCACATCACCTGGTGAAA
CAGTCACACTCACTTGTCGCTCAAGTACTGGGGCTGTTACAACTAGTAA
CTATGCCAACTGGGTCCAAGAAAAACCAGATCATTTATTCACTGGTCTA
ATAGGTGGcACCAACAACCGAGCTCCAGGTGTTCCTGCCAGATTCTCAG
GCTCCCTGATTGGAGACAAGGCTGCCCTCACCATCACAGGGGCACAGAC
TGAGGATGAGGCAATATATTTCTGTGCTCTATGGTACAGCAACCACTGG
GTGTTCGGTGGAGGAACCAAACTGACTGTCCTAggtggtggtggttctg
gtggtggtggttctggcggcggcggctccggtggtggtggatccCAGGT
CCAATTGCAGCAGCCTGGGTCTGAGCTTGTGAAGCCTGGGGCTTCAGTC
AAGCTGTCCTGCAAGGCTTCTGGCTACACCTTCACCAGCTACTTGATGC
ACTGGGTGAGGCAGAGGCCTGGACGAGGCCTTGAGTGGATTGGAAGGAT
TGATCCTAATAGTGGTGGTACTAAATACAGTGAGAAGTTGAAGAGCAAG
GCCACACTGACTGTAGACAAACCCTCCAGCACAGCCTACATGCAGCTCA
GGAGCCTGACATCTGAGGACTCTGCGGTCTATTATTGTGCAACATATTA
CTACGGTAGTAGCTACTACTTTGACTACTGGGGCCAAGGCACCACTCTC
ACAGTCTCCTCA
Domain 3: CD8 Linker ECD
See construct 1
Domain 4: IL-7Ra TMD
See construct 1
Domain 5: IL-7Ra ICD
See construct 1
Domain 6: P2A-Thy1.1 co-expressed surface marker
See construct 1
Construct 3: Human Carcinoembryonioc Antigen
(CEA) IL-7Ra chimera “WU46”
Domain 1: Kozak and CD8a signal peptide
See construct 1
Domain 2: CEA a2-b3 ECD
(SEQ ID NO: 8)
Cccaaacccttcatcaccagcaacaattctaaccccgtggaggatgagg
atgctgtagccttaacctgtgaacctgagattcagaacacaacctacct
gtggtgggtaaataatcagagcctcccggtgagtccaagactgcagctg
tctaatgacaacaggaccctcactctactcagtgtcacaaggaatgatg
taggaccctatgagtgtggaatccagaacgaattaagtgttgaccacag
cgacccagtcatcctgaatgtcctctatggcccagacgaccccaccatt
tccccctcatacacctattaccgtccaggggtgaacctcagcctctcct
gccatgcagcctctaacccacctgcacagtattcttggctgattgatgg
gaacatccaacagcatacacaagagctctttatctccaacatcactgag
aagaacagcggactctatacctgccaggccaataactcagccagtggcc
acagcaggactacagtcaagacaatcacagtctctgcggagctgcccaa
gccctccatctccagcaacaactccaaacccgtggaggacaaggatgct
gtggccttcacctgtgaacctgaggctcagaataccacttatctctggt
gggtgaacggtcagagcctcccagtcagtcccaggctgcagctgtccaa
tggcaatagaacactgaccctattcaatgtcacaagaaatgacgcaaga
gcctatgtatgtggaatccagaactcagtgagtgcaaaccgcagtgacc
cagtcaccctggatgtcctctatgggccggacacccccatcatttcccc
cccagactcgtcttacctttcgggagcgaacctcaacctctcctgccac
tcggcctctaacccatccccgcagtattcttggcgtatcaatgggatac
cgcagcaacacacacaagttctctttatcgccaaaatcacgccaaataa
taacgggacctatgcctgttttgtctctaacttggctactggccgcaat
aattccatagtcaagagcatcacagtctctgcatctggaacttctcctg
gtctctcagctggggccactgtcggcatcatgattggagtgctggttgg
ggttgctctgata
Domain 3: CD8 Linker ECD
See construct 1
Domain 4: IL-7Ra TMD
See construct 1
Domain 5: IL-7Ra ICD
See construct 1
Domain 6: P2A-Thy1.1 co-expressed surface marker
See construct 1

Ligands to Activate Chimeric IL-7 Receptor:

Construct 1 (IgE_CE3 IL-7Ra Chimera, “WU44”):

Omalizumab (Xolair®) binds to the C-epsilon 3 (CE3) domain of the human IgE antibody, and is an FDA approved biologic for the treatment of severe allergies or asthma. Omalizumab is an IgG1 monoclonal antibody with bivalent F_{ab} regions, allowing for the homodimerization of WU44 and downstream IL-7 receptor signal propagation.

Construct 2 (Anti-NP 3B44 IL-7Ra Chimera, “WU45”):

4-Hydroxy-3-nitrophenyl (NP) hapten can be conjugated to a large number of proteins at a high NP to protein ratio. In this case, NP is conjugated to bovine serum albumin (BSA). The NP decorated BSA binds to the anti-NP 3B44 SCFV ECD of WU45 causing the homodimerization of WU45 and downstream IL-7 receptor signal propagation.

Construct 3 (CEA IL-7Ra Chimera, “WU46”):

CEA-Scan (arcitumomab) is an FDA approved antibody Fab′ fragment for the use of diagnostic imaging of colorectal cancers. CEA antigen is mainly expressed by human embryos, with limited expression in adult tissues except in the case of certain colorectal cancers. Similar to proposed binding of omalizumab to WU44, arcitumomab (murine IgG1) binding to the CEA ECD of WU46 allows for the homodimerization of WU46 and downstream IL-7 receptor signal propagation.

Methods

DNA Synthesis and Molecular Cloning

DNA constructs WU44, 45, and 46 were synthesized using IDT's gene block platform.

Molecular cloning into the lentiviral pLVM vector uses the New England Biosciences SALI-HF and NOTI restriction enzymes. Cloned constructs are used to transform Agilent XL1 Blue competent bacteria and selected using carbenicillin LB agar plates. Positively selected bacteria containing the target plasmid are expanded in liquid LB culture with carbenicillin and plasmid preparations are made using the Macherey Nagel NucleoBond Xtra midi kits. Plasmids are sequence verified using Sanger sequencing provided by Genewiz.

Surface Expression Testing and Lentiviral Production

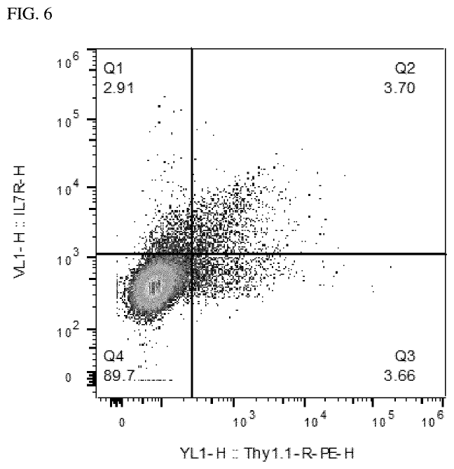

Plasmids are transfected into 293T cells under standard tissue culture conditions using Thermo Fisher Lipofectamine 2000 reagent. Transfection of 293T cells with our constructs will allow validation of surface expression of our chimeric proteins as well as the production of lentivirus for downstream applications (see FIG. 6).

Surface expression of IL-7R chimeric constructs assessed by flow cytometry using omalizumab, NP-BSA, or arcitumomab conjugated to a fluorochrome via the Novus Biologics Lightning Link kit to label WU44, 45, and 46 respectively. Co staining with anti-Thy1.1 antibody is also used to verify protein expression and for potential downstream purification purposes.

Preliminary analysis of expression of WU46 construct on HEK-293T cells was performed, in which HEK-293T cells were transfected using lipofectamine 2000 (ThermoFisher) with WU46 plasmid using established protocols. Surface expression of chimeric CEA_IL7 receptor WU46 is detected using Arcitumomab (Novus Biologicals, Cat. No. NBP2-52673) conjugated to Dylight 405 fluorophore (Novs Biologicals, Cat. No. 321-0010); concurrent expression of Thy1.1 is detected using an anti-Thy1.1 antibody conjugated to phycoerythrin (PE) (BioLegend, Cat. No. 202523) (FIG. 6).

Lentiviral Transduction of Target Cells

For initial validation, Jurkat (human T cell leukemia) cell lines cultured under standard tissue culture methods will be transduced with lentivirus with polybrene using standard practices.

Upon successful validation in Jurkat cells, lentivirus can also be used to transduce primary human T cells collected from donor leukopheresis products or therapeutic CAR-T cells using standard primary cell transduction protocols (Cooper, M. et al., Leukemia 2018).

The expression of our chimeric IL-7 receptors can be validated by flow cytometry using the same protocol highlighted in the section above.

Validation of Signaling

Jurkat cells, primary T cells, or CAR-T cells can be assessed for chimeric IL-7 signal transduction through the use of flow cytometry. T Lentivirus transduced cells will be stimulated with the appropriate cognate antigen for WU44, 45, or 46 (see Ligands section) using standard tissue culture protocols. The phosphorylation of Stat5 following stimulation is indicative of IL-7 receptor signaling. Chimeric IL-7 receptor activation can be visualized by flow cytometry using phospho-Stat5 (pStat5) intracellular staining and flow cytometry (Krutzik, P. O. et al, J Immunol, 2005; Hasegawa, D. et al, Blood Cancer J, 2013). Alternatively, phosphorylation of Stat5 can be visualized by western blotting.

Validation of Efficacy

Therapeutic CAR-T cells expressing CD19, or CD7 specific scFvs can be transduced with the chimeric IL-7 receptors or empty viral controls. Using murine models, mice are engrafted with leukemia tumor models followed by adoptive transfer of CAR-T cells. Tumor growth and mouse survival can be monitored using well established techniques (Cooper et al., Leukemia 2018).

The cognate ligands for chimeric IL-7 receptors can then be injected into these mice and changes to disease outcome assessed. It is expected that CAR-Ts expressing chimeric IL-7 receptors as disclosed herein and treated with stimulatory ligands will have increased longevity compared to untransduced controls, allowing for the continued protection of the host following a secondary challenge of leukemia (or lymphoma, or other cancer) engraftment. Such data would demonstrate that CAR-bearing immune effector cells comprising a chimeric IL-7, IL-2, or IL-15 receptor as disclosed herein would have advantages in the treatment of cancer in human and animal patients.

The validation methods described above can be modified for validation of chimeric IL-15 and IL-2 receptors.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A composition comprising a population of CAR-bearing immune effector cells, the cells comprising a transmembrane protein, the transmembrane protein comprising at least two recombinant chains, each recombinant chain comprising an endodomain, a transmembrane domain, and an ectodomain;
wherein the ectodomains of the at least two chains of the transmembrane protein interact together to bind to a drug or soluble protein;
wherein the endodomains of the at least two chains of the transmembrane protein together comprise a structure functionally similar to that of an IL-2 receptor endodomain, an IL-7 receptor endodomain, or an IL-15 receptor endodomain; and
wherein binding of the drug or soluble protein to the ectodomain activates IL-2, IL-7, or IL-15 signaling in CAR-bearing immune effector cells.

2. The composition of claim 1, wherein the ectodomains of the at least two chains of the transmembrane protein each comprise an antibody or binding portion thereof.

3. The composition of claim 1, wherein the binding portion of each antibody is chosen from a Fab, scFv, F(ab′)₂, minibody, or other binding arrangement of V_{H} and V_{L} chains or CDR-containing proteins.

4. The composition of claim 1, wherein the ectodomains of the at least two chains of the transmembrane protein each comprise a scFv.

5. The composition of any of claims 2-4, wherein the antibodies or binding portions thereof, or the scFvs, together comprise a drug- or soluble protein-recognition domain.

6. The composition of any of claims 1-5, wherein the soluble protein comprises two distinct epitopes.

7. The composition of claim 6, wherein the two distinct epitopes of the soluble protein bind to the drug- or soluble protein-recognition domain.

8. The composition of claim 7, wherein the binding of the transmembrane protein to the drug- or soluble protein-recognition domain initiates internal signaling.

9. The composition of any of claims 1-8, wherein the at least two chains of the transmembrane protein comprise:
a recombinant IL-7Rα chain or fragment thereof and a recombinant common γ chain or fragment thereof;
two recombinant IL-7Rα chains or fragments thereof;
a recombinant IL-2Rβ chain or fragment thereof and a recombinant common γ chain or fragment thereof; or
two recombinant IL-2Rβ chains or fragments thereof;
a recombinant IL-15Rβ chain or fragment thereof and a recombinant common γ chain or fragment thereof; or
two recombinant IL-15Rβ chains or fragments thereof.

10. The composition of claim 9, wherein the recombinant IL-7Rα chain or fragment thereof, IL-2Rβ chain or fragment thereof, or IL-15Rβ chain or fragment thereof comprises (i) a transmembrane domain, and (ii) an endodomain comprising an IL-7Rα, IL-2Rβ, or IL-15Rβ signaling domain.

11. The composition of any of claims 1-10, wherein the endodomains of the at least two chains of the transmembrane protein comprise:
a recombinant chain or fragment thereof comprising an IL-7Rα signaling domain and a recombinant chain or fragment thereof comprising a common γ signaling domain;
two recombinant chains or fragments thereof each comprising an IL-7Rα signaling domain;
a recombinant chain or fragment thereof comprising an IL-2Rβ signaling domain and a recombinant chain or fragment thereof comprising a common γ signaling domain;
two recombinant chains or fragments thereof each comprising an IL-2Rβ signaling domain;
a recombinant chain or fragment thereof comprising an IL-15Rβ signaling domain and a recombinant chain or fragment thereof comprising a common γ signaling domain; or
two recombinant chains or fragments thereof each comprising an IL-15Rβ signaling domain.

12. The composition of claim 11, wherein the recombinant common γ chain or fragment thereof comprises (i) a transmembrane domain, and (ii) an intracellular portion of a common γ chain.

13. The composition of any of claims 1-12, wherein dimerization of the recombinant chains initiates internal (IL-7R, IL-2R, or IL-15R) signaling.

14. The composition of claim 13, wherein either:
homodimerization of i) two recombinant chains or fragments thereof each comprising an IL-7Rα endodomain or signaling domain, ii) two recombinant chains or fragments thereof each comprising an IL-2Rβ endodomain or signaling domain, or iii) two recombinant chains or fragments thereof each comprising an IL-15Rβ endodomain or signaling domain; or
heterodimerization of iv) a recombinant chain or fragment thereof comprising an IL-7Rα endodomain or signaling domain and a recombinant chain or fragment thereof comprising a common γ endodomain or signaling domain, v) a recombinant chain or fragment thereof comprising an IL-2Rβ endodomain or signaling domain and a recombinant chain or fragment thereof comprising a common γ endodomain or signaling domain, and vi) a recombinant chain or fragment thereof comprising an IL-15Rβ endodomain or signaling domain and a recombinant chain or fragment thereof comprising a common γ endodomain or signaling domain
initiates internal (IL-7R, IL-2R, or IL-15R) signaling.

15. The composition of any of claims 1-14, wherein the drug or soluble protein comprises a recombinant human protein.

16. The composition of claim 15, wherein the recombinant human protein is modified to alter its function.

17. The composition of claim 16, wherein the altered function comprises modified stability, modified binding efficacy, modified natural function, modified specificity, modified immunogenicity, or modified half-life.

18. The composition of any of claims 1-17, wherein the drug or soluble protein is chosen from: an opioid antagonist, a vitamin, a cannabinoid, an antibiotic, dihydrostreptomycin, a coxib, a profen, fenclozic acid, fenclofenac, a NDRI antidepressant, a hydrazine MAOI, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs, a formin, an antihistamine, a 5HT4 agonist, Oxyphenisatine, nefazodone, levamisole (antihelminthic), Flosequinan (quinolone vasodilator), metamizole, dimethylamylamine (DMAA, Forthane), insulin (optionally inactivated), osteopontin or a form thereof, and a monoclonal antibody.

19. The composition of claim 18, wherein the drug or soluble protein is a monoclonal antibody, or a fragment thereof.

20. The composition of claim 19, wherein the drug or soluble protein is omalizumab.

21. A composition comprising a population of CAR-bearing immune effector cells, the cells comprising a transmembrane protein, the transmembrane protein comprising at least two protein chains, each chain comprising an endodomain, a transmembrane domain, and an ectodomain;
wherein the endodomains of the at least two chains of the transmembrane protein together comprise a structure functionally similar to that of an IL-2 receptor endodomain, an IL-7 receptor endodomain, or an IL-15 receptor endodomain; and
wherein administration of a chemical compound results in dimerization of the at least two protein chains and activates IL-2, IL-7, and/or IL-15 signaling in CAR-bearing immune effector cells.

22. The composition of claim 21 wherein the at least two protein chains comprise:
a recombinant IL-7Rα chain or fragment thereof and a recombinant common γ chain or fragment thereof;
two recombinant IL-7Rα chains or fragments thereof;
a recombinant IL-2Rβ chain or fragment thereof and a recombinant common γ chain or fragment thereof; or
two recombinant IL-2Rβ chains or fragments thereof;
a recombinant IL-15Rβ chain or fragment thereof and a recombinant common γ chain or fragment thereof; or
two recombinant IL-15Rβ chains or fragments thereof.

23. The composition of claim 21, wherein the recombinant IL-7Rα chain or fragment thereof, IL-2Rβ chain or fragment thereof, or IL-15Rβ chain or fragment thereof comprises (i) a transmembrane domain, and (ii) an endodomain comprising an IL-7Rα, IL-2Rβ, or IL-15Rβ signaling domain.

24. The composition of claim 21, wherein the endodomains of the at least two chains of the transmembrane protein comprise:
a recombinant chain or fragment thereof comprising an IL-7Rα signaling domain and a recombinant chain or fragment thereof comprising a common γ signaling domain;
two recombinant chains or fragments thereof each comprising an IL-7Rα signaling domain;
a recombinant chain or fragment thereof comprising an IL-2Rβ signaling domain and a recombinant chain or fragment thereof comprising a common γ signaling domain;
two recombinant chains or fragments thereof each comprising an IL-2Rβ signaling domain;
a recombinant chain or fragment thereof comprising an IL-15Rβ signaling domain and a recombinant chain or fragment thereof comprising a common γ signaling domain; or
two recombinant chains or fragments thereof each comprising an IL-15Rβ signaling domain.

25. The composition of any of claims 21-24, wherein:
the recombinant IL-7Rα chain(s) or fragment(s) thereof comprise(s) (i) a transmembrane domain, and (ii) a binding-protein binding domain and an endodomain or signaling domain of an IL-7Rα chain;
the recombinant IL-2Rβ chain(s) or fragment(s) thereof comprise(s) (i) a transmembrane domain, and (ii) a binding-protein binding domain and an endodomain or signaling domain of an IL-2Rβ chain; or
the recombinant IL-15Rβ chain(s) or fragment(s) thereof comprise(s) (i) a transmembrane domain, and (ii) a binding-protein binding domain and an endodomain or signaling domain of an IL-15Rβ chain.

26. The composition of any of claims 22-25, wherein the recombinant common γ chain or fragment thereof comprises (i) a transmembrane domain, and (ii) a binding-protein binding domain and an endodomain or signaling domain of a common γ chain.

27. The composition of any of claims 22-26, wherein the binding-protein binding domain is an FKBP binding domain.

28. The composition of claim 27, wherein the FKBP domain is located extracellularly, intracellularly between the signaling domain and the transmembrane domain, or at the terminus of the signaling domain distal to the plasma membrane.

29. The composition of any of claims 22-28, wherein (i) the recombinant IL-7Rα chain or fragment thereof, recombinant IL-2Rβ chain or fragment thereof, or recombinant IL-15Rβ chain or fragment thereof, or (ii) the common γ chain or fragment thereof, or (iii) both the recombinant IL-7Rα or IL-2Rβ or IL-15Rβ chain or fragment thereof and the common γ chain or fragment thereof further comprises an extracellular peptide tag.

30. The composition of claim 29, wherein the extracellular peptide tag comprises human truncated CD34.

31. The composition of any of claims 22-30, wherein the chemical compound comprises a dimerization agent.

32. The composition of claim 31, wherein the dimerization agent comprises FK1012.

33. The composition of any of claims 22-32, wherein dimerization of the at least two protein chains initiates internal (IL-7R, IL-2R, or IL-15R) signaling.

34. The composition of any of claims 242-33, wherein the ectodomain of at least one of the at least two chains of the transmembrane protein comprises an antibody or binding portion thereof.

35. The composition of claim 34, wherein the ectodomains of the at least two chains of the transmembrane protein each comprises an antibody or binding portion thereof.

36. The composition of claim 34, wherein the binding portion of the antibody is chosen from a Fab, scFv, F(ab′)₂, minibody, or other binding arrangement of V_{H} and V_{L} chains or CDR-containing proteins.

37. The composition of claim 35, wherein the binding portion of each antibody is chosen from a Fab, scFv, F(ab′)₂, minibody, or other binding arrangement of V_{H} and V_{L} chains or CDR-containing proteins.

38. The composition of claim 36, wherein the ectodomains of the at least one of the at least two chains of the transmembrane protein comprises a scFv.

39. The composition of claim 37, wherein the ectodomains of the at least two chains of the transmembrane protein each comprise a scFv.

40. The composition of any of claims 34-39, wherein the antibodie(s) or binding portion(s) thereof, or the scFv(s), individually or together comprise a drug- or soluble protein-recognition domain.

41. The composition of claim 40, wherein an epitope or epitopes of the soluble protein bind to the soluble protein-recognition domain.

42. The composition of claim 41, wherein the binding of the transmembrane protein to the drug- or soluble protein-recognition domain and dimerization of the at least two protein chains initiates internal (IL-7R, IL-2R, or IL-15R) signaling.

43. A composition comprising a population of CAR-bearing immune effector cells, the cells comprising a transmembrane protein, the transmembrane protein comprising at least two chains, each chain comprising an endodomain, a transmembrane domain, and an ectodomain;
wherein the ectodomains of the at least two chains of the transmembrane protein interact together to bind to a recombinant soluble protein comprising a first and a second domain;
wherein the first domain of the recombinant soluble protein binds to the ectodomain of one of the chains of the transmembrane protein, and the second domain of the recombinant soluble protein binds to the ectodomain of the other chain of the transmembrane protein;
wherein the endodomains of the at least two chains of the transmembrane protein together comprise a structure functionally similar to that of an IL-7 receptor endodomain or an IL-15 receptor endodomain; and
wherein binding of the recombinant soluble protein to the ectodomains activates IL-7 or IL-15 signaling in CAR-bearing immune effector cells.

44. The composition of claim 43, wherein the ectodomains of the at least two chains of the transmembrane protein each comprise an antibody or binding portion thereof.

45. The composition of claim 44, wherein the binding portion of each antibody is chosen from a Fab, scFv, F(ab′)₂, minibody, or other binding arrangement of V_{H} and V_{L} chains or CDR-containing proteins.

46. The composition of claim 45, wherein the ectodomains of the at least two chains of the transmembrane protein each comprise a scFv.

47. The composition of any of claims 43-46, wherein the antibodies or binding portions thereof, or the scFvs, together comprise a drug or recombinant protein recognition domain.

48. The composition of any of claims 43-47, wherein the recombinant soluble protein comprises two distinct epitopes.

49. The composition of claim 48, wherein the two distinct epitopes of the recombinant soluble protein bind to the recombinant protein recognition domain.

50. The composition of claim 49, wherein the binding of the transmembrane protein to the recombinant soluble protein recognition domain initiates internal (IL-7R or IL-15R) signaling.

51. The composition of claim 43-50 wherein the at least two protein chains comprise:
a recombinant IL-7Rα chain or fragment thereof and a recombinant common γ chain or fragment thereof;
two recombinant IL-7Rα chains or fragments thereof;
a recombinant IL-15Rβ chain or fragment thereof and a recombinant common γ chain or fragment thereof; or
two recombinant IL-15Rβ chains or fragments thereof.

52. The composition of any of claims 43-51, wherein:
the first domain of the recombinant soluble protein binds to the IL-7Rα chain or fragment thereof and the second domain of the recombinant protein binds to the common γ chain or fragment thereof;
the first domain of the recombinant soluble protein binds to one IL-7Rα chain or fragment thereof and the second domain of the recombinant protein binds to the other IL-7Rα chain or fragment thereof;
the first domain of the recombinant soluble protein binds to the IL-15Rβ chain or fragment thereof and the second domain of the recombinant soluble protein binds to the common γ chain or fragment thereof; or
the first domain of the recombinant soluble protein binds to one IL-15Rβ chain or fragment thereof and the second domain of the recombinant protein binds to the other IL-15Rβ chain or fragment thereof.

53. The composition of claim 52, wherein the recombinant IL-7Rα chain or fragment thereof or IL-15Rβ chain or fragment thereof comprises (i) a transmembrane domain, and (ii) an endodomain comprising an IL-7Rα or IL-15Rβ signaling domain.

54. The composition of claim any of claims 43-53, wherein the endodomains of the at least two chains of the transmembrane protein comprise:
a recombinant chain or fragment thereof comprising an IL-7Rα endodomain and a recombinant chain or fragment thereof comprising a common γ endodomain;
two recombinant chains or fragments thereof each comprising an IL-7Rα endodomain;
a recombinant chain or fragment thereof comprising an IL-15Rβ endodomain and a recombinant chain or fragment thereof comprising a common γ endodomain; or
two recombinant chains or fragments thereof each comprising an IL-15Rβ endodomain.

55. The composition of claim 54, wherein the recombinant common γ chain or fragment thereof comprises (i) a transmembrane domain, and (ii) an intracellular portion of a common γ chain.

56. The composition of any of claims 21-54, wherein the (recombinant) soluble protein comprises a recombinant human protein.

57. The composition of claim 56, wherein the recombinant human protein is modified to alter its function.

58. The composition of claim 57, wherein the altered function comprises modified stability, modified binding efficacy, modified natural function, modified specificity, modified immunogenicity, or modified half-life.

59. The composition of any of claims 21-58, wherein the drug or soluble protein is chosen from: an opioid antagonist, a vitamin, a cannabinoid, an antibiotic, dihydrostreptomycin, a coxib, a profen, fenclozic acid, fenclofenac, a NDRI antidepressant, a hydrazine MAOI, Benmoxin (Neuralex, Nerusil), Iproclozide (Sursum), Iproniazid (Marsilid), Isocarboxazid (Marplan), Mebanazine (Actomol), nialamide (Niamid), octamoxin (Ximaol, Nimaol), phenelzine (Nardil), Pheniprazine (Catron), Phenoxypropazine (Drazine), Pivhydrazine (Tersavid), Safrazine (Safra), sibutramine, phenylpropanolamine (decongestant, appetite suppressant), Pergolide (DRA), PPARs, a formin, an antihistamine, a 5HT4 agonist, Oxyphenisatine, nefazodone, levamisole (antihelminthic), Flosequinan (quinolone vasodilator), metamizole, dimethylamylamine (DMAA, Forthane), insulin (optionally inactivated), osteopontin or a form thereof, and a monoclonal antibody.

60. The composition of claim 59, wherein the drug or soluble protein is a monoclonal antibody, or a fragment thereof.

61. The composition of claim 60, wherein the drug or soluble protein is omalizumab.

62. The composition of any of claims 1-61, wherein the CAR-bearing immune effector cells are chosen from CAR-T cells, CAR-iNKT cells, CAR-NK cells, CAR-macrophage cells, iPSC derived CAR-T cells, and iPSC derived CAR-NK cells.

63. The composition of claim 62, wherein the CAR-bearing immune effector cells are CAR-T cells.

64. The composition of claim 62, wherein the CAR binds to (targets) an antigen chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

65. A method for treatment of cancer comprising:
a) administering a population of CAR-bearing immune effector cells to a patient in need thereof, wherein the CAR-bearing immune effector cells comprise a transmembrane protein of any of claims 1-40;
b) administering a drug or soluble protein capable of binding to the transmembrane protein;
wherein administration of the population of CAR-bearing immune effector cells and the drug or soluble enhances IL-2, IL-7, or IL-15 signaling in the patient.

66. The method as recited in claim 65, wherein the cancer is a hematologic malignancy.

67. The method as recited in claim 66, wherein the hematologic malignancy is a T-cell malignancy.

68. The method as recited in claim 67, wherein the T cell malignancy is T-cell acute lymphoblastic leukemia (T-ALL).

69. The method as recited in claim 67, wherein the T cell malignancy is non-Hodgkin's lymphoma.

70. The method as recited in claim 67, wherein the T cell malignancy is T-cell chronic lymphocytic leukemia (T-CLL).

71. The method as recited in claim 66, wherein the hematologic malignancy is a B-cell malignancy.

72. The method as recited in claim 71, wherein the B-cell malignancy is diffuse large B-cell lymphoma (DLBCL).

73. The method as recited in claim 66, wherein the hematologic malignancy is multiple myeloma.

74. The method as recited in claim 73, wherein the hematologic malignancy is acute myeloid leukemia (AML).

75. The method as recited in claim 65, wherein the cancer is a solid tumor.

76. A recombinant transmembrane protein, the transmembrane protein comprising at least two chains, each chain comprising an endodomain, a transmembrane domain, and an ectodomain;
wherein the ectodomains of the at least two chains of the transmembrane protein interact together to bind to a drug or soluble protein;
wherein the endodomains of the at least two chains of the transmembrane protein together comprise a structure functionally similar to that of an IL-2 receptor endodomain, an IL-7 receptor endodomain, or an IL-15 receptor endodomain; and
wherein binding of the drug or soluble protein to the ectodomain activates IL-2, IL-7, or IL-15 signaling in a cell.

77. The recombinant transmembrane protein of claim 76, wherein the ectodomains of the at least two chains of the transmembrane protein each comprise an antibody or binding portion thereof.

78. The recombinant transmembrane protein of claim 77, wherein the binding portion of each antibody is chosen from a Fab, scFv, F(ab′)₂, minibody, or other binding arrangement of V_{H} and V_{L} chains or CDR-containing proteins.

79. The recombinant transmembrane protein of claim 78, wherein the ectodomains of the at least two chains of the transmembrane protein each comprise a scFv.

80. The recombinant transmembrane protein of any of claims 77-79, wherein the antibodies or binding portions thereof, or the scFvs, together comprise a drug- or soluble protein-recognition domain.

81. A recombinant transmembrane protein, the protein comprising an endodomain, a transmembrane domain, and an ectodomain;
wherein the ectodomain of the receptor binds to a drug or soluble protein;
wherein the endodomain of the receptor comprises a structure functionally similar to that of an IL-7R, IL-2R, or IL-15R endodomain; and
wherein binding of the drug or soluble protein to the ectodomain is capable of activating IL-7, IL-2, or IL-15 signaling in a cell.

82. The transmembrane protein of any of claims 76-81, wherein the soluble protein comprises two distinct epitopes.

83. The transmembrane protein of claim 82, wherein the two distinct epitopes of the soluble protein bind to the drug or soluble protein recognition domain.

84. A recombinant nucleic acid encoding the transmembrane protein of any of claims 76-83.

85. A vector comprising the nucleic acid of claim 84.

86. The vector of claim 85 wherein the vector is a lentiviral plasmid vector.

87. A recombinant chimeric IL-7Rα, IL-15Rβ, IL-2Rβ, or IL-γc polypeptide chain, each comprising an endodomain, a transmembrane domain, and an ectodomain, wherein the extracellular domain is modified to comprise an scFv that recognizes and binds to an epitope of a drug or soluble protein that is not IL-7, IL-15, or IL-2.

88. The recombinant chimeric IL-7Rα, IL-15Rβ, IL-2Rβ, or IL-γc polypeptide of claim 87, wherein the transmembrane domain of the modified IL-7Rα polypeptide chain is hybridized to the extracellular domain using a CD8 linker domain.

89. A recombinant nucleic acid construct encoding the chimeric IL-7Rα, IL-15Rβ, IL-2Rβ, or IL-γc polypeptide chain of any of claims 87-88.

90. The recombinant nucleic acid construct of claim 89, wherein the coding sequence of the chimeric IL-7Rα, IL-15Rβ, IL-2Rβ, or IL-γc polypeptide chain is expressed in a lentiviral plasmid.

91. The recombinant nucleic acid construct of claim 89, wherein the construct comprises at least one element or domain selected from a Kozak sequence and a CD8a signal peptide; a human IgE CE3 extracellular domain; a CD8 linker extracellular domain; an IL-7Rα transmembrane domain; an IL-7Rα intracellular domain; a P2A-Thy1.1 co-expressed surface marker; an anti-4-Hydroxy-3-nitrophenyl (NP) 3B44 SCFV extracellular domain; and/or a CEA a2-b3 extracellular domain.

92. The recombinant nucleic acid construct of claim 89, wherein the construct comprises at least one element or domain selected from SEQ ID NOs:1-8.

93. The recombinant nucleic acid construct of claim 92, wherein the construct comprises:
SEQ ID NOs:1-6;
SEQ ID NOs:1 and 3-7; and/or
SEQ ID NOs:1, 3-6, and 8.

94. The recombinant nucleic acid construct of any of claims 90-93, wherein the lentiviral plasmid vector is pLVM-EF1a.

95. A host cell comprising the recombinant chimeric IL-7Rα, IL-15Rβ, IL-2Rβ, or IL-γc polypeptide of any of claims 87-88.

96. A host cell comprising the recombinant nucleic acid construct of any of claims 89-94.
